(19) United States
(12) Patent Application Publication
(10) Pub. No.: US 2026/0008719 A1
(43) Pub. Date: Jan. 8, 2026

(54) PHOSPHATE BASED GLASS FIBRES

(71) Applicant: UNIVERSITY OF SURREY, Guildford (GB)

(72) Inventors: Daniela CARTA, Guildford (GB); Agron Hoxha, Guildford (GB); Matthew Hardman, Guildford (GB); Farzad Foroutan, Guildford (GB)

(21) Appl. No.: 19/268,933

(22) Filed: Jul. 14, 2025

Related U.S. Application Data
(63) Continuation of application No. PCT/GB2024/050127, filed on Jan. 17, 2024.

(30) Foreign Application Priority Data
Jan. 18, 2023 (GB) 2300736.2

(51) Int. Cl. C03C 13/00; A61K 9/70; A61L 27/12; A61L 27/18; C03B 17/00; C03C 3/16; C03C 4/00

(52) U.S. Cl. CPC C03C 13/00; A61K 9/70; A61L 27/12; A61L 27/18; C03B 17/00; C03C 3/16; C03C 4/0014; A61L 2430/02; C03C 2213/02

(57) ABSTRACT

The invention relates to a fibre comprising or consisting of a porous phosphate-based glass (PBG). The PBG has an average pore diameter of less than 1,000 nm. The invention extends to methods of making the PBG, and medical uses thereof.

### CROSS REFERENCE TO RELATED APPLICATIONS

This application is a Continuation of the International Application No. PCT/GB2024/050127, filed Jan. 17, 2024, which claims priority to, and the benefit of, Great Britain Application No. GB 2300736.2, filed Jan. 18, 2023, the entire contents of each of which are incorporated by reference in their entirety for all purposes.

### FIELD

The present invention relates to phosphate-based glass, particularly porous phosphate-based glass. The invention extends to methods of producing the phosphate-based glass and medical uses thereof.

### BACKGROUND

Within the past few decades, a wide range of biomaterials have been developed for tissue engineering and drug delivery applications. The production of new materials that resemble the complex hierarchical structures of natural tissues to regenerate and repair of damaged or lost tissues is an extremely attractive field of research. Bioresorbable materials can be engineered as a temporary scaffold for cells to guide their proliferation and differentiation into the desired tissue. Drug molecules can also be delivered to their sites of action via a drug delivery carrier at such a concentration and rate that causes the minimum side effects and maximum therapeutic effects. There should be a proper loading of the drug into an appropriate drug delivery carrier and the carrier must be non-toxic with an adequate residence time in circulation to be able to reach the site of interest. The carriers should also be biodegradable, thereby enabling them to be easily cleared away by the body's physiological mechanisms, and thus avoids any chance of its accumulation within cells that may lead to cytotoxicity.

Recently, a wide range of bioresorbable polymers have been developed. However, just a limited number of them have been approved to be used for biomedical applications such as tissue engineering and drug delivery systems as the long-term effects of these polymeric structures are still under investigation. One of the main reasons for their limited applications can be related to the release of polymer fragments with heterogeneous chain-lengths during their degradation that might lead to toxicity. In addition, premature release of drug molecules before reaching a target site can be pharmacologically dangerous. Numerous studies have been performed on mesoporous silica-based materials that can provide broad range of advantages such as biocompatibility, high surface area, and high pore volume. The materials are classified as mesoporous due to their pore size, which range between 2 nm and 50 nm. The empty pores of these mesoporous materials can be simply filled by exposing them to a solution containing the drug molecules and allowing the molecules to diffuse therein. Mesoporous silica-based glass nanoparticles (MSGNs) have been emerging as a new interesting field for cancer therapy and have the ability to target cancerous cells to deliver the drug by functionalisation of their surface with specific ligands. However, the major disadvantages of MSGNs are attributed to the silanol groups interaction with the surface of the phospholipids of the red blood cell membranes resulting in haemolysis and metabolic changes that may lead to melanoma promotion. In addition, despite the verified biodegradability of silica, the issue of safe and complete exocytosis of MSGNs from the biological system has not been entirely addressed that cause major obstacles impeding their clinical translation.

Phosphate based glasses (PBGs) are a unique group of amorphous materials which are totally soluble in the human body fluid. For several years, PBGs containing Ca²⁺ and Na⁺ ions have been used as a passive host material for the controlled release of metal ions in veterinary treatments. As the ions released from the PBGs already exist in the body, very low toxicity and good biocompatibility are guaranteed. The degradation rate can be controlled from minutes to several months via changing their composition that offer the potential use of these glasses as a carrier in drug delivery application. Their high biocompatibility and controllable degradation rate can also offer a significant advantage over the current MSGNs drug delivery carriers.

Phosphate fibres are usually prepared via the traditional melt-spinning (MS) technique, where oxide powders are melted at temperatures >1100° C. followed by drawing fibres directly. However, MS is incompatible with thermally sensitive molecules, fails to deliver the high surface areas and porosity necessary for effective cell attachment and will reduce metal ions. Moreover, PBG fibres produced via MS are quite stiff making them unable to conform to a defect, such as a wound shape or bone defect.

### BRIEF DESCRIPTION OF THE FIGURES

For a better understanding of the present disclosure, and to show how embodiments of the same may be carried into effect, reference will now be made, by way of example, to the accompanying Figures.

FIG. 1A-1 shows a scanning electron microscopy (SEM) image of undoped coacervates glass powders calcined at 300° C., at 3500× magnification.

FIG. 1A-2 shows a scanning electron microscopy (SEM) image of undoped coacervates glass powders calcined at 300° C., at 15000× magnification.

FIG. 1B-1 shows a scanning electron microscopy (SEM) image of undoped coacervates fibres calcined at 300° C., at 3500× magnification.

FIG. 1B-2 shows a scanning electron microscopy (SEM) image of undoped coacervates fibres calcined at 300° C., at 25000× magnification.

FIG. 2A-1 shows a scanning electron microscopy (SEM) image of coacervates glass powders doped with 0.1% w/v F127, 0% EtOH, at 1200× magnification.

FIG. 2A-2 shows a scanning electron microscopy (SEM) image of coacervates glass powders doped with 0.1% w/v F127, 0% EtOH, as an enlarged view of a portion of FIG. 2A-1.

FIG. 2A-3 shows a scanning electron microscopy (SEM) image of coacervates glass powders doped with 0.1% w/v F127, 10% EOH, at 3500× magnification.

FIG. 2A-4 shows a scanning electron microscopy (SEM) image of coacervates glass powders doped with 0.1% w/v F127, 10% EOH, as an enlarged view of a portion of FIG. 2A-3.

FIG. 2A-5 shows a scanning electron microscopy (SEM) image of coacervates glass powders doped with 1% w/v F127, 0% EtOH, at 1200× magnification.

FIG. 2A-6 shows a scanning electron microscopy (SEM) image of coacervates glass powders doped with 1% w/v F127, 0% EtOH, as an enlarged view of a portion of FIG. 2A-5.

FIG. 2A-7 shows a scanning electron microscopy (SEM) image of coacervates glass powders doped with 1% w/v F127, 10% EtOH, at 3500× magnification.

FIG. 2A-8 shows a scanning electron microscopy (SEM) image of coacervates glass powders doped with 1% w/v F127, 10% EtOH, as an enlarged view of a portion of FIG. 2A-7.

FIG. 2A-9 shows a scanning electron microscopy (SEM) image of coacervates glass powders doped with 5% w/v F127, 20% EtOH, at 1200× magnification.

FIG. 2A-10 shows a scanning electron microscopy (SEM) image of coacervates glass powders doped with 5% w/v F127, 20% EtOH, as an enlarged view of a portion of FIG. 2A-9.

FIG. 2A-11 shows a scanning electron microscopy (SEM) image of coacervates glass powders doped with 5% w/v F127, 70% EtOH, at 6500× magnification.

FIG. 2A-12 shows a scanning electron microscopy (SEM) image of coacervates glass powders doped with 5% w/v F127, 70% EtOH, as an enlarged view of a portion of FIG. 2A-11.

FIG. 2A-13 shows a scanning electron microscopy (SEM) image of coacervates glass powders doped with 0.1% w/v F127, 20% EtOH, 3500× magnification.

FIG. 2A-14 shows a scanning electron microscopy (SEM) image of coacervates glass powders doped with 0.1% w/v F127, 20% EtOH, as an enlarged view of a portion of FIG. 2A-13.

FIG. 2A-15 shows a scanning electron microscopy (SEM) image of coacervates glass powders doped with 0.1% w/v F127, 70% EtOH, 3500× magnification.

FIG. 2A-16 shows a scanning electron microscopy (SEM) image of coacervates glass powders doped with 0.1% w/v F127, 70% EtOH, as an enlarged view of a portion of FIG. 2A-15.

FIG. 2A-17 shows a scanning electron microscopy (SEM) image of coacervates glass powders doped with 1% w/v F127, 20% EtOH, 15000× magnification.

FIG. 2A-18 shows a scanning electron microscopy (SEM) image of coacervates glass powders doped with 1% w/v F127, 20% EtOH, as an enlarged view of a portion of FIG. 2A-17.

FIG. 2A-19 shows a scanning electron microscopy (SEM) image of coacervates glass powders doped with 1% w/v F127, 70% EtOH, 6500× magnification.

FIG. 2A-20 shows a scanning electron microscopy (SEM) image of coacervates glass powders doped with 5% w/v F127, 20% EtOH, 3500× magnification.

FIG. 2A-21 shows a scanning electron microscopy (SEM) image of coacervates glass powders doped with 5% w/v F127, 20% EtOH, as an enlarged view of a portion of FIG. 2A-20.

FIG. 2A-22 shows a scanning electron microscopy (SEM) image of coacervates glass powders doped with 5% w/v F127, 70% EtOH, 15000× magnification.

FIG. 2B-1 shows a scanning electron microscopy (SEM) image of coacervates glass fibres doped with 0.1% w/v F127, 0% EtOH, 2000× magnification.

FIG. 2B-2 shows a scanning electron microscopy (SEM) image of coacervates glass fibres doped with 0.1% w/v F127, 0% EtOH, as an enlarged view of a portion of FIG. 2B-1.

FIG. 2B-3 shows a scanning electron microscopy (SEM) image of coacervates glass fibres doped with 1% w/v F127, 0% EtOH, 1200× magnification.

FIG. 2B-4 shows a scanning electron microscopy (SEM) image of coacervates glass fibres doped with 1% w/v F127, 0% EtOH, as an enlarged view of a portion of FIG. 2B-3.

FIG. 2B-5 shows a scanning electron microscopy (SEM) image of coacervates glass fibres doped with 5% w/v F127, 0% EtOH, 1200× magnification.

FIG. 2B-6 shows a scanning electron microscopy (SEM) image of coacervates glass fibres doped with 5% w/v F127, 0% EtOH, as an enlarged view of a portion of FIG. 2B-5.

FIG. 3A-1 shows the diameter of the pores in the coacervates glass powders produced using 10% (v/v) ethanol doped with 0.1% w/v F127.

FIG. 3A-2 shows the diameter of the pores in the coacervates glass powders produced using 10% (v/v) ethanol doped with 1% w/v F127.

FIG. 3A-3 shows the diameter of the pores in the coacervates glass powders produced using 10% (v/v) ethanol doped with 5% w/v F127.

FIG. 3B-1 shows the diameter of the pores in the coacervates glass powders produced using 20% (v/v) ethanol doped with 0.1% w/v F127.

FIG. 3B-2 shows the diameter of the pores in the coacervates glass powders produced using 20% (v/v) ethanol doped with 1% w/v F127.

FIG. 3B-3 shows the diameter of the pores in the coacervates glass powders produced using 20% (v/v) ethanol doped with 5% w/v F127.

FIG. 3C-1 shows the diameter of the pores in the coacervates glass powders produced using 70% (v/v) ethanol doped with 0.1% w/v F127.

FIG. 3C-2 shows the diameter of the pores in the coacervates glass powders produced using 70% (v/v) ethanol doped with 1% w/v F127.

FIG. 3C-3 shows the diameter of the pores in the coacervates glass powders produced using 70% (v/v) ethanol doped with 5% w/v F127.

FIG. 3D-1 shows the diameter of the pores in the coacervates glass fibres produced using 0% (v/v) ethanol, doped with 0.1% w/v F127.

FIG. 3D-2 shows the diameter of the pores in the coacervates glass fibres produced using 0% (v/v) ethanol, doped with 1% w/v F127.

FIG. 3D-3 shows the diameter of the pores in the coacervates glass fibres produced using 0% (v/v) ethanol, doped with 5% w/v F127.

FIG. 4-1 shows a scanning electron microscopy (SEM) image of 0.1% w/v F127 (10% ethanol) coacervate powder showing a porous microsphere, 800× magnification.

FIG. 4-2 shows a scanning electron microscopy (SEM) image of 0.1% w/v F127 (10% ethanol) coacervate powder showing a porous microsphere, 20000× magnification.

FIG. 5A-1 shows a scanning electron microscopy (SEM) image of coacervates glass powders doped with 0.1% w/v P123, 0% EtOH, 1200× magnification.

FIG. 5A-2 shows a scanning electron microscopy (SEM) image of coacervates glass powders doped with 0.1% w/v P123, 0% EtOH, as an enlarged view of a portion of FIG. 5A-1.

FIG. 5A-3 shows a scanning electron microscopy (SEM) image of coacervates glass powders doped with 0.1% w/v P123, 20% EtOH, 1200× magnification.

FIG. 5A-4 shows a scanning electron microscopy (SEM) image of coacervates glass powders doped with 0.1% w/v P123, 20% EtOH, as an enlarged view of a portion of FIG. 5A-3.

FIG. 5A-5 shows a scanning electron microscopy (SEM) image of coacervates glass powders doped with 1% w/v P123, 0% EtOH, 1500× magnification.

FIG. 5A-6 shows a scanning electron microscopy (SEM) image of coacervates glass powders doped with 1% w/v P123, 0% EtOH, as an enlarged view of a portion of FIG. 5A-5.

FIG. 5A-7 shows a scanning electron microscopy (SEM) image of coacervates glass powders doped with 1% w/v P123, 20% EtOH, 1500× magnification.

FIG. 5A-8 shows a scanning electron microscopy (SEM) image of coacervates glass powders doped with 1% w/v P123, 20% EtOH, as an enlarged view of a portion of FIG. 5A-7.

FIG. 5A-9 shows a scanning electron microscopy (SEM) image of coacervates glass powders doped with 5% w/v P123, 0% EtOH, 1000× magnification.

FIG. 5A-10 shows a scanning electron microscopy (SEM) image of coacervates glass powders doped with 5% w/v P123, 0% EtOH, as an enlarged view of a portion of FIG. 5A-9.

FIG. 5A-11 shows a scanning electron microscopy (SEM) image of coacervates glass powders doped with 5% w/v P123, 20% EtOH, 1200× magnification.

FIG. 5A-12 shows a scanning electron microscopy (SEM) image of coacervates glass powders doped with 5% w/v P123, 20% EtOH, as an enlarged view of a portion of FIG. 5A-11.

FIG. 5B-1 shows a scanning electron microscopy (SEM) image of coacervates glass fibres doped with 0.1% w/v P123, 0% EtOH, 2500× magnification.

FIG. 5B-2 shows a scanning electron microscopy (SEM) image of coacervates glass fibres doped with 0.1% w/v P123, 0% EtOH, as an enlarged view of a portion of FIG. 5B-1.

FIG. 5B-3 shows a scanning electron microscopy (SEM) image of coacervates glass fibres doped with 1% w/v P123, 0% EtOH, 5000× magnification.

FIG. 5B-4 shows a scanning electron microscopy (SEM) image of coacervates glass fibres doped with 1% w/v P123, 0% EtOH, as an enlarged view of a portion of FIG. 5B-3.

FIG. 5B-5 shows a scanning electron microscopy (SEM) image of coacervates glass fibres doped with 5% w/v P123, 0% EtOH, 2500× magnification.

FIG. 5B-6 shows a scanning electron microscopy (SEM) image of coacervates glass fibres doped with 5% w/v P123, 0% EtOH, as an enlarged view of a portion of FIG. 5B-5.

FIG. 6-1 shows a scanning electron microscopy (SEM) image of 5% w/v F127 (0% ethanol) coacervate fibres showing aligned nanopores, 2500× magnification, with aligned nanopores 601.

FIG. 6-2 shows a scanning electron microscopy (SEM) image of 5% w/v F127 (0% ethanol) coacervate fibres showing aligned nanopores, as an enlarged view of a portion of FIG. 6-1, with aligned nanopores 601.

FIG. 7A-1 shows the diameter of the pores in the coacervates glass powders produced using 20% (v/v) ethanol doped with 0.1% w/v P123.

FIG. 7A-2 shows the diameter of the pores in the coacervates glass powders produced using 20% (v/v) ethanol doped with 1% w/v P123.

FIG. 7A-3 shows the diameter of the pores in the coacervates glass powders produced using 20% (v/v) ethanol doped with 5% w/v P123.

FIG. 7B-1 shows the diameter of the pores in the coacervates glass fibres produced using 0% (v/v) ethanol (B), doped with 1% w/v P123.

FIG. 7B-2 shows the diameter of the pores in the coacervates glass fibres produced using 0% (v/v) ethanol (B), doped with 5% w/v P123.

FIG. 8-1 shows a scanning electron microscopy (SEM) image of coacervates glass powders (20% ethanol) calcinated at 300° C. with a ramp speed of 0.5° C./min and doped with 0.1% w/v F127, 1200× magnification.

FIG. 8-2 shows a scanning electron microscopy (SEM) image of coacervates glass powders (20% ethanol) calcinated at 300° C. with a ramp speed of 0.5° C./min and doped with 0.1% w/v F127, as an enlarged view of a portion of FIG. 8-1.

FIG. 8-3 shows a scanning electron microscopy (SEM) image of coacervates glass powders (20% ethanol) calcinated at 300° C. with a ramp speed of 5° C./min and doped with 0.1% w/v F127, 1200× magnification.

FIG. 8-4 shows a scanning electron microscopy (SEM) image of coacervates glass powders (20% ethanol) calcinated at 300° C. with a ramp speed of 5° C./min and doped with 0.1% w/v F127, as an enlarged view of a portion of FIG. 8-3.

FIG. 8-5 shows a scanning electron microscopy (SEM) image of coacervates glass powders (20% ethanol) calcinated at 300° C. with a ramp speed of 0.5° C./min and doped with 1% w/v F127, 1200× magnification.

FIG. 8-6 shows a scanning electron microscopy (SEM) image of coacervates glass powders (20% ethanol) calcinated at 300° C. with a ramp speed of 0.5° C./min and doped with 1% w/v F127, as an enlarged view of a portion of FIG. 8-5.

FIG. 8-7 shows a scanning electron microscopy (SEM) image of coacervates glass powders (20% ethanol) calcinated at 300° C. with a ramp speed of 5° C./min and doped with 1% w/v F127, 1200× magnification.

FIG. 8-8 shows a scanning electron microscopy (SEM) image of coacervates glass powders (20% ethanol) calcinated at 300° C. with a ramp speed of 5° C./min and doped with 1% w/v F127, as an enlarged view of a portion of FIG. 8-7.

FIG. 8-9 shows a scanning electron microscopy (SEM) image of coacervates glass powders (20% ethanol) calcinated at 300° C. with a ramp speed of 0.5° C./min and doped with 5% w/v F127, 1200× magnification.

FIG. 8-10 shows a scanning electron microscopy (SEM) image of coacervates glass powders (20% ethanol) calcinated at 300° C. with a ramp speed of 0.5° C./min and doped with 5% w/v F127, as an enlarged view of a portion of FIG. 8-9.

FIG. 8-11 shows a scanning electron microscopy (SEM) image of coacervates glass powders (20% ethanol) calcinated at 300° C. with a ramp speed of 5° C./min and doped with 5% w/v F127, 1200× magnification.

FIG. 8-12 shows a scanning electron microscopy (SEM) image of coacervates glass powders (20% ethanol) calcinated at 300° C. with a ramp speed of 5° C./min and doped with 5% w/v F127, as an enlarged view of a portion of FIG. 8-11.

FIG. 9-1 shows a scanning electron microscopy (SEM) image of MgCa—Ag coacervates glass powders (5% ethanol) containing 1% w/v P123, undoped, 1200× magnification.

FIG. 9-2 shows a scanning electron microscopy (SEM) image of MgCa—Ag coacervates glass powders (5% ethanol) containing 1% w/v P123, undoped, as an enlarged view of a portion of FIG. 9-1.

FIG. 9-3 shows a scanning electron microscopy (SEM) image of MgCa—Ag coacervates glass powders (5% ethanol) containing 1% w/v P123, undoped.

FIG. 9-4 shows a scanning electron microscopy (SEM) image of MgCa—Ag coacervates glass powders (5% ethanol) containing 1% w/v P123, 1% Ag, 1200× magnification.

FIG. 9-5 shows a scanning electron microscopy (SEM) image of MgCa—Ag coacervates glass powders (5% ethanol) containing 1% w/v P123, 1% Ag, as an enlarged view of a portion of FIG. 9-4.

FIG. 9-6 shows a scanning electron microscopy (SEM) image of MgCa—Ag coacervates glass powders (5% ethanol) containing 1% w/v P123, 1% Ag.

FIG. 9-7 shows a scanning electron microscopy (SEM) image of MgCa—Ag coacervates glass powders (5% ethanol) containing 1% w/v P123, 3% Ag, 1200× magnification.

FIG. 9-8 shows a scanning electron microscopy (SEM) image of MgCa—Ag coacervates glass powders (5% ethanol) containing 1% w/v P123, 3% Ag, as an enlarged view of a portion of FIG. 9-7.

FIG. 9-9 shows a scanning electron microscopy (SEM) image of MgCa—Ag coacervates glass powders (5% ethanol) containing 1% w/v P123, 3% Ag.

FIG. 9-10 shows a scanning electron microscopy (SEM) image of MgCa—Ag coacervates glass powders (5% ethanol) containing 1% w/v P123, 5% Ag, 1200× magnification.

FIG. 9-11 shows a scanning electron microscopy (SEM) image of MgCa—Ag coacervates glass powders (5% ethanol) containing 1% w/v P123, 5% Ag, as an enlarged view of a portion of FIG. 9-10.

FIG. 9-12 shows a scanning electron microscopy (SEM) image of MgCa—Ag coacervates glass powders (5% ethanol) containing 1% w/v P123, 5% Ag.

FIG. 10-1 shows the results of X-ray diffraction (XRD) analysis for MgCa—Ag coacervates glass fibres (from top to bottom: 5% Ag, 0% EtOH; 3% Ag, 0% EtOH; 1% Ag, 0% EtOH; undoped 0% EtOH; 5% Ag, 5% EtOH; 3% Ag, 5% EtOH; 1% Ag, 5% EtOH; undoped 5% EtOH).

FIG. 10-2 shows the results of X-ray diffraction (XRD) analysis for MgCa—Ag coacervates glass powders (from top to bottom: 5% Ag, 0% EtOH; 3% Ag, 0% EtOH; 1% Ag, 0% EtOH; undoped 0% EtOH; 5% Ag, 5% EtOH; 3% Ag, 5% EtOH; 1% Ag, 5% EtOH; undoped 5% EtOH).

FIG. 11A shows the release of calcium from MgCa—Ag coacervates glass fibres (5% ethanol) containing 1% w/v P123.

FIG. 11B shows the release of sodium from MgCa—Ag coacervates glass fibres (5% ethanol) containing 1% w/v P123.

FIG. 11C shows the release of magnesium from MgCa—Ag coacervates glass fibres (5% ethanol) containing 1% w/v P123.

FIG. 11D shows the release of silver from MgCa—Ag coacervates glass fibres (5% ethanol) containing 1% w/v P123.

FIG. 12 representation of the ex vivo % wound closure of 2 mm excisional wounds on human skin biopsies exposed to solutions comprising either 1% (w/v) or 2.5% (w/v) MgCa—Ag fibre (F) or powder (P) (5% (v/v) ethanol, containing 1% w/v P123) which were either undoped or doped with 1, 3 or 5 mol % Ag. *=P<0.05, **=P<0.01, ***=P<0.001.

FIG. 13 shows confocal images of human skin biopsies demonstrating the wound closure of 2 mm excisional wounds as a result of the exposure to MgCa—Ag fibre and powder solutions (top left: no treatment; top right: 0% Ag MgCa fibre (1%); bottom left: 1% Ag MgCa fibre (1%); bottom right: 1% Ag MgCa powder (1%)).

FIG. 14A representation of the ex vivo % wound closure of 2 mm excisional wounds on human skin biopsies exposed to solutions comprising MgCa—Ag or Ca—Ag powder (5% (v/v) ethanol, containing 1% w/v P123) which were either undoped or doped with 1, 3 or 5 mol % Ag, the solutions comprised 0.1 wt % fibres or powder and were diluted in growth media at a volumetric ratio of 1:100.

FIG. 14B representation of the ex vivo % wound closure of 2 mm excisional wounds on human skin biopsies exposed to solutions comprising MgCa—Ag or Ca—Ag fibres (5% (v/v) ethanol, containing 1% w/v P123) which were either undoped or doped with 1, 3 or 5 mol % Ag, the solutions comprised 0.1 wt % fibres or powder and were diluted in growth media at a volumetric ratio of 1:100.

FIG. 15A shows the ability of the MgCa—Ag fibre (f) and powder (p) solutions to inhibit S. aureus. The fibres and powders had been prepared using 5% (v/v) ethanol and 1% w/v P123. Solutions of the glass fibres or powder (0.1 wt %) were diluted in growth media at a volumetric ratio of 1:100.

FIG. 15B shows the ability of the MgCa—Ag fibre (f) and powder (p) solutions to inhibit S. aureus. The fibres and powders had been prepared using 5% (v/v) ethanol and 1% w/v P123. Solutions of the glass fibres or powder (0.1 wt %) were diluted in growth media at a volumetric ratio of 3:100.

FIG. 15C shows the ability of the MgCa—Ag fibre (f) and powder (p) solutions to inhibit S. aureus. The fibres and powders had been prepared using 5% (v/v) ethanol and 1% w/v P123. Solutions of the glass fibres or powder (0.1 wt %) were diluted in growth media at a volumetric ratio of 5:100.

FIG. 16A shows the ability of the MgCa—Ag fibre (f) and powder (p) solutions to inhibit E. coli. The fibres and powders had been prepared using 5% (v/v) ethanol and 1% w/v P123. The solutions were provided at a weight ratio of glass fibres or powder to growth media of 1:100.

FIG. 16B shows the ability of the MgCa—Ag fibre (f) and powder (p) solutions to inhibit E. coli. The fibres and powders had been prepared using 5% (v/v) ethanol and 1% w/v P123. The solutions were provided at a weight ratio of glass fibres or powder to growth media of 3:100.

FIG. 16C shows the ability of the MgCa—Ag fibre (f) and powder (p) solutions to inhibit E. coli. The fibres and powders had been prepared using 5% (v/v) ethanol and 1% w/v P123. The solutions were provided at a weight ratio of glass fibres or powder to growth media of 5:100.

FIG. 17A shows the results of a cytotoxicity study for the MgCa—Ag and Ca—Ag powder (P) solutions. The MgCa—Ag powders were prepared using 5% (v/v) ethanol and 1% w/v P123. The solutions were provided at a weight ratio of glass powder to growth media of 1:100.

FIG. 17B shows the results of a cytotoxicity study for the MgCa—Ag and Ca—Ag fibre (F) solutions. The MgCa—Ag fibres had been prepared using 5% (v/v) ethanol and 1% w/v P123. The solutions were provided at a weight ratio of glass fibres to growth media of 1:100.

FIG. 17C shows the results of a cytotoxicity study for the MgCa—Ag and Ca—Ag fibre (F) and powder (P) solutions. The MgCa—Ag fibres and powders had been prepared using 5% (v/v) ethanol and 1% w/v P123. The solutions were provided at a weight ratio of glass fibres or powder to growth media of 3:100.

FIG. 17D shows the results of a cytotoxicity study for the MgCa—Ag and Ca—Ag fibre (F) and powder (P) solutions. The MgCa—Ag fibres and powders had been prepared using 5% (v/v) ethanol and 1% w/v P123. The solutions were provided at a weight ratio of glass fibres or powder to growth media of 5:100.

FIG. 18 shows the ability of porous and non-porous MgCa—Ag 5% fibre (F) and powder (P) solutions and non-porous Ca—Ag 5% fibre (F) and powder (P) solutions to inhibit E. coli. The solutions were provided at a weight ratio of glass fibres or powder to growth media of 1:100.

FIG. 19 shows the results of a cytotoxicity study of porous and non-porous MgCa—Ag 5% fibre (F) and powder (P) solutions and non-porous Ca—Ag 5% fibre (F) and powder (P) solutions. The solutions were provided at a weight ratio of glass fibres or powder to growth media of 1:100.

FIG. 20A shows XRD Patterns of COA-Ga-123-P uncal (top to bottom: COA-10% Ga-123-P uncal, COA-5% Ga-123-P uncal, COA-3% Ga-123-P uncal, COA-1% Ga-123-P uncal, COA-0.5% Ga-123-P uncal, COA-0.2% Ga-123-P uncal, COA-0% Ga-123-P uncal).

FIG. 20B shows XRD Patterns of COA-Ga-123-P cal (top to bottom: COA-10% Ga-123-P cal, COA-5% Ga-123-P cal, COA-3% Ga-123-P cal, COA-1% Ga-123-P cal, COA-0.5% Ga-123-P cal, COA-0.2% Ga-123-P cal, COA-0% Ga-123-P cal).

FIG. 20C shows XRD Patterns of COA-Ga-123-F uncal (top to bottom: COA-1% Ga-123-F, COA-0.5% Ga-123-F, COA-0.2% Ga-123-F, COA-0% Ga-123-F).

FIG. 20D shows XRD Patterns of COA-Ga-123-F cal (top to bottom: COA-1% Ga-123-F cal, COA-0.5% Ga-123-F cal, COA-0.2% Ga-123-F cal, COA-0% Ga-123-F cal).

FIG. 21A shows FT-IR spectra of COA-Ga-123-p-uncal (top to bottom: COA-10% Ga-123-p-uncal, COA-5% Ga-123-p-uncal, COA-3% Ga-123-p-uncal, COA-1% Ga-123-p-uncal, COA-0.5% Ga-123-p-uncal, COA-0.2% Ga-123-p-uncal, COA-0% Ga-123-p-uncal).

FIG. 21B shows FT-IR spectra of COA-Ga-123-p-cal (top to bottom: COA-5% Ga-123-p-cal, COA-3% Ga-123-p-cal, COA-1% Ga-123-p-cal, COA-0.5% Ga-123-p-cal, COA-0.2% Ga-123-p-cal, COA-0% Ga-123-p-cal).

FIG. 21C shows FT-IR spectra of COA-Ga-123-F uncal (top to bottom: COA-1% Ga-123-F, COA-0.5% Ga-123-F, COA-0.2% Ga-123-F, COA-0% Ga-123-F).

FIG. 21D shows FT-IR spectra of COA-Ga-123-F cal (top to bottom: COA-1% Ga-123-F cal, COA-0.5% Ga-123-F cal, COA-0.2% Ga-123-F cal, COA-0% Ga-123-F cal).

FIG. 22A shows a scanning electron microscopy (SEM) image of COA-0% Ga-123-P uncal.

FIG. 22B shows a scanning electron microscopy (SEM) image of COA-0% Ga-123-P cal.

FIG. 22C shows a scanning electron microscopy (SEM) image of COA-0.2% Ga-123-P cal.

FIG. 22D shows a scanning electron microscopy (SEM) image of COA-0.5% Ga-123-Pcal.

FIG. 22E shows a scanning electron microscopy (SEM) image of COA-1% Ga-123-P cal.

FIG. 22F shows a scanning electron microscopy (SEM) image of COA-3% Ga-123-P cal.

FIG. 23A shows a scanning electron microscopy (SEM) image of COA-0% Ga-123-F uncal.

FIG. 23B shows a scanning electron microscopy (SEM) image of COA-0.2% Ga-123-F uncal.

FIG. 23C shows a scanning electron microscopy (SEM) image of COA-0.5% Ga-123-F uncal.

FIG. 23D shows a scanning electron microscopy (SEM) image of COA-C0% Ga-123-F cal.

FIG. 23E shows a scanning electron microscopy (SEM) image of COA-C0% Ga-123-F cal.

FIG. 23F shows a scanning electron microscopy (SEM) image of COA-0.5% Ga-123-F cal.

FIG. 23G shows a scanning electron microscopy (SEM) image of COA-0.5% Ga-123-F cal.

FIG. 23H shows a scanning electron microscopy (SEM) image of COA-1% Ga-123-F cal.

FIG. 24A shows Raman spectra of COA-Ga-123-P uncal (top to bottom: COA-10% Ga-123-P, COA-5% Ga-123-P, COA-3% Ga-123-P, COA-1% Ga-123-P, COA-0.5% Ga-123-P, COA-0.2% Ga-123-P, COA-0Ga-123-P).

FIG. 24B shows Raman spectra of COA-Ga-123-P cal (top to bottom: COA-5% Ga-123-P cal, COA-3% Ga-123-P cal, COA-1% Ga-123-P cal, COA-0.5% Ga-123-P cal, COA-0.2% Ga-123-P cal, COA-0% Ga-123-P cal).

FIG. 24C shows Raman spectra of COA-Ga-123-F uncal (top to bottom: COA-1% Ga-123-F uncal, COA-0.5% Ga-123-F uncal, COA-0.2% Ga-123-F uncal, COA-0% Ga-123-F uncal).

FIG. 24D shows Raman spectra of COA-Ga-123-F cal (top to bottom: COA-1% Ga-123-F cal, COA-0.5% Ga-123-F cal, COA-0.2% Ga-123-F cal, COA-0% Ga-123-F cal).

FIG. 25A shows a scanning electron microscopy (SEM) image of COA-0% Ga-Calcined.

FIG. 25B shows a scanning electron microscopy (SEM) image of COA-0.2% Ga—P calcined.

FIG. 25C shows a scanning electron microscopy (SEM) image of COA-0.5% Ga-calcined.

FIG. 25D shows a scanning electron microscopy (SEM) image of COA-1% Ga—P calcined.

FIG. 26A shows a scanning electron microscopy (SEM) image COA-123-P uncalcined.

FIG. 26B shows a scanning electron microscopy (SEM) image COA-123-P cal.

FIG. 26C shows a scanning electron microscopy (SEM) image COA-0.2% Ce-123-P cal.

FIG. 26D shows a scanning electron microscopy (SEM) image COA-0.5% Ce-123-P cal.

FIG. 26E shows a scanning electron microscopy (SEM) image COA-1% Ce-123-P cal.

FIG. 26F shows a scanning electron microscopy (SEM) image COA-3% Ce-123-P cal.

FIG. 27A shows a scanning electron microscopy (SEM) image of COA-0% Ce-123-f uncal.

FIG. 27B shows a scanning electron microscopy (SEM) image of COA-0.2% Ce-123-f uncal.

FIG. 27C shows a scanning electron microscopy (SEM) image of COA-0.5% Ce123-F uncal.

FIG. 27D shows a scanning electron microscopy (SEM) image of 1% Ce-123-F uncal.

FIG. 27E shows a scanning electron microscopy (SEM) image of COA-0% Ce-123-F cal.

FIG. 27F shows a scanning electron microscopy (SEM) image of COA-0.2% Ce-123-F cal.

FIG. 27G shows a scanning electron microscopy (SEM) image COA-0.5% Ce-123-F cal.

FIG. 27H shows a scanning electron microscopy (SEM) image COA-1% Ce-123-F cal.

FIG. 28A shows ion release in DW of Na from PBG. Error bars indicate the mean±standard deviation (n=3).

FIG. 28B shows ion release in DW of Na from PBGF. Error bars indicate the mean±standard deviation (n=3).

FIG. 28C shows ion release in DW of Ca from PBG. Error bars indicate the mean±standard deviation (n=3).

FIG. 28D shows ion release in DW of Ca from PBGF. Error bars indicate the mean±standard deviation (n=3).

FIG. 28E shows ion release in DW of P from PBG. Error bars indicate the mean±standard deviation (n=3).

FIG. 28F shows ion release in DW of P from PBGF. Error bars indicate the mean±standard deviation (n=3).

FIG. 28G shows ion release in DW of Ga from PBG. Error bars indicate the mean±standard deviation (n=3).

FIG. 28H shows ion release in DW of Ga from PBGF. Error bars indicate the mean±standard deviation (n=3).

FIG. 29A shows ion release in DW of Na from PBG. Error bars indicate the mean±standard deviation (n=3).

FIG. 29B shows ion release in DW of Na from PBGF. Error bars indicate the mean±standard deviation (n=3).

FIG. 29C shows ion release in DW of Ca from PBG. Error bars indicate the mean±standard deviation (n=3).

FIG. 29D shows ion release in DW of Ca from PBGF. Error bars indicate the mean±standard deviation (n=3).

FIG. 29E shows ion release in DW of P from PBG. Error bars indicate the mean±standard deviation (n=3).

FIG. 29F shows ion release in DW of P from PBGF. Error bars indicate the mean±standard deviation (n=3).

FIG. 29G shows ion release in DW of Ce from PBG. Error bars indicate the mean±standard deviation (n=3).

FIG. 29H shows ion release in DW of Ce from PBGF. Error bars indicate the mean±standard deviation (n=3).

FIG. 30A shows antibacterial activity of COA-Ga-123 PBGFs against E. coli expressed as the mean±standard deviation (error bars) (*=p<0.05, ****=p<0.0001).

FIG. 30B shows antibacterial activity of COA-Ga-123 PBGs against E. coli expressed as the mean±standard deviation (error bars) (*=p<0.05, ****=p<0.0001).

FIG. 30C shows antibacterial activity of COA-Ga-123 PBGFs against S. aureus expressed as the mean±standard deviation (error bars) (*=p<0.05, ****=p<0.0001).

FIG. 30D shows antibacterial activity of COA-Ga-123 PBGs against S. aureus expressed as the mean±standard deviation (error bars) (*=p<0.05, ****=p<0.0001).

FIG. 31 shows viability of HACATS cells in Ga-doped-PBG (“P”) and PBGF (“F”) over 24 h.

FIG. 32A shows the results of scratch test for porous Ga-doped PBG (0, 0.2%, 0.5% and 1% Ga) after 24 h.

FIG. 32B shows the results of scratch test for porous Ga-doped PBGF (0, 0.2%, 0.5% and 1% Ga) after 24 h.

FIG. 33A shows an image of an in vitro assay using 0% Ga-123-P cal, depicting wound closure after 24 h.

FIG. 33B shows an image of an in vitro assay using 0.2% Ga-123-P cal, depicting wound closure after 24 h.

FIG. 33C shows an image of an in vitro assay using 0.5% Ga-123-P cal, depicting wound closure after 24 h.

FIG. 33D shows an image of an in vitro assay using 1% Ga-123-P cal, depicting wound closure after 24 h.

FIG. 33E shows an image of an in vitro assay using 0% Ga-123-F cal, depicting wound closure after 24 h.

FIG. 33F shows an image of an in vitro assay using 0.2% Ga-123-F cal, depicting wound closure after 24 h.

FIG. 33G shows an image of an in vitro assay using 0.5% Ga-123-F cal, depicting wound closure after 24 h.

FIG. 33H shows an image of an in vitro assay using 1% Ga-123-F cal, depicting wound closure after 24 h.

### DETAILED DESCRIPTION

The present invention arises from the inventors' work in attempting to overcome the problems associated with the prior art.

In accordance with a first aspect of the invention, there is provided a fibre comprising or consisting of a porous phosphate-based glass (PBG).

Advantageously, the PBG fibres of the first aspect may be produced using electrospinning. This can produce fibres of smaller diameter and increased flexibility compared to prior art fibres. This enables the fibres to exhibit improved conformability to complex wound topography. Moreover, temperature-sensitive molecules can be incorporated into the fibres, instead of high temperature melts. The ability to include thermal-sensitive molecules is innovative and opens up significant new opportunities for therapy.

Preferably, the pores are aligned in the phosphate-based glass.

The fibre may have an average diameter of less than 100 μm, less than 50 μm, less than 25 μm, less than 10 μm, less than 5 μm, less than 3 μm, less than 2 μm or less than 1.8 μm. The fibre may have an average diameter of at least 0.1 μm, at least 0.25 μm, at least 0.5 μm, at least 0.75 μm, at least 1 μm, at least 1.2 μm, at least 1.4 μm or at least 1.5 μm. The fibre may have an average diameter of between 0.1 and 100 μm, between 0.25 and 50 μm, between 0.5 and 25 μm, between 0.75 and 10 μm, between 1 and 5 μm, between 1.2 and 3 μm, between 1.4 and 2 μm or between 1.5 and 1.8 μm. The average diameter of the fibre may be the median diameter (D (50)) of the fibre. The average diameter of the fibre may be calculated from analysis of one or more SEM images of the fibre or a plurality of fibres. For instance, the average diameter of the fibre may be calculated by measuring a plurality of diameters of a fibre or a plurality of fibres in the one or more SEM images and calculating the average of the measured plurality of diameters. The average diameter of the fibre may be calculated using ImageJ software.

Preferably, the phosphate-based glass comprises an alkaline earth metal oxide. The alkaline earth metal oxide may be one or more of beryllium oxide, magnesium oxide, calcium oxide, strontium oxide and/or barium oxide. In one embodiment, the phosphate-based glass preferably comprises between 5 and 70 mol %, between 10 and 60 mol % or between 20 and 55 mol % alkaline earth metal oxide, more preferably between 25 and 50 mol % or between 30 and 45 mol % alkaline earth metal oxide and most preferably between 35 and 40 mol % alkaline earth metal oxide. In another embodiment, the phosphate-based glass preferably comprises between 5 and 70 mol %, between 10 and 65 mol % or between 25 and 60 mol % alkaline earth metal oxide, more preferably between 30 and 55 mol % or between 35 and 50 mol % alkaline earth metal oxide and most preferably between 40 and 45 mol % alkaline earth metal oxide.

The phosphate-based glass may comprise between 0.5 and 30 atom %, between 1 and 25 atom % or between 2 and 20 atom % the alkaline earth metal, and more preferably between 3 and 17.5 atom %, between 5 and 15 atom % or between 8 and 13 atom % the alkaline earth metal. In one embodiment, the phosphate-based glass may comprise between 0.5 and 30 atom %, between 1 and 25 atom % or between 2 and 20 atom % calcium, and more preferably between 3 and 17.5 atom %, between 5 and 15 atom % or between 8 and 13 atom % calcium. In an alternative embodiment, the phosphate-based glass may comprise between 0.5 and 30 atom %, between 1 and 25 atom % or between 2 and 20 atom % magnesium, and more preferably between 3 and 17.5 atom %, between 5 and 15 atom % or between 8 and 13 atom % magnesium.

In some embodiments, the phosphate-based glass comprises calcium oxide. In some embodiments, the phosphate-based glass comprises magnesium oxide. In some embodiments, the alkaline earth metal oxide consists of magnesium oxide. In one preferred embodiment, the phosphate-based glass comprises calcium oxide and magnesium oxide.

The inventors believe that the combination of calcium oxide and magnesium oxide is novel and inventive per se.

Accordingly, in accordance with a second aspect, there is provided a phosphate-based glass comprising a combination of calcium oxide and magnesium oxide.

Advantageously, the inventors have found that phosphate-based glasses comprising magnesium oxide in combination with calcium oxide offer improved wound healing properties.

Preferably, the phosphate-based glass is porous.

The phosphate-based glass may have an average pore diameter of less than 1,000 nm, less than 900 nm, less than 800 nm, less than 700 nm, less than 600 nm, less than 500 nm, less than 400 nm, less than 300 nm, less than 250 nm, less than 200 nm, less than 150 nm or less than 100 nm. The phosphate-based glass may have an average pore diameter of at least 50 nm, at least 100 nm, at least 150 nm, at least 200 nm, at least 250 nm, at least 300 nm, at least 400 nm, at least 500 nm, at least 600 nm, at least 700 nm, at least 800 nm or at least 900 nm. The phosphate-based glass may have an average pore diameter of between 25 and 1,000 nm, between 50 and 900 nm, between 75 and 800 nm, between 100 and 700 nm, between 125 and 600 nm, between 150 and 500 nm, between 175 and 400 nm, between 200 and 300 nm or between 225 and 250 nm. The average pore diameter may be the median pore diameter (D (50)). The average pore diameter may be calculated from analysis of one or more SEM images of the phosphate-based glass. For instance, the average pore diameter may be calculated by measuring a plurality of diameters of pores in the one or more SEM images and calculating the average of the measured plurality of diameters. The average pore diameter of the fibre may be calculated using ImageJ software.

Preferably, the phosphate-based glass comprises between 2.5 and 50 mol % or between 5 and 45 mol % calcium oxide, more preferably between 10 and 40 mol % or between 15 and 35 mol % calcium oxide and most preferably between 20 and 30 mol % calcium oxide. The phosphate-based glass may comprise between 0.5 and 25 atom %, between 1 and 20 atom %, between 2 and 15 atom % or between 3 and 10 atom % calcium, more preferably between 4 and 7.5 atom % or between 4.5 and 6 atom % calcium.

Preferably, the phosphate-based glass comprises between 1 and 50 mol %, between 2 and 40 mol %, between 4 and 30 mol %, or between 6 and 25 mol % magnesium oxide, more preferably between 7 and 20 mol % or between 8.5 and 17.5 mol % magnesium oxide and most preferably between 10 and 15 mol % magnesium oxide. The phosphate-based glass may comprise between 0.1 and 15 atom %, between 0.5 and 10 atom %, between 1 and 5 atom % or between 1.5 and 4 atom % magnesium, more preferably between 2 and 3.5 atom % or between 1.5 and 3 atom % magnesium.

Preferably, the molar ratio of calcium oxide to magnesium oxide is between 20:1 and 1:4, between 10:1 and 1:2 or between 8:1 and 1:1.5 more preferably between 6:1 and 1:1, between 4:1 and 1.25:1 or between 2.5:1 and 1.5:1, and most preferably is about 1:1. The atomic ratio of calcium to magnesium may be understood to be the same as the molar ratio of calcium oxide to magnesium oxide.

Preferably, the phosphate-based glass comprises phosphorus pentoxide. Preferably, the phosphate-based glass comprises between 10 and 90 mol % phosphorus pentoxide, more preferably between 20 and 80 mol %, between 25 and 70 mol % or between 30 and 60 mol % phosphorus pentoxide, and most preferably between 35 and 55 mol % or between 40 and 50 mol % phosphorus pentoxide. The phosphate-based glass may comprise between 5 and 40 atom %, between 10 and 35 atom %, between 15 and 30 atom % or between 17.5 and 25 atom % phosphorous, more preferably between 18 and 23 atom %, between 19 and 22.5 atom % or between 20 and 21.5 atom % phosphorous.

The phosphate-based glass may comprise between 40 and 90 atom %, between 50 and 80 atom % or between 55 and 75 atom % oxygen, more preferably between 60 and 70 atom %, between 62 and 68 atom % or between 64 and 66 atom % oxygen.

Preferably, the phosphate-based glass comprises an alkali metal oxide and/or a group 13 oxide. The alkali metal oxide may be lithium oxide, sodium oxide, potassium oxide or rubidium oxide. The group 13 oxide may be boron oxide.oxide or borate. Preferably, the alkali metal oxide is sodium oxide. Preferably, the phosphate-based glass comprises between 1 and 50 mol % alkali metal oxide and/or a group 13 oxide, more preferably between 2.5 and 30 mol % or between 5 and 20 mol % alkali metal oxide and/or a group 13 oxide and most preferably between 10 and 15 mol % alkali metal oxide and/or a group 13 oxide. In other embodiments, the phosphate-based glass preferably comprises between 1 and 30 mol % alkali metal oxide and/or a group 13 oxide, more preferably between 2.5 and 15 mol % or between 4 and 11 mol % alkali metal oxide and/or a group 13 oxide and most preferably between 5 and 10 mol % alkali metal oxide and/or a group 13 oxide. The phosphate-based glass may comprise between 0.5 and 20 atom %, between 1 and 15 atom %, between 2 and 12.5 atom % or between 2.5 and 10 atom % alkali metal oxide and/or a group 13 oxide, more preferably between 3 and 8 atom %, between 4 and 7 atom % or between 4.5 and 6 atom % alkali metal oxide and/or a group 13 oxide.

In some embodiments, the phosphate-based glass does not comprise boron oxide or borate.

Optionally, the phosphate-based glass comprises a therapeutic agent. The therapeutic agent may be an antimicrobial agent and/or an antioxidant.

The therapeutic agent may comprise a therapeutic metal or therapeutic metal cation. In some embodiments, the therapeutic metal or therapeutic metal cation may comprise or be an antimicrobial metal or antimicrobial metal cation. A therapeutic metal or therapeutic metal cation may be understood to be a metal or metal cation, respectively, which has one or more therapeutic properties. Similarly, an antimicrobial metal or antimicrobial metal cation may be understood to be a metal or metal cation, respectively, which has antimicrobial properties.

The therapeutic metal or therapeutic metal cation may be a transition metal, a p-block metal or a cation thereof. The therapeutic metal or metal cation may be or comprise copper, zinc, strontium, silver, gallium, cerium, titanium, cobalt, manganese, iron, potassium or a cation thereof. The therapeutic metal cation may be a 1+, 2+, 3+ or 4+ cation. The therapeutic metal cation may be provided in the form of a metal oxide, a metal acetate, a metal nitrate, a metal chloride or a metal sulphate, e.g. silver oxide, copper oxide, zinc oxide, strontium oxide, titanium oxide, cobalt oxide, manganese oxide, iron oxide, potassium oxide, gallium oxide, cerium oxide, copper acetate, zinc acetate, strontium acetate, silver acetate or gallium acetate.

In some preferred embodiments, the therapeutic metal or metal cation silver or a cation thereof. In some embodiments, the therapeutic metal cation is a Ag⁺ cation.

In alternative preferred embodiments, the therapeutic metal or metal cation may be or comprise gallium or cerium or a cation thereof. In some embodiments, the therapeutic metal cation is a Ce ion (e.g. a Ce³⁺ ion) or Ga ion (e.g. a Ga³⁺ ion).

In some preferred embodiments, the therapeutic metal cation may be provided in the form of silver oxide. In alternative preferred embodiments, the therapeutic metal cation may be provided in the form of gallium oxide or cerium oxide. Preferably, the therapeutic metal cation may be provided in the form of gallium (III) oxide or cerium (III) oxide.

In some embodiments, the phosphate-based glass comprises between 0.001 and 20 mol % of the therapeutic agent, more preferably between 0.01 and 15 mol % or between 0.05 and 10 mol % of the therapeutic agent and most preferably between 0.1 and 7.5 mol %, between 0.4 and 5 mol %, between 0.6 and 3 mol % or between 0.8 and 1.5 mol % of the therapeutic agent. In some embodiments, the phosphate-based glass comprises between 0.001 and 20 mol % of the therapeutic metal or therapeutic metal cation, more preferably between 0.01 and 15 mol % or between 0.05 and 10 mol % of the therapeutic metal or therapeutic metal cation and most preferably between 0.1 and 7.5 mol %. In some embodiments, the phosphate-based glass comprises between 0.2 and 5 mol %, between 0.4 and 3 mol %, between 0.6 and 2 mol %, between 0.7 and 1.5 mol %, between 0.75 and 1.3 mol % or between 0.8 and 1.2 mol % of the therapeutic metal or therapeutic metal cation. In some embodiments, the phosphate-based glass comprises between 1 and 5 mol %, between 2 and 4 mol %, between 2.5 and 3.5 mol % or between 2.75 and 3.25 mol % of the therapeutic metal or therapeutic metal cation. In some embodiments, the phosphate-based glass comprises between 1 and 7 mol %, between 3 and 6 mol %, between 4.5 and 5.5 mol % or between 4.75 and 5.25 mol % of the therapeutic metal or therapeutic metal cation. It may be appreciated that when discussing the mol % of the therapeutic cation, the percentage may refer to the mol % of any compound comprising the therapeutic cation. For instance, if the therapeutic cation was provided as an oxide, then the percentages would be understood to define the molar percentage of the oxide of the oxide.

In some embodiments, the phosphate-based glass comprises between 0.001 and 20 atom % of the therapeutic metal or therapeutic metal cation, more preferably between 0.005 and 15 atom % or between 0.001 and 10 atom % of the therapeutic metal or therapeutic metal cation and most preferably between 0.01 and 5 atom %. In some embodiments, the phosphate-based glass comprises between 0.01 and 1.5 atom %, between 0.05 and 1 atom %, between 0.1 and 0.8 atom %, between 0.2 and 0.6 atom %, between 0.25 and 0.5 atom % or between 0.3 and 0.4 atom % of the therapeutic metal or therapeutic metal cation. In some embodiments, the phosphate-based glass comprises between 0.01 and 2.5 atom %, between 0.05 and 2 atom %, between 0.1 and 1.5 atom %, between 0.3 and 1 atom %, between 0.4 and 0.8 atom % or between 0.5 and 0.6 atom % of the therapeutic metal or therapeutic metal cation. In some embodiments, the phosphate-based glass comprises between 1 and 3 atom %, between 1.25 and 2.5 atom %, between 1.5 and 2 atom % or between 1.7 and 1.8 atom % of the therapeutic metal or therapeutic metal cation. In some embodiments, the phosphate-based glass comprises between 1.5 and 4 atom %, between 2 and 3 atom %, between 2.2 and 2.75 atom % or between 2.4 and 2.5 atom % of the therapeutic metal or therapeutic metal cation.

Alternatively, or additionally, the therapeutic agent may comprise an organic molecule. The organic molecule may be an active pharmaceutical ingredient (API), a polymer and/or a protein. The polymer may be a polysaccharide, a biopolymer or a synthetic polymer. The polysaccharide may be chitosan or alginate. The biopolymer may be gelatine, collagen or zein. The synthetic polymer may be poly(methyl methacrylate) (PMMA), polycaprolactone (PCL), or polyvinyl alcohol (PVA). The organic molecule may be configured to enhance biological activity. The organic molecule, and preferably the API, may be a small molecule. In some embodiments, the organic molecule may be hyaluronic acid or a growth factor. The growth factor may be vascular endothelial growth factor (VEGF), epidermal growth factor (EGF), fibroblast growth factor (FGF), connective tissue growth factor (CTGF), platelet-derived growth factor (PDGF) or transforming growth factor beta (TGF-β).

The organic molecule, and preferably the polymer, may have a molecule weight of between 10,000 and 10,000,000 g/mol, between 50,000 and 7,500,000 g/mol, between 100,000 and 5,000,000 g/mol, between 500,000 and 4,000,000 g/mol, between 1,000,000 and 3,000,000 g/mol, between 1, 500,000 and 2,500,000 g/mol or between 2,000,000 and 2,400,000 g/mol.

Alternatively, the organic molecule may have a molecular weight of less than 500,000 g/mol, less than 100,000 g/mol, less than 50,000 g/mol, less than 10,000 g/mol, more preferably less than 5,000 g/mol or less than 1,000 g/mol. The organic molecule may be understood to be a small molecule if it has a molecular weight of less than 2,000 g/mol, more preferably less than 1,500 or less than 1,000 g/mol, and most preferably less than 900 g/mol.

In some embodiments, the phosphate-based glass comprises between 0.0001 and 5 wt % of the organic molecule, more preferably between 0.001 and 2 wt % or between 0.005 and 1 wt % of the organic molecule, and most preferably between 0.01 and 0.5 wt %, between 0.04 and 0.2 wt %, between 0.06 and 0.15 wt % or between 0.08 and 0.1 wt % of the organic molecule.

The therapeutic agent may be disposed in the pores of the phosphate-based glass.

The antioxidant may be an essential oil. The essential oil may be extracted from a plant. The plant may be from the Anonaceae family, the Apiaceae family, the Asteraceae family, the Ericaceae family, the Geraniaceae family, the Lamiaceae family, the Lauraceae family, the Myrtaceae family, the Oleaceae family, the Poaceae family, the Pinaceae family, the Rutaceae family, the Verbenaceae family, or the Zingiberaceae family. The therapeutic agent may be a cedarwood essential oil (e.g. cedarwood atlas), a chamomile essential oil, a cardamom essential oil, a clove essential oil (e.g. clove bud), a eucalyptus essential oil (e.g. Eucalyptus rakiata), a jasmine essential oil (e.g. jasmine absolute), a myrtle essential oil (e.g. lemon myrtle), a neroli essential oil (i.e. essential oil from the bitter orange tree), a niaouli essential oil, a petitgrain essential oil (i.e. essential oil from the bitter orange tree), a ravensara essential oil, a geranium rose essential oil, a rosemary essential oil, a rosewood essential oil, a marjoram essential oil (e.g. sweet marjoram), a tea-tree essential oil, a thyme essential oil, a basil essential oil (e.g. tropical basil), a vetiver essential oil (e.g. bourbon vetiver), a ylang ylang essential oil a spruce essential oil (e.g. black spruce), an Ammi visnaga essential oil, a rose essential oil, a lantana essential oil, a verbena essential oil (e.g. lemon verbena) or a Labrador tea essential oil. The therapeutic agent may be a clove essential oil.

In some embodiments, the phosphate-based glass comprises between 0.001 and 20 wt % of the antioxidant, more preferably between 0.01 and 15 wt % or between 0.1 and 10 wt % of the antioxidant, and most preferably between 0.5 and 7.5 wt %, between 1 and 5 wt %, between 2 and 4 wt % or between 2.5 and 3.5 wt % of the antioxidant.

Preferably, the phosphate-based glass is amorphous. A phosphate-based glass may be considered to be amorphous if a wide-angle x-ray powder diffraction spectrum of the glass has no Bragg peaks.

In accordance with a third aspect, there is provided a method of producing a fibre comprising or consisting of a porous phosphate-based glass, the method comprising:
contacting a phosphate, a surfactant and an alkaline earth metal cation in a reaction solvent to provide a reaction solution; and
electrospinning at least a portion of the reaction solution to provide the fibre comprising or consisting of a porous phosphate-based glass.

The method of the third aspect may provide the fibre of the first aspect.

The reaction solvent may be or comprise water. In some embodiments, the reaction solvent may comprise an alcohol. The alcohol may be or comprise a C₁₋₆ alcohol, more preferably a C₁₋₄ alcohol or most preferably a C₁₋₃ alcohol. The alcohol may be or comprise methanol, ethanol, propanol and/or butanol. In some embodiments, the alcohol may be ethanol.

The reaction solvent may comprise or consist of water and the alcohol. Preferably, the reaction solvent comprises less than 5 vol % alcohol or less than 2.5 vol % alcohol. Preferably, the reaction solvent comprises between 0 and 10 vol % alcohol, between 0.5 and 5 vol % alcohol, between 1 and 4 vol % alcohol or between 2 and 3 vol % alcohol.

Advantageously, the inventors have found that the addition of alcohol increases the porosity of the glass.

Contacting the phosphate, the surfactant and the alkaline earth metal cation may comprise:
providing a first solution comprising the phosphate and a first solvent;
providing a second solution comprising the surfactant, the alkaline earth metal cation and a second solvent; and
combining the first and second solutions to provide the reaction solution.

Accordingly, the reaction solvent may comprise a mixture of the first and second solvents.

Providing a second solution may comprise dissolving the surfactant in the second solvent, and subsequently dissolving the alkaline earth metal cation in the second solvent.

Alternatively, providing the second solution may comprise:
dissolving the surfactant in a solvent to create a surfactant solution;
dissolving the alkaline earth metal cation in a solvent to create an alkaline earth metal cation solution; and
subsequently combining the surfactant solution and the metal cation solution to provide the second solution.

The solvent which the surfactant is dissolved in may be the same or different to the solvent which the alkaline earth metal cation is dissolved in. The solvent the surfactant is dissolved and the solvent which the alkaline earth metal cation is dissolved in may both be the second solvent. Alternatively, the combination of the solvent the surfactant is dissolved and the solvent which the alkaline earth metal cation is dissolved in may provide the second solvent.

The second solvent may be or comprise water. In some embodiments, the second solvent may be or comprise an alcohol. The alcohol may be as defined above. The second solvent may comprise or consist of water and the alcohol. It may be appreciated that the amount of alcohol in the second solvent may be selected to give an alcohol concentration in the reaction solvent as defined above.

The second solvent may comprise less than 20 vol % alcohol or less than 10 vol % alcohol. In some embodiments, the second solvent comprises between 0 and 20 vol % alcohol, between 1 and 10 vol % alcohol, between 1 and 8 vol % alcohol, between 4 and 6 vol % alcohol or between 4.5 and 5.5 vol % alcohol.

The reaction solution may comprise the alkaline earth metal cation at a concentration of between 0.001 and 20 M, between 0.01 and 15 M, between 0.05 and 10 M or between 0.1 and 7.5 M. The reaction solution may comprise between 0.2 and 5 M, between 0.4 and 3 M, between 0.6 and 1.5 M or between 0.75 and 1.25 M. Alternatively, the reaction solution may comprise the alkaline earth metal cation at a concentration of between 0.1 and 1 M, between 0.15 and 0.75 M, between 0.2 and 0.5 M or between 0.25 and 0.3 M.

The concentration of the alkaline earth metal cation in the second solution may be selected depending upon the desired concentration of the alkaline earth metal cation in the reaction solution. The second solution may comprise the alkaline earth metal cation at a concentration of between 0.001 and 25 M, between 0.01 and 20 M, between 0.05 and 15 M, between 0.1 and 10 M, between 0.25 and 7.5 M. The second solution may comprise the alkaline earth metal cation at a concentration of between 0.5 and 5 M, between 1 and 3 M or between 1.5 and 2.5 M. Alternatively, the second solution may comprise the alkaline earth metal cation at a concentration of between 0.01 and 1 M, between 0.1 and 0.75 M, between 0.2 and 0.5 M, between 0.25 and 0.4 M or between 0.3 and 0.35 M.

The method may comprise contacting the phosphate and the alkaline earth metal cation at an atomic ratio of between 1:10 and 50:1, between 1:5 and 25:1, between 1:2 and 10:1, between 0.75:1 and 5:1, between 1:1 and 3:1, between 1.5:1 and 2.5:1 or between 1.75:1 and 2.25:1 phosphorous to alkaline earth metal. In some embodiments, the method may comprise contacting the phosphate and the alkaline earth metal cation at an atomic ratio of about 2:1 phosphorous to alkaline earth metal.

The alkaline earth metal cation may be provided with a counter ion. The counter ion may be a nitrate, an acetate, a sulphate or a halide. The halide may be a chloride.

The alkaline earth metal cation may be or comprise a beryllium cation, a magnesium cation, a calcium cation, a strontium cation and/or a barium cation. In some embodiments, the alkaline earth metal cation may be or comprise a calcium cation. In some embodiments, the alkaline earth metal cation may be or comprise a magnesium cation. In some embodiments, the alkaline earth metal cation may comprise a combination of two or more alkaline earth metal cations, preferably a calcium cation and a magnesium cation.

Accordingly, contacting the phosphate, the surfactant and the alkaline earth metal cation may comprise:
providing a first solution comprising the phosphate and the first solvent;
providing a second solution comprising the surfactant, a calcium cation, a magnesium cation and the second solvent; and
combining the first and second solutions to provide the reaction solution.

The method may comprise contacting the phosphate, the surfactant and the alkaline earth metal cation with a therapeutic agent in the reaction solution. The therapeutic agent may comprise a therapeutic metal or therapeutic metal cation. The therapeutic metal or therapeutic metal cation may be as defined above. The therapeutic metal cation may be provided with a counter ion. The counter ion may be a nitrate, an oxide, an acetate or a halide. The halide may be a chloride.

The atomic ratio of the therapeutic metal or therapeutic metal cation to the combination of the phosphorous and the alkaline earth metal cation may be between 0.001:99.999 and 20:80, between 0.01:99.99 and 10:90, between 0.05:99.95 and 10:90, between 0.1:99.9 and 7.5:92.5, between 0.4:99.6 and 5:95, between 0.6:99.4 and 3:97 or between 0.8:99.2 and 1.5:98.5.

The reaction solution may comprise the therapeutic metal or therapeutic metal cation at a concentration of between 0.00001 and 1 M, between 0.0001 and 0.5 M or between 0.001 and 0.2 M, between 0.005 and 0.1 M, between 0.008 and 0.08 M, between 0.01 and 0.06 M or between 0.015 and 0.055 M.

The method may comprise stirring the reaction solution. The method may comprise stirring the reaction solution for at least 5 minutes, at least 10 minutes, at least 20 minutes, at least 30 minutes, at least 45 minutes or at least 1 hour.

The method may comprise allowing phase separation to occur in the reaction solution to provide a coacervate phase and a supernatant phase. The method may comprise allowing the reaction solution to settle, and to thereby allow phase separation to occur. The method may comprise allowing the reaction solution to settle for at least 5 minutes, at least 10 minutes, at least 20 minutes, at least 30 minutes, at least 45 minutes or at least 1 hour. Preferably, the method comprises allowing the reaction solution to settle for at least 2 hours, at least 4 hours, at least 6 hours, at least 12 hours or at least 24 hours.

The method may comprise separating the coacervate phase and the supernatant phase.

The coacervate phase may be the portion of reaction solution which is electrospun. Accordingly, the method may comprise electrospinning the coacervate phase to provide the fibre comprising or consisting of a porous phosphate-based glass.

Electrospinning at least a portion of the reaction solution may comprise feeding the at least a portion of the reaction solution flow through a capillary tip and then collecting it on a collector.

The capillary may have an internal diameter of between 0.01 and 10 mm, between 0.1 and 5 mm, between 0.2 and 3 mm, between 0.3 and 2 mm, between 0.4 and 1.75 mm, between 0.5 and 1.5 mm, between 0.6 and 1.25 mm, between 0.7 and 1 mm or between 0.8 and 0.9 mm.

The method may comprise applying a potential difference between the capillary tip and the collector. The potential difference may be at least 0.1 kV, at least 1 kV, at least 5 kV, at least 10 kV, at least 12.5 kV, at least 15 kV or at least 16 kV. The potential difference may be between 0.1 and 100 kV, between 1 and 75 kV, between 5 and 50 kV, between 10 and 30 kV, between 12.5 and 25 kV, between 15 and 20 kV or between 16 and 18 kV.

The difference between the capillary tip and the collector may be between 1 and 50 cm, between 2.5 and 40 cm, between 5 and 30 cm, between 10 and 20 cm, between 12 and 17 cm or between 14 and 15 cm.

The method may comprise feeding at least a portion of the reaction solution through the capillary at a flow rate of between 0.1 and 50 ml/h, between 0.25 and 20 ml/h, between 0.5 and 10 ml/h, between 0.75 and 7.5 ml/h, between 1 and 5 ml/h, between 1.25 and 3 ml/h, between 1.5 and 2.5 ml/h, between 1.75 and 2.25 ml/h or between 1.9 and 2.1 ml/h.

The method may comprise electrospinning at least a portion of the reaction solution at a temperature between-25 and 100° C., between-10 and 75° C., between 0 and 50° C., between 5 and 40° C., between 1° and 30° C. or between 15 and 25° C. In a preferred embodiment, the method comprises electrospinning at least a portion of the reaction solution at room temperature.

The method may comprise calcinating the fibre. The method may comprise calcinating the fibre subsequently to electrospinning at least a portion of the reaction solution.

Calcinating the fibre may comprise holding the fibre at an elevated temperature. The elevated temperature may be at least 100° C., at least 150° C., at least 200° C., at least 250° C. or at least 275° C. The elevated temperature may be between 10° and 500° C., between 15° and 450° C., between 200 and 400° C., between 25° and 350° C. or between 275 and 325° C. The elevated temperature may be between 10° and 500° C., between 150 and 450° C., between 175 and 425° C., between 20° and 400° C. or between 325 and 375° C.

Calcinating the fibre may comprise raising the temperature of the fibre to the elevated temperature at a predefined rate. The predefined rate may be between 0.05 and 15° C./min, between 0.1 and 10° C./min, between 0.25 and 7.5° C./min, between 0.5 and 5° C./min, between 0.6 and 4° C./min, between 0.7 and 3° C./min, between 0.8 and 2° C./min or between 0.9 and 1.5° C./min. Preferably, the predefined rate is about 1° C./min.

The method may comprise contacting the fibre with a therapeutic agent. The method may comprise contacting the fibre with a solution comprising the therapeutic agent. The solution may comprise the therapeutic agent at a concentration of less than 50 wt %, less than 40 wt %, less than 30 wt %, less than 20 wt %, less than 10 wt %, less than 7.5 wt % or less than 5 wt %. The therapeutic agent may comprise an organic molecule. The organic molecule may be as defined above. The method may comprise contacting the fibre with a therapeutic agent, preferably an organic molecule, after calcinating the fibre. Advantageously, this allows the fibre to be loaded with a temperature sensitive molecule.

In accordance with a fourth aspect, there is provided a method of producing a phosphate-based glass comprising a combination of calcium oxide and magnesium oxide, the method comprising:
contacting a phosphate, a surfactant, a calcium cation and a magnesium cation in a reaction solvent to provide a reaction solution; and
calcinating at least a portion of the reaction solution to provide the phosphate-based glass comprising a combination of calcium oxide and magnesium oxide.

The method of the fourth aspect may produce the glass of the second aspect.

The reaction solvent may comprise or consist of water and the alcohol. The inventors have found that the addition of alcohol increases porosity.

In some embodiments, the reaction solvent may comprise less than 10 vol % alcohol or less than 5 vol % alcohol. In some embodiments, the reaction solvent comprises between 0 and 10 vol % alcohol, between 0.5 and 5 vol % alcohol, between 1 and 4 vol % alcohol or between 2 and 3 vol % alcohol.

In another embodiment, the reaction solvent comprises between 0 and 75 vol % alcohol, between 0.5 and 50 vol % alcohol, between 1 and 40 vol % alcohol, between 2 and 30 vol % alcohol, between 4 and 20 vol % alcohol, between 6 and 15 vol % alcohol, between 7.5 and 12.5 vol % alcohol or between 9 and 11 vol % alcohol. In particular, the inventors have found that a concentration of about 10 vol % alcohol produces powders with the greatest porosity.

Contacting the phosphate, the surfactant, the calcium cation and the magnesium cation may comprise:
providing a first solution comprising the phosphate and a first solvent;
providing a second solution comprising the surfactant, the calcium cation, the magnesium cation and a second solvent; and
combining the first and second solutions to provide the reaction solution.

Accordingly, the reaction solvent may comprise a mixture of the first and second solvents.

Providing a second solution may comprise dissolving the surfactant in the second solvent, and subsequently dissolving the calcium cation and magnesium cation in the second solvent.

The second solvent may be or comprise water. In some embodiments, the second solvent may be or comprise an alcohol. The alcohol may be as defined above. The second solvent may comprise or consist of water and the alcohol. It may be appreciated that the amount of alcohol in the second solvent may be selected to give an alcohol concentration in the reaction solvent as defined above.

In some embodiments, the second solvent may comprise less than 20 vol % alcohol or less than 10 vol % alcohol. In some embodiments, the second solvent comprises between 0 and 20 vol % alcohol, between 1 and 10 vol % alcohol, between 1 and 8 vol % alcohol, between 4 and 6 vol % alcohol or between 4.5 and 5.5 vol % alcohol.

In another embodiment, the second solvent comprises between 0 and 100 vol % alcohol, between 1 and 80 vol % alcohol, between 2 and 60 vol % alcohol, between 4 and 50 vol % alcohol, between 8 and 40 vol % alcohol, between 12 and 30 vol % alcohol, between 15 and 25 vol % alcohol or between 18 and 22 vol % alcohol.

The calcium cation and/or the magnesium cation may be provided with a counter ion. The counter ion may be as defined above.

The combined concentration of the calcium and magnesium cations in the reaction solution may be between 0.001 and 20 M, between 0.01 and 15 M, between 0.05 and 10 M, between 0.1 and 7.5 M, between 0.2 and 5 M, between 0.4 and 3 M, between 0.6 and 1.5 M or between 0.75 and 1.25 M.

The combined concentration of the calcium and magnesium cations in the second solution may be selected depending upon the desired concentration in the reaction solution. Accordingly, the combined concentration of the calcium and magnesium cations in the second solution may be between 0.001 and 25 M, between 0.01 and 20 M, between 0.05 and 15 M, between 0.1 and 10 M, between 0.25 and 7.5 M, between 0.5 and 5 M, between 1 and 3M or between 1.5 and 2.5 M.

The reaction solution may comprise the calcium cation at a concentration of between 0.0001 and 10 M, between 0.005 and 7.5 M, between 0.01 and 5 M, between 0.05 and 3 M, between 0.1 and 2 M, between 0.2 and 1 M, between 0.3 and 0.8 M or between 0.4 and 0.6 M.

The concentration of the calcium cation in the second solution may be selected depending upon the desired concentration of the calcium cation in the reaction solution. The second solution may comprise the calcium cation at a concentration of between 0.001 and 20 M, between 0.01 and 15 M, between 0.05 and 10 M, between 0.1 and 7.5 M, between 0.2 and 5 M, between 0.4 and 3 M, between 0.6 and 1.5 M or between 0.75 and 1.25 M.

The reaction solution may comprise the magnesium cation at a concentration of between 0.0001 and 10 M, between 0.005 and 7.5 M, between 0.01 and 5 M, between 0.05 and 3 M, between 0.1 and 2 M, between 0.2 and 1 M, between 0.3 and 0.8 M or between 0.4 and 0.6 M.

The concentration of the magnesium cation in the second solution may be selected depending upon the desired concentration of the magnesium cation in the reaction solution. The second solution may comprise the magnesium cation at a concentration of between 0.001 and 20 M, between 0.01 and 15 M, between 0.05 and 10 M, between 0.1 and 7.5 M, between 0.2 and 5 M, between 0.4 and 3 M, between 0.6 and 1.5 M or between 0.75 and 1.25 M.

The atomic ratio of the calcium cation and the magnesium cation in the second solution and/or the reaction solution may be between 1:20 and 20:1, between 1:10 and 10:1, between 1:5 and 5:1, between 1:3 and 3:1, between 1:2 and 2:1, between 1:1.5 and 1.5:1 or between 1:1.25 and 1.25:1. In some embodiments, the atomic ratio of the calcium cation and the magnesium cation is about 1:1.

The method may comprise contacting the phosphate and the calcium and magnesium cations at an atomic ratio of between 1:10 and 50:1, between 1:5 and 25:1, between 1:2 and 10:1, between 0.75:1 and 5:1, between 1:1 and 3:1, between 1.5:1 and 2.5:1 or between 1.75:1 and 2.25:1 phosphorous to the combination of calcium and magnesium. In some embodiments, the method may comprise contacting the phosphate and the calcium and magnesium cations at an atomic ratio of about 2:1 phosphorous to the combination of calcium and magnesium.

The method may comprise contacting the phosphate and the calcium cation at an atomic ratio of between 1:20 and 50:1, between 1:10 and 25:1, between 1:5 and 20:1, between 1:2 and 15:1, between 1:1 and 10:1, between 2:1 and 7.5:1 or between 3:1 and 5:1 phosphorous to calcium. In some embodiments, the method may comprise contacting the phosphate and the calcium cation at an atomic ratio of about 4:1 phosphorous to calcium.

The method may comprise contacting the phosphate and the magnesium cation at an atomic ratio of between 1:20 and 50:1, between 1:10 and 25:1, between 1:5 and 20:1, between 1:2 and 15:1, between 1:1 and 10:1, between 2:1 and 7.5:1 or between 3:1 and 5:1 phosphorous to magnesium. In some embodiments, the method may comprise contacting the phosphate and the magnesium cation at an atomic ratio of about 4:1 phosphorous to magnesium.

The method may comprise contacting the phosphate, the surfactant, the calcium cation and the magnesium cation with a therapeutic agent in the reaction solution. The therapeutic agent may comprise a therapeutic metal or therapeutic metal cation. The therapeutic metal or therapeutic metal cation may be as defined above. The therapeutic metal cation may be provided with a counter ion. The counter ion may be a nitrate, oxide, acetate or halide. The halide may be a chloride.

The atomic ratio of the therapeutic metal or therapeutic metal cation to the combination of the phosphorous, the calcium cation and the magnesium cation may be between 0.001:99.999 and 20:80, between 0.01:99.99 and 10:90, between 0.05:99.95 and 10:90, between 0.1:99.9 and 7.5:92.5, between 0.4:99.6 and 5:95, between 0.6:99.4 and 3:97 or between 0.8:99.2 and 1.5:98.5.

The method may comprise stirring the reaction solution. The method may comprise stirring the reaction solution for at least 5 minutes, at least 10 minutes, at least 20 minutes, at least 30 minutes, at least 45 minutes or at least 1 hour.

The method may comprise allowing phase separation to occur in the reaction solution to provide a coacervate phase and a supernatant phase. The method may comprise allowing the reaction solution to settle, and to thereby allow phase separation to occur. The method may comprise allowing the reaction solution to settle for at least 5 minutes, at least 10 minutes, at least 20 minutes, at least 30 minutes, at least 45 minutes or at least 1 hour. Preferably, the method comprises allowing the reaction solution to settle for at least 2 hours, at least 4 hours, at least 6 hours, at least 12 hours or at least 24 hours.

The method may comprise separating the coacervate phase and the supernatant phase.

The coacervate phase may be the portion of the reaction solution which is calcination. Accordingly, the method may comprise calcinating the coacervate phase to provide the phosphate-based glass comprising a combination of calcium oxide and magnesium oxide.

Calcinating the at least a portion of the reaction solution may comprise holding the at least a portion of the reaction solution at an elevated temperature. The elevated temperature may be at least 100° C., at least 150° C., at least 200° C., at least 250° C. or at least 275° C. The elevated temperature may be between 100 and 500° C., between 150 and 450° C., between 20° and 400° C., between 25° and 350° C. or between 275 and 325° C.

Calcinating the at least a portion of the reaction solution may comprise raising the temperature of the at least a portion of the reaction solution to the elevated temperature at a predefined rate. The predefined rate may be between 0.05 and 15° C./min, between 0.1 and 10° C./min, between 0.25 and 7.5° C./min, between 0.5 and 5° C./min, between 0.6 and 4° C./min, between 0.7 and 3° C./min, between 0.8 and 2° C./min or between 0.9 and 1.5° C./min. Preferably, the predefined rate is about 1° C./min.

The method may comprise contacting the phosphate-based glass with a therapeutic agent. The method may comprise contacting the phosphate-based glass with a solution comprising the therapeutic agent. The therapeutic agent may comprise an organic molecule. The organic molecule may be as defined above.

It may be appreciated that the statements provided below could be applied to the methods of either the third or fourth aspects.

The reaction solvent may be or comprise water. In some embodiments, the reaction solvent may be or comprise an alcohol. The alcohol may be as defined above.

The first and second solutions may be combined at a specific volumetric ratio. The volumetric ratio of the first and second solutions may be chosen based upon the concentrations of the components in the first and second solutions, and the desired ratios of the components in the reaction solution. The volumetric ratio of the first and second solutions may be between 1:20 and 20:1, between 1:10 and 10:1, between 1:5 and 5:1, between 1:3 and 3:1, between 1:2 and 2:1, between 1:1.5 and 1.5:1 or between 1:1.25 and 1.25:1. In some embodiments, the volumetric ratio of the first and second solutions may be about 1:1.

Providing the first solution may comprise dissolving the phosphate, and preferably the polyphosphate, in the first solvent.

The first solvent may be or comprise water.

The phosphate may be a polyphosphate. The polyphosphate may have a molecular weight of at least 300 g/mol, more preferably at least 500 g/mol at least 750 g/mol or at least 1,000 g/mol, and most preferably at least 1,500 g/mol, at least 2,000 g/mol or at least 2,500 g/mol. The phosphate may be provided with a counter ion. The counter ion may be sodium.

The reaction solution may comprise the phosphate at a concentration of between 0.001 and 15 M, between 0.01 and 12.5 M, between 0.1 and 10 M, between 0.25 and 7.5 M, between 0.5 and 5 M, between 1 and 3 M, between 1.5 and 2.5 M or between 1.75 and 2.25 M.

The concentration of the phosphate in the first solution may be selected depending upon the desired concentration of the phosphate in the reaction solution. The first solution may comprise the phosphate at a concentration of between 0.001 and 30 M, between 0.01 and 25 M, between 0.1 and 20 M, between 0.5 and 15 M, between 1 and 10 M, between 2 and 7.5 M, between 3 and 5 M or between 3.5 and 4.5 M.

In embodiments where the phosphate is a polyphosphate, the concentration may be understood to be the concentration of the phosphorous atoms from the polyphosphate in the first solution.

The surfactant may be a copolymer and/or a quaternary ammonium surfactant.

In some embodiments, the surfactant is a copolymer. The copolymer may be a triblock copolymer, and most preferably a symmetric triblock copolymer.

The copolymer is preferably an amphiphilic block copolymer. Accordingly, the copolymer may comprise one or more hydrophobic blocks and one or more hydrophilic blocks. Preferably, the amphiphilic block copolymer comprises a hydrophobic core and hydrophilic blocks comprising hydrophilic end groups.

The or each hydrophobic block may comprise poly(propylene oxide) (PPO). The or each hydrophilic block may comprise poly(ethylene oxide) (PEO). Accordingly, the copolymer may comprise poly(ethylene oxide)-block-poly(propylene oxide)-block-poly(ethylene oxide) (PEO-PPO-PEO). It may be appreciated that this copolymer can be referred to as Pluronic® block-copolymer. Suitable PluronicR block-copolymers are described in Barry et al. (“Pluronic® block-copolymers in medicine: from chemical and biological versatility to rationalisation and clinical advances”, Polym. Chem., 2014, 5, 3219), incorporated herein by reference. Accordingly, the copolymer may be referred to as a Poluronic® P-123, Pluronic® F-127, Pluronic® L61, Pluronic® L121, Pluronic® F68, Pluronic® F87 or Pluronic® P105.

The copolymer may have an average molecular weight between 500 and 50,000 g/mol or between 1,000 and 25,000 g/mol, more preferably between 2,000 and 20,000 g/mol or between 3,000 and 15,000 g/mol. In some embodiments, the copolymer may have an average molecular weight between 4,000 and 10,000 g/mol, between 4,500 and 9,000 g/mol, between 5,000 and 7,500 g/mol or between 5,500 and 6,000 g/mol. It is noted that P-123 has an average molecular weight of about 5,800 g/mol. In other embodiments, the copolymer may have an average molecular weight between 5,000 and 25,000 g/mol, between 8,000 and 20,000 g/mol, between 10,000 and 15,000 g/mol or between 12,000 and 13,000 g/mol. It is noted that F-127 has an average molecular weight of about 12,600 g/mol.

The or each hydrophobic block may have an average molecular weight between 500 and 20,000 g/mol, between 1,000 and 15,000 g/mol, between 1,500 and 10,000 g/mol or between 2,000 and 7,500 g/mol, more preferably between 2,500 and 5,000 g/mol or between 3,000 and 4,500 g/mol. In some embodiments, the or each hydrophobic block may have an average molecular weight between 3,250 and 4,750 g/mol, between 3,500 and 4,500 g/mol, between 3,750 and 4,250 g/mol or between 4,000 and 4,100 g/mol. It is noted that the hydrophobic core of P-123 has an average molecular weight of about 4,065 g/mol. In other embodiments, the or each hydrophobic block may have an average molecular weight between 2,500 and 4,000 g/mol, between 2,750 and 3,750 g/mol, between 3,000 and 3,500 g/mol or between 3,200 and 3,300 g/mol. It is noted that the hydrophobic core of F-127 has an average molecular weight of about 3,250 g/mol.

The or each hydrophilic block may have an average molecular weight between 100 and 20,000 g/mol, between 200 and 15,000 g/mol, between 400 and 10,000 g/mol or between 500 and 7,500 g/mol, more preferably between 600 and 6,000 g/mol or between 800 and 5,000 g/mol. In some embodiments, the or each hydrophilic block may have an average molecular weight between 200 and 4,000 g/mol, between 400 and 3,000 g/mol, between 600 and 2,000 g/mol, between 700 and 1,500 g/mol, between 800 and 1,000 g/mol or between 850 and 900 g/mol. It is noted that the hydrophilic end groups of P-123 each have an average molecular weight of about 880 g/mol. In other embodiments, hydrophobic block may have an average molecular weight between 1,000 and 10,000 g/mol, between 2,000 and 8,000 g/mol, between 3,000 and 6,000 g/mol, between 4,000 and 5,000 g/mol, between 4,250 and 4,750 g/mol or between 4,400 and 4,500 g/mol. It is noted that the hydrophilic end groups of F-127 each have an average molecular weight of about 4,450 g/mol.

All references to average molecular weight, unless otherwise specified, may be understood to refer to number average molecular weight (Mn).

In alternative embodiments, the surfactant is a quaternary ammonium surfactant. The quaternary ammonium surfactant may comprise a cation of formula (I):

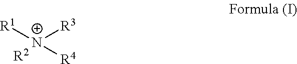

wherein, R¹ to R⁴ are independently an optionally substituted C₁₋₅₀ alkyl, an optionally substituted C₂₋₅₀ alkenyl or an optionally substituted C₂₋₅₀ alkynyl.

The alkyl, alkenyl and/or alkynyl groups may be straight or branched chain alkyl, alkenyl and/or alkynyl groups. The alkyl, alkenyl and/or alkynyl groups may be unsubstituted or substituted with one or more of halide or OH. Preferably, the alkyl, alkenyl and/or alkynyl groups are unsubstituted.

R¹ to R⁴ may independently be an optionally substituted C₁₋₄₀ alkyl, an optionally substituted C₂₋₄₀ alkenyl or an optionally substituted C₂₋₄₀ alkynyl. Preferably, R¹ to R⁴ are independently an optionally substituted C₁₋₃₀ alkyl, an optionally substituted C₂₋₃₀ alkenyl or an optionally substituted C₂₋₃₀ alkynyl. More preferably, R¹ to R⁴ are independently an optionally substituted C₁₋₂₀ alkyl, an optionally substituted C₂₋₂₀ alkenyl or an optionally substituted C₂₋₂₀ alkynyl.

R¹ to R³ may independently be an optionally substituted C₁₋₁₅ alkyl, an optionally substituted C₂₋₁₅ alkenyl or an optionally substituted C₂₋₁₅ alkynyl. More preferably, R¹ to R³ are independently an optionally substituted C₁₋₁₀ alkyl, an optionally substituted C₂₋₁₀ alkenyl or an optionally substituted C₂₋₁₀ alkynyl. Even preferably, R¹ to R³ are independently an optionally substituted C₁₋₅ alkyl, an optionally substituted C₂₋₅ alkenyl or an optionally substituted C₂₋₅ alkynyl. Accordingly, R¹ to R³ may independently be methyl, ethyl, propyl, butyl or pentyl. In some embodiments, R¹ to R³ are each methyl.

R⁴ may be an optionally substituted C₅₋₂₀ alkyl, an optionally substituted C₅₋₂₀ alkenyl or an optionally substituted C₅₋₂₀ alkynyl. More preferably, R⁴ may be an optionally substituted C₁₀₋₂₀ alkyl, an optionally substituted C₁₀₋₂₀ alkenyl or an optionally substituted C₁₀₋₂₀ alkynyl. Even more preferably, R⁴ may be an optionally substituted C₁₃₋₁₈ alkyl, an optionally substituted C₁₃₋₁₈ alkenyl or an optionally substituted C₁₃₋₁₈ alkynyl. In some embodiments, R⁴ is hexadecanyl.

The quaternary ammonium surfactant may also comprise an anion. The anion may be a nitrate, an acetate, a sulphate or a halide. The halide may be a fluoride, chloride, bromide or iodide. In some embodiments, the halide may be bromide.

Accordingly, the surfactant may be cetrimonium bromide (CTAB).

The reaction solution preferably comprises the surfactant in an amount between 0.0001 and 30% (w/v), more preferably in an amount between 0.001 and 20% (w/v), between 0.0025 and 10% (w/v) or between 0.005 and 7.5% (w/v), and most preferably in an amount between 0.0075 and 5% (w/v), between 0.01 and 2.5% (w/v), between 0.025 and 1% (w/v) or between 0.05 and 0.5% (w/v).

The concentration of the surfactant in the second solution may be selected depending upon the desired concentration of the surfactant in the reaction solution. In some embodiments, the first solution preferably comprises the surfactant in an amount between 0.0001 and 60% (w/v), more preferably in an amount between 0.005 and 50% (w/v), between 0.001 and 20% (w/v) or between 0.05 and 15% (w/v), and most preferably in an amount between 0.01 and 10% (w/v), between 0.025 and 7.5% (w/v) between 0.05 and 5% (w/v), between 0.075 and 2.5% (w/v) or between 0.1 and 1% (w/v).

The method may comprise:
providing a third solution comprising the therapeutic agent, preferably the therapeutic metal or therapeutic metal cation, and a third solvent; and
combining the first solution, the second solution and the third solution to provide the reaction solution.

Preferably, the method comprises:
combining the first and second solutions to provide a combined solution;
and subsequently combining the combined solution and the third solution to provide the reaction solution.

Accordingly, the reaction solvent may comprise a mixture of the first, second and third solvents.

The third solvent may be or comprise water.

The concentration of the therapeutic metal or therapeutic metal cation in the reaction solution may be between 0.005 and 1 M, between 0.01 and 0.75 M, between 0.02 and 0.5 M, between 0.04 and 0.3 M, between 0.06 and 0.2 M, between 0.08 and 0.15 M or between 0.10 and 0.12 M.

The concentration of the therapeutic metal or therapeutic metal cation in the third solution may be selected depending upon the desired concentration of the therapeutic metal or therapeutic metal cation in the reaction solution. The third solution may comprise the therapeutic metal or therapeutic metal cation at a concentration of between 0.001 and 25 M, between 0.01 and 20 M, between 0.05 and 15 M, between 0.1 and 10 M, between 0.25 and 7.5 M, between 0.5 and 5 M, between 1 and 3M or between 1.5 and 2.5 M.

The third solution and the combined solution may be combined at a specific volumetric ratio. The volumetric ratio of the third solution and the combined solution may be chosen based upon the concentrations of the components in the third solution and the combined solution, and the desired ratios of the components in the reaction solution. The volumetric ratio of the third solution and the combined solution may be between 2:1 and 1:50, between 1:1 and 1:40, between 1:5 and 1:30, between 1:10 and 1:25, between 1:12.5 and 1:20 or between 1:15 and 1:18.

In accordance with a fifth aspect, there is provided a fibre comprising or consisting of a porous phosphate-based glass obtained or obtainable using the method of the third aspect.

In accordance with a sixth aspect, there is provided a phosphate-based glass comprising a combination of calcium oxide and magnesium oxide obtained or obtainable using the method of the fourth aspect.

In accordance with a seventh aspect, there is provided the fibre of the first or fifth aspects or the phosphate-based glass of the second or sixth aspects for use in therapy.

In accordance with an eighth aspect, there is provided the fibre of the first or fifth aspects or the phosphate-based glass of the second or sixth aspects for use in (a) treating an infection, (b) drug delivery, (c) bone regeneration and/or (d) wound healing.

In accordance with a ninth aspect, there is provided a method of (a) treating an infection, (b) drug delivery, (c) bone regeneration and/or (d) wound healing, the method comprising administering the fibre of the first or fifth aspects or the phosphate-based glass of the second or sixth aspects to a patient in need thereof.

All features described herein (including any accompanying claims, abstract and drawings), and/or all of the steps of any method or process so disclosed, may be combined with any of the above aspects in any combination, except combinations where at least some of such features and/or steps are mutually exclusive.

### EXAMPLES

### Example 1—Preliminary Surfactant Solubility Experiment

A series of experiments were initially conducted to investigate the best method for addition of surfactant:
1) P123 and F127 surfactant at 0.1, 1, and 5% w/v (weight/volume) were added to de-ionised water and 20 and 70% (v/v) ethanol solutions.
2) P123 and F127 were added to pre-prepared 4M NaPP solution.
3) NaPP powder was added to various concentrations of F127 and P123 surfactant while stirring.

Method 2 and 3, were not successful since the surfactant was not soluble in solution and a white layer at the bottom of the solution was formed, which did not remove with further stirring. Method 1 was successful and taken forward since the surfactant was soluble in de-ionised water and at various ethanol concentrations.

### Example 2—Synthesis of Porous Glass and Fibres in the System P₂O₅—CaO—Na₂O Using Pluronic F127 as a Surfactant

A 4M polyphosphate solution was first prepared by mixing sodium polyphosphate ((NaPP3)ₙ; Merck, 99.0%, 101.962 g) with de-ionised water (DW, 250 ml). Initially, Pluronic F127 surfactant, which is a block copolymers of poly(ethylene oxide)-poly(propylene oxide)-poly(ethylene oxide) (PEO-PPO-PEO) was used to produce F127 solutions at concentrations of 0.1% w/v (0.0794 mM), 1% w/v (0.7937 mM) and 5% w/v (3.9683 mM). This was achieved by adding 0.2, 2 and 10 g of F127 surfactant to 200 ml of DW, 10, 20 and 70% (v/v) ethanol solutions, prepared with graded ethanol, thus producing a total of 12 solutions (Table 1). These solutions were stirred for ˜1 h or until fully dissolved, producing a clear solution. These concentration values were chosen since the critical micelle concentration (CMC) for P123 and F127 is 0.313 mM and 0.431 mM, respectively (Ding et al., 2003; He and Alexandridis, 2017).

Subsequently, 25 ml of each the 12 F127 solutions were added to separate containers and 11.8075 g of calcium nitrate tetrahydrate solution (Ca(NO₃)₂·4H₂O; Acros, 99.0%) was added and stirred for a further 30 min to produce 2M calcium nitrate solutions. 20 ml of the F127/Ca(NO₃)₂ solution was added dropwise to 20 ml of the prepared NaPP solution using a syringe pump (0.3 ml min⁻¹) whilst vigorously stirring. It will be appreciated that the concentration of ethanol in these solutions was 0, 5, 10 and 35% (v/v) and the amount of the F127 surfactant was undoped or 0.05, 0.5 and 2.5% (w/v). However, for the avoidance of doubt, the final products will be referred to in relation to the amount of ethanol and/or surfactant in the initial F127 solutions, i.e. 0, 10, 20 or 70% (v/v) ethanol and 0.1, 1 or 5% (w/v) F127.

After addition, the mixture was stirred for a further hour, where a phase separation occurred and produced a coacervate glass layer at the bottom. The coacervate was allowed to settle for a further 24 h before removing the supernatant. Each coacervate was split in half, one half to produce fibres and the other to vacuum dry to produce powders.

In order to produce porous powders, the samples were vacuum dried at room temperature for 48 h or until dry, before calcination at 300° C. (ramp speed 1° C./min to 300° C., dwell time: 30 min) to produce porous powders.

To produce porous fibres, electrospinning was performed on the other half of coacervate at room temperature using a stainless-steel capillary (gauge 18 nozzle), syringe pump, and high voltage source (Spraybase system, Kildare, Ireland). The distance between the capillary tip and the collector was set at 15 cm, the flow rate was set at 2.0 ml·h⁻¹ and a voltage of 17.5 kV was applied between the nozzle and a metal screen collector, where PGF were deposited. The fibres were then calcinated using the same method as described above. Note, all samples were attempted however only undoped (F127 free) and 0% ethanol samples produced fibres, since the viscosity of the coacervate increased with ethanol and electrospinning was not possible.

The relative molar amounts of P2O5, CaO and Na2O in these glasses were:

$${P}_{2} {O}_{5} = 0.49 ;$$

$$CaO = 0.37 ; and$$

$${Na}_{2} O = 0.14 .$$

TABLE 1
Synthesised samples of porous phosphate-
based glasses containing Pluronic F127

| F127 surfactant phosphate-based glass (PBG) | Powder | Fibre |
|---|---|---|
| Undoped coacervate glass, 0% (v/v) ethanol | ✓ | ✓ |
| 0.1, 1 and 5 w/v % F127 PBG, 0% (v/v) ethanol | ✓ | ✓ |
| 0.1, 1 and 5 w/v % F127 PBG, 10% (v/v) ethanol | ✓ | X |
| 0.1, 1 and 5 w/v % F127 PBG, 20% (v/v) ethanol | ✓ | X |
| 0.1, 1 and 5 w/v % F127 PBG, 70% (v/v) ethanol | ✓ | X |

### Results

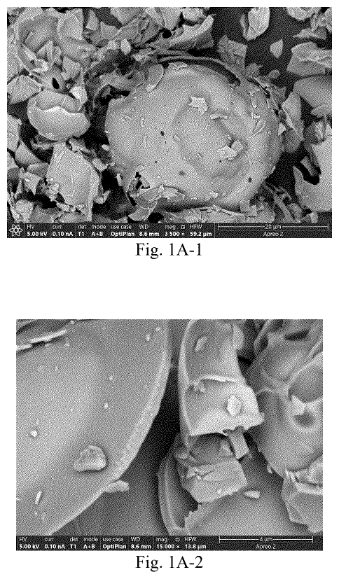

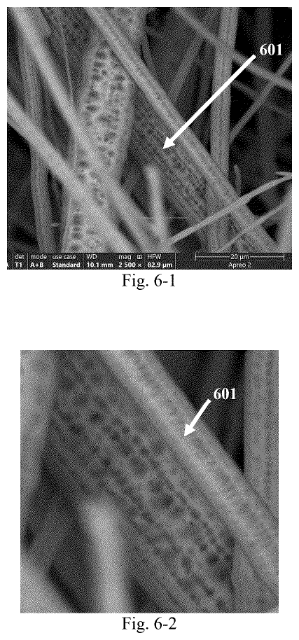

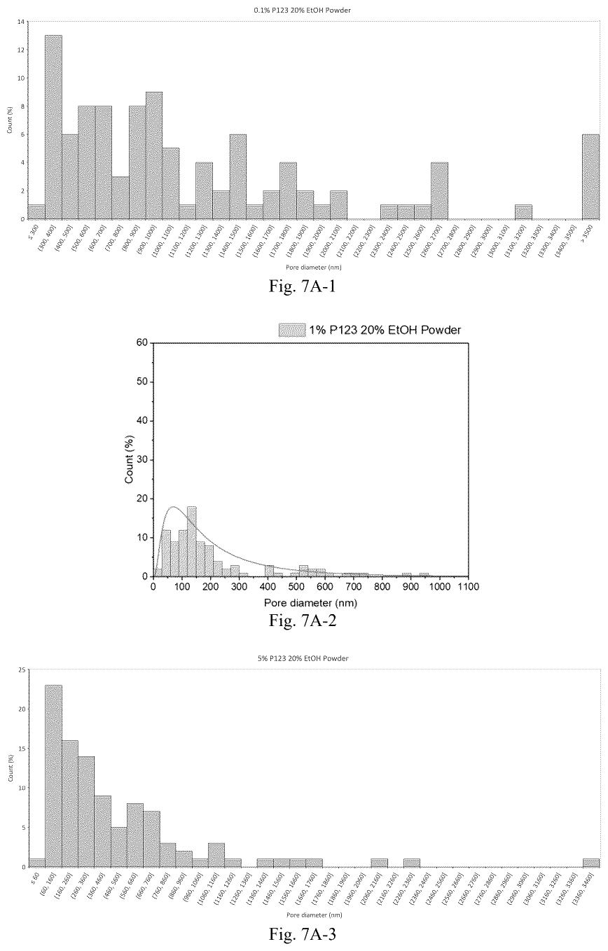

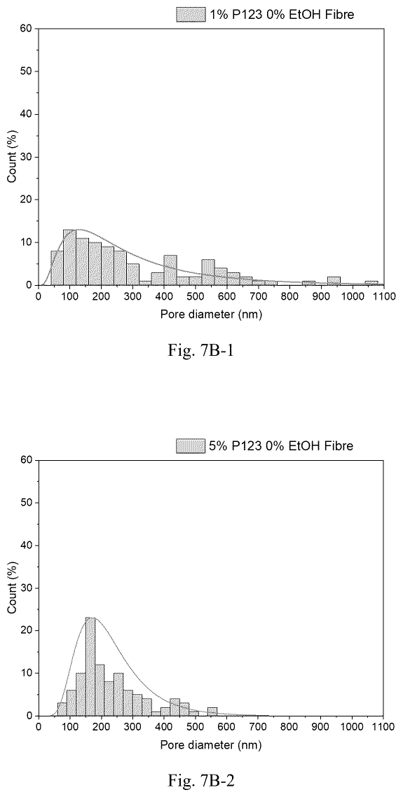

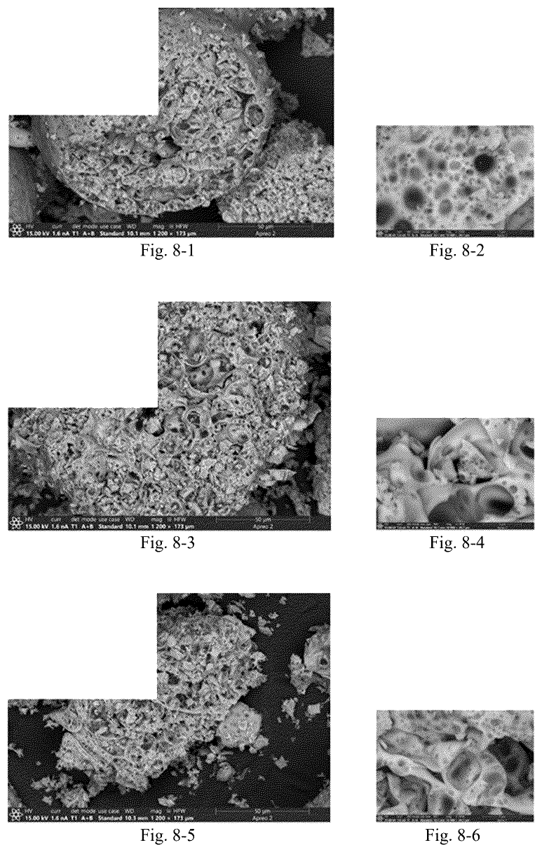

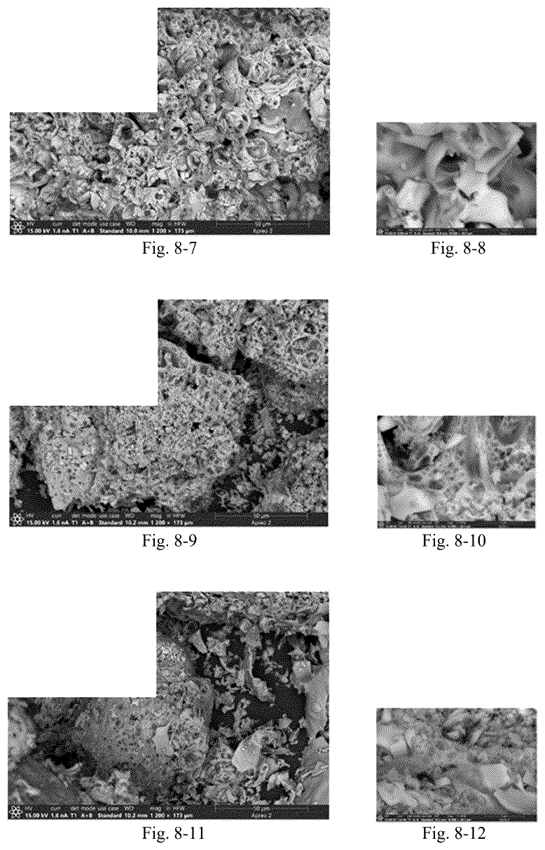

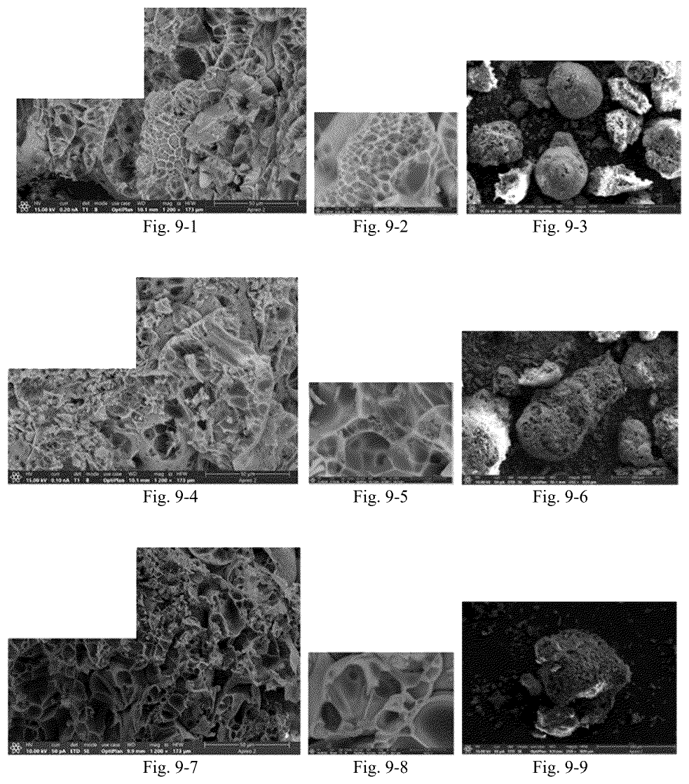

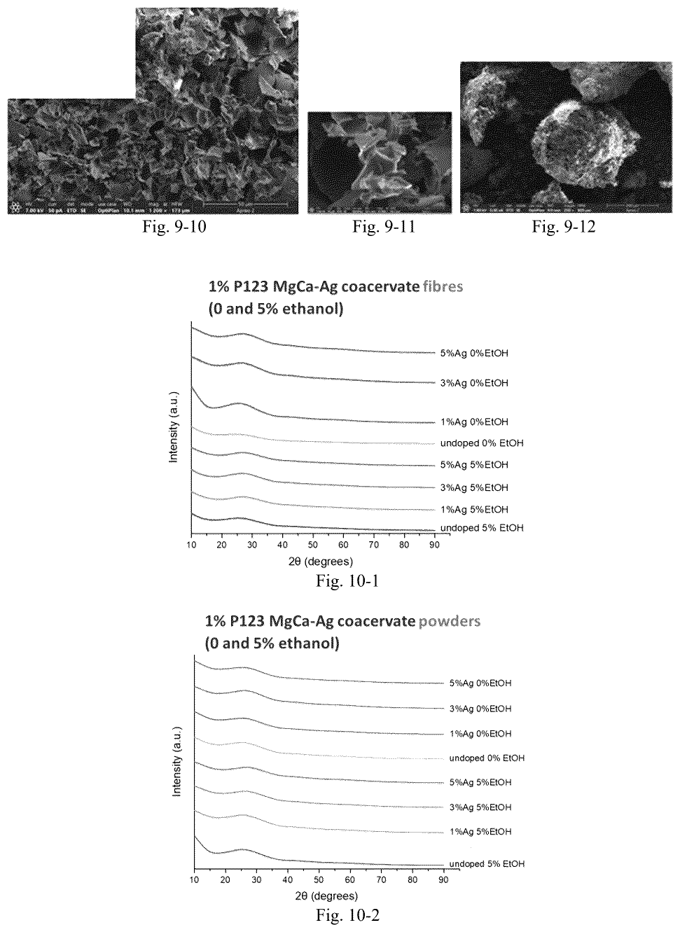

Samples were analysed using scanning electron microscopy (SEM), as shown in FIGS. 2A-1 to 2A-22, and 2B-1 to 2B-6 and the images were then analysed using ImageJ software to provide the graphs shown in FIGS. 3A-1 to 3A-3, 3B-1 to 3B-3, 3C-1 to 3C-3, and 3D-1 to 3D-3.

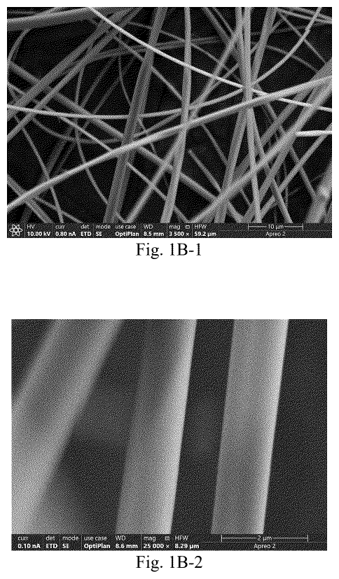

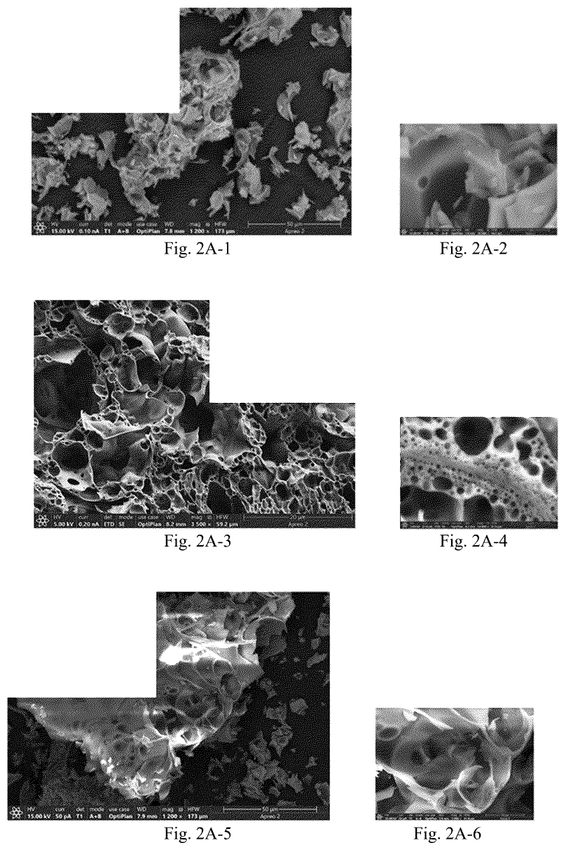

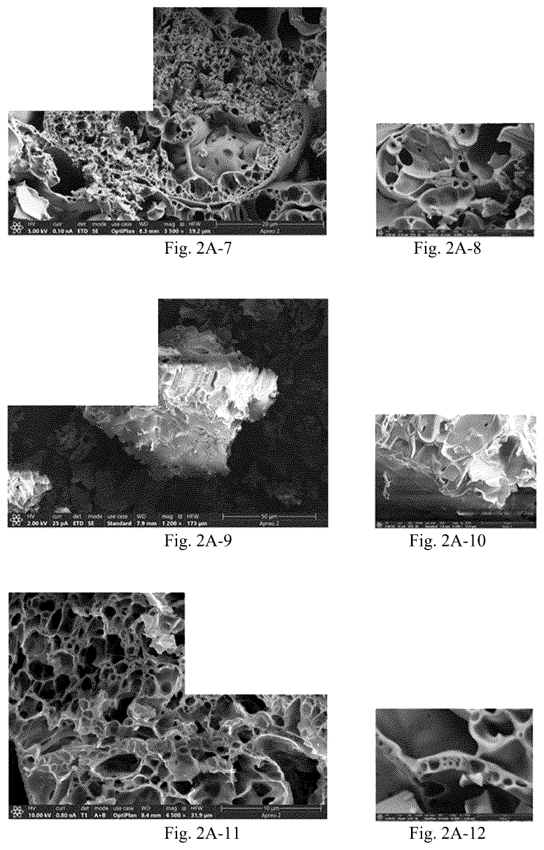

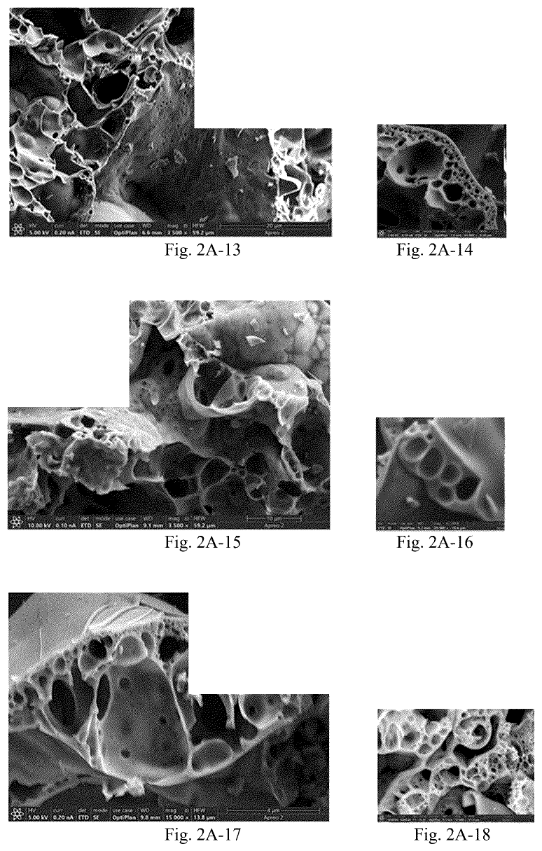

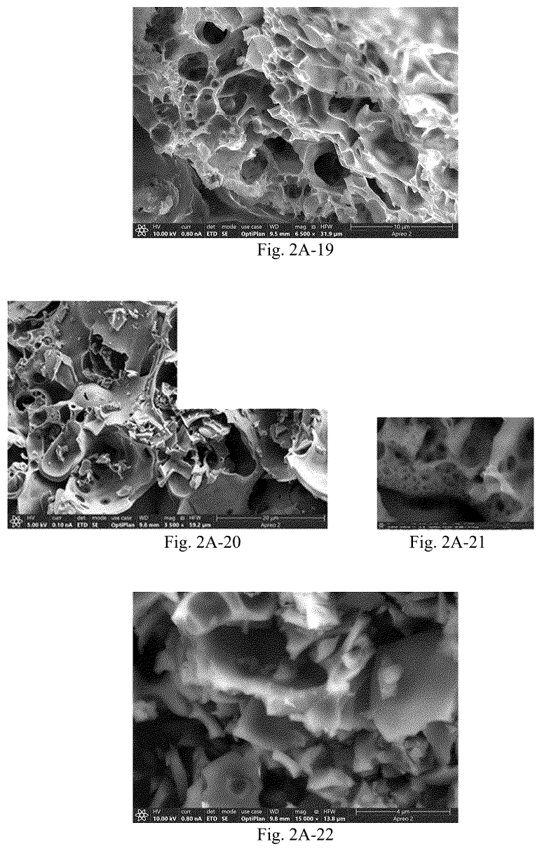

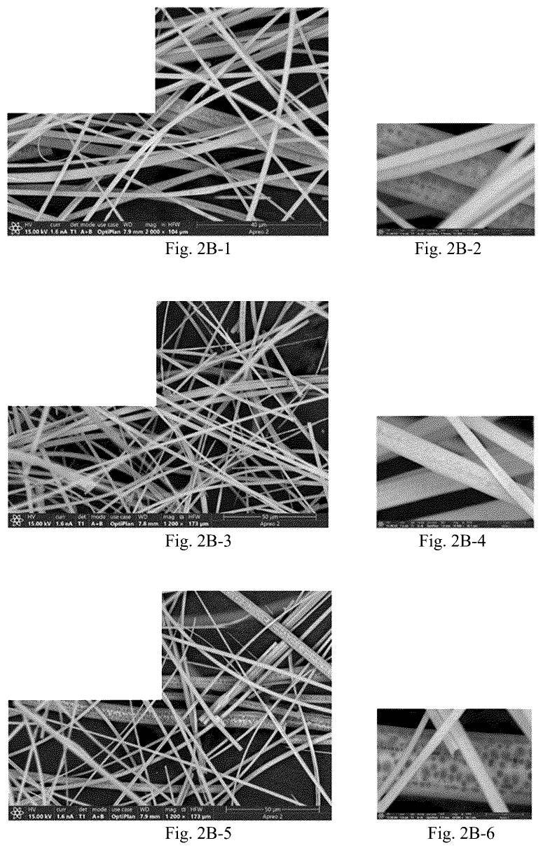

The coacervate fibre and powder controls which did not contain surfactant, did not show any porosity after calcination, as seen in FIGS. 1A-1, 1A-2, 1B-1, and 1B-2.

As shown in FIGS. 2A-1 to 2A-22, 3A-1 to 3A-3, and 2B-1 to 2B-6, coacervate powders F127 containing 10% and 20% ethanol demonstrated the greatest hierarchical porosity, including nanopores in the range of 50-100 nm.

Fibres were only successfully produced with the 0% ethanol coacervate since the viscosity was not as great and would easily flow for electrospinning. Some porosity can be seen along the fibres of the 5% w/v F127 coacervate, see FIGS. 2B-5, 2B-6, and 3D-3, something not previously seen. This could be a great advantage in terms of release of therapeutic molecules from porous fibres.

Imaging the above coacervate F127 powder samples at a lower magnification (FIGS. 4-1 and 4-2), it was clearly observed that porous microspheres were being produced.

The median pore and median fibre diameters (D(50)) were calculated using the ImageJ software. For each measurement, 100 pore diameters or 100 fibre diameters were measured. The results are provided in tables 5 and 6 below.

TABLE 2
Median pore diameter for the samples of porous phosphate-
based glasses containing Pluronic F127

| | Powders | | | | Fibres |
|---|---|---|---|---|---|
| | 0% | 10% | 20% | 70% | 0% |
| | (v/v) | (v/v) | (v/v) | (v/v) | (v/v) |
| | EtOH | EtOH | EtOH | EtOH | EtOH |
| 0.1% w/v | No | 60.5 | 53 | 904 | 465 |
| F127 | porosity | nm | nm | nm | nm |
| 1% w/v | No | 357 | 114 | 255 | 178 |
| F127 | porosity | nm | nm | nm | nm |
| 5% w/v | No | 339 | 222 | 699 | 391 |
| F127 | porosity | nm | nm | nm | nm |

TABLE 3
Average fibre diameter for the porous phosphate-
based glass fibres containing Pluronic F127

| | Average fibre diameter |
|---|---|
| 0.1% w/v F127 | 1.54 ± 0.86 μm |
| 1% w/v F127 | 1.63 ± 0.83 μm |
| 5% w/v F127 | 1.78 ± 1.07 μm |

### Example 3—Synthesis of Porous Glass and Fibres in the System P₂O₅—CaO—Na₂O Using Pluronic P123 as a Surfactant

Using the method above to produce P₂O₅—CaO—Na₂O+surfactant glasses, coacervates were produced again using the same method. However, the F127 surfactant was replaced with P123 using 0% ethanol and 20% ethanol solutions. Fibres and powders were successfully produced only with the 0% ethanol (Table 4).

All samples prepared (powders and fibres) are listed in Table 1. Starting from the same coacervate gel, only some fibres could be produced due to the viscosity.

TABLE 4
Synthesised samples of porous phosphate-
based glasses containing Pluronic P123

| P123 surfactant PBG | Powder | Fibre |
|---|---|---|
| 0.1, 1 and 5 w/v % P123 PBG, 0% ethanol | ✓ | ✓ |
| 0.1, 1 and 5 w/v % P123 PBG, 20% ethanol | ✓ | X |

### Results

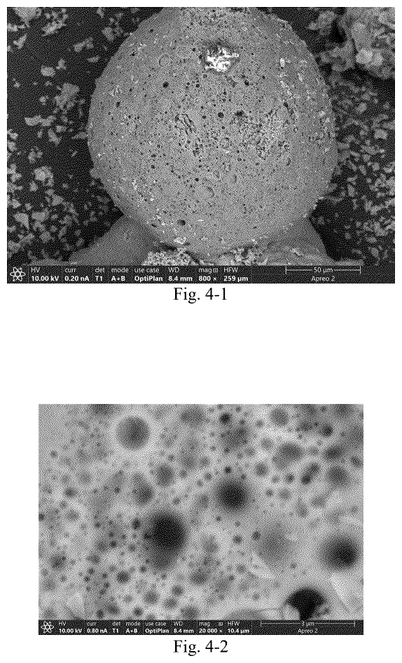

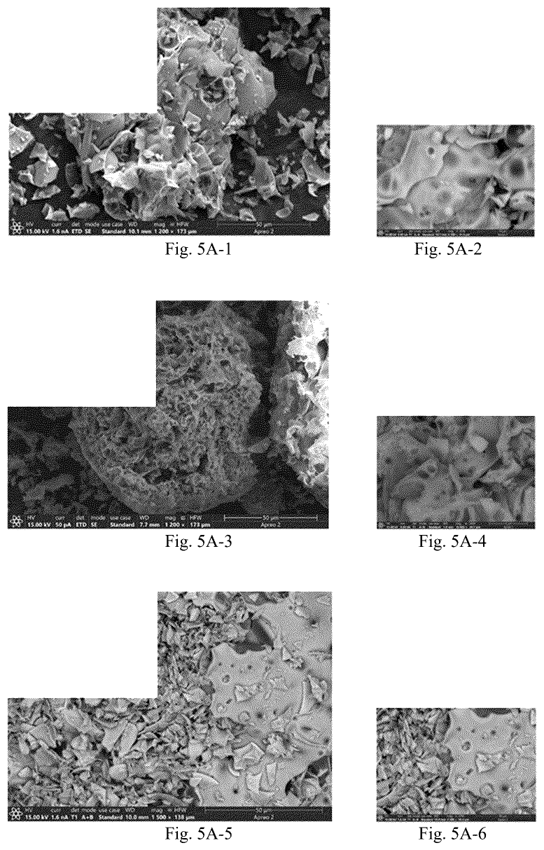

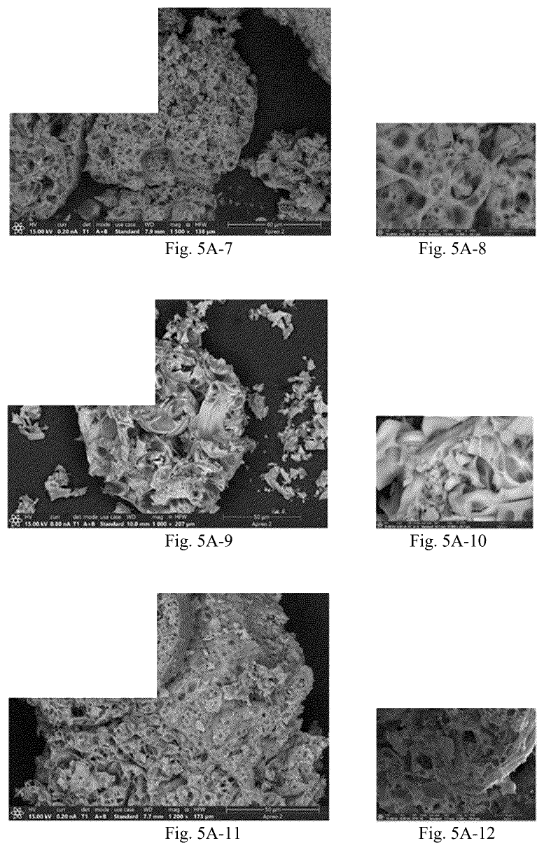

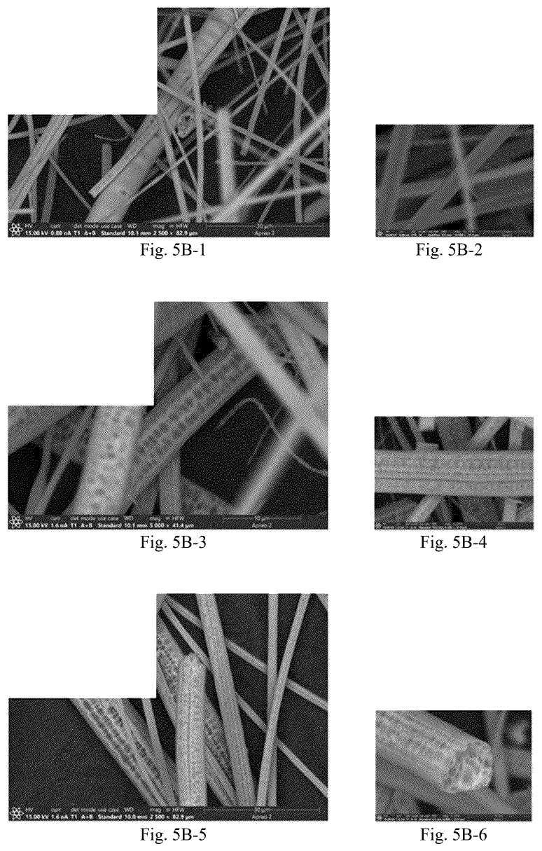

The use of ethanol at 20% (v/v) had an influence on the amount of porosity in the glass powders. In FIG. 5A 0% (v/v) ethanol demonstrates far less porosity in comparison to the 20% (v/v) ethanol during preparation of the P123 PBG.

As with example 2, fibres were only successfully produced with the 0% ethanol coacervate. Aligned nanopores 601 can be seen along the fibres of both the 1 and 5% w/v P123 coacervate, see FIGS. 5B-3 to 5B-6, 6-1, and 6-2.

The average diameter of the pores was analysed using ImageJ software, as shown in FIGS. 7A-1 to 7A-3 and 7B-1 to 7B-2, and the median pore and median fibre diameters were calculated. The results are provided in tables 5 and 6 below.

TABLE 5
Median pore diameter for the samples of porous phosphate-
based glasses containing Pluronic P123

| | Powder | Fibres |
|---|---|---|
| | (20% (v/v) EtOH) | (0% (v/v) EtOH) |
| 0.1% w/v P123 | 924 nm | No porosity |
| 1% w/v P123 | 145 nm | 233 nm |
| 5% w/v P123 | 311 nm | 196 nm |

TABLE 6
Average fibre diameter for the porous phosphate-
based glass fibres containing Pluronic P123

| | Average fibre diameter |
|---|---|
| 0.1% w/v P123 | 1.54 ± 0.86 μm |
| 1% w/v P123 | 1.63 ± 0.83 μm |
| 5% w/v P123 | 1.78 ± 1.07 μm |

### Example 4—Investigating the Effect of Calcination Ramp Speed on F127 PBG Powder Porosity

Porous coacervates synthesised as described above were further investigated to understand the effect of ramp speed on their porosity. Coacervates produced using 20% ethanol were chosen since they demonstrated the greatest porosity. Again, a concentration of 0.1, 1 and 5 w/v % was used to prepare the coacervates and a 0.5 and 5° C. ramp speed was introduced (Table 7). Dwell time was 30 minutes.

TABLE 7
Synthesised porous phosphate-based glass
powders containing F127 surfactants.

| F127 surfactant PBG | Ramp speed |
|---|---|
| 0.1, 1 and 5 w/v % F127 PBG, 20% | 0.5° C./min |
| ethanol | |
| 0.1, 1 and 5 w/v % F127 PBG, 20% | 1° C./min |
| ethanol | |
| 0.1, 1 and 5 w/v % F127 PBG, 20% | 5° C./min |
| ethanol | |

### Results

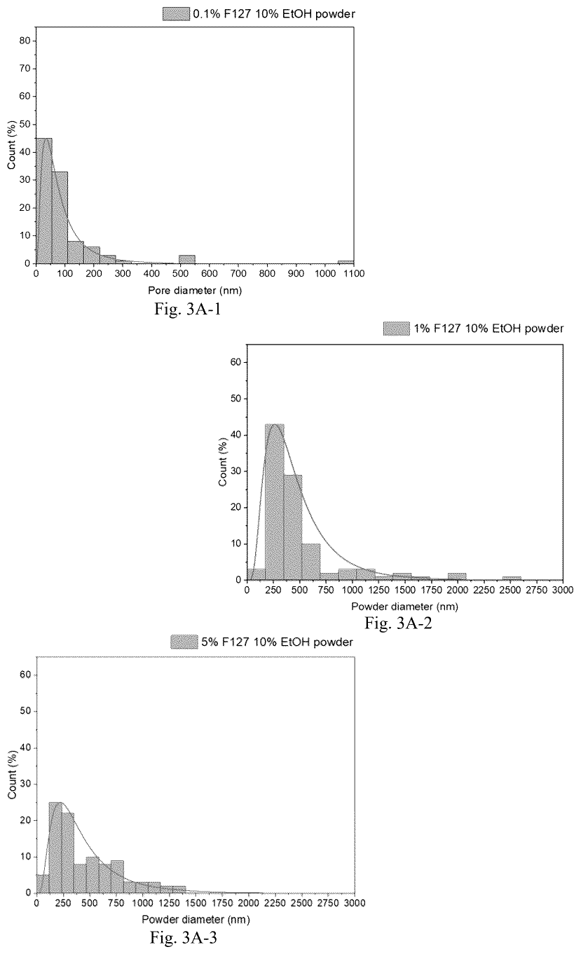

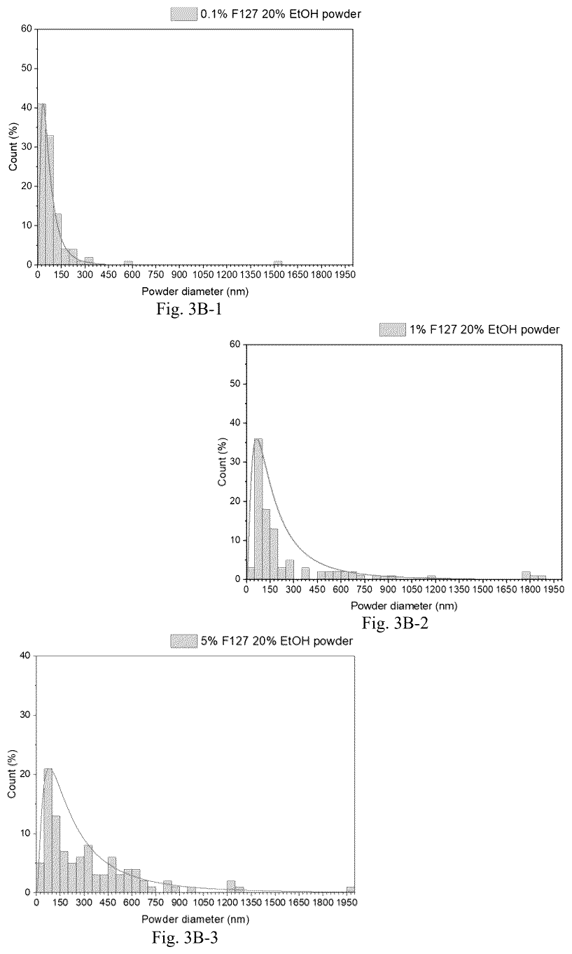

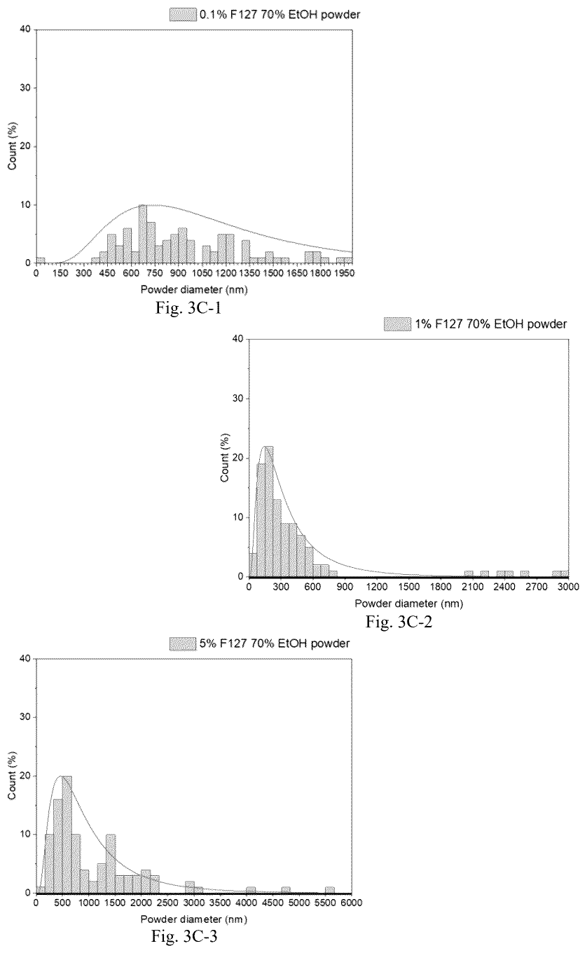

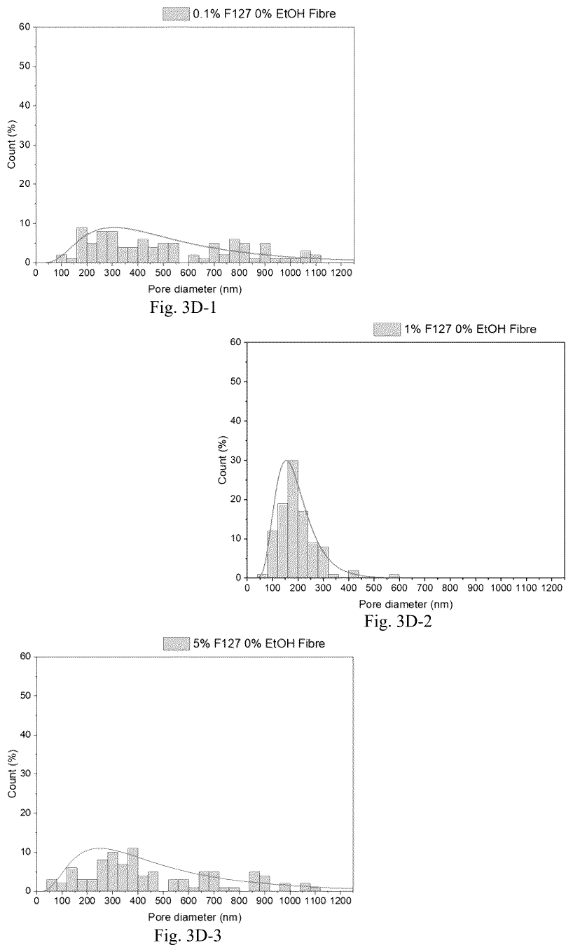

As shown in FIGS. 8-1 to 8-11 and the 20% (v/v) ethanol sample in FIGS. 5A-3, 5A-4, 5A-7, 5A-8, 5A-11, and 5A-12, the synthesis process involving the ramp rate during calcination had a great impact on the porosity of the coacervates. The ramp rate of 1° C./min (FIGS. 5A-3, 5A-4, 5A-7, 5A-8, 5A-11, and 5A-12) demonstrated the greatest porosity, in comparison to the 0.5° C./min and 5° C./min ramp rates.

### Example 5—Synthesis of Porous Glass and Fibres in the System P₂O₅—CaO—MgO—Na₂O with the Addition of Ag⁺ Using Pluronic P123 as a Surfactant

1% (w/v) P123 showed the best glass/fibre porosity, including both large and small pores vs F127 and various concentrations of P123. Therefore, P123 at 1% w/v was taken forward to produce MgCa—Ag coacervate powders and fibres.

Using the method above to produce P₂O₅—CaO—Na₂O+surfactant glasses, coacervates were produced again using a similar method, however magnesium nitrate hexahydrate (Mg(NO₃)₂·6H₂O; Sigma Aldrich 99%) and silver nitrate (AgNO₃) were introduced to produce MgCa—Ag coacervates (P₂O₅—CaO—MgO—Na₂O with the addition of Ag⁺).

In detail, to produce the surfactant solution, 1 g of P123 surfactant was added to 100 ml of DW and to 100 ml of 5% ethanol, which was then stirred for 30 min. To produce a 1M Mg and 1M Ca solution, 23.615 g of calcium nitrate tetrahydrate (Ca(NO₃)₂·4H₂O; Acros, 99.0%) and 25.641 g of magnesium nitrate hexahydrate (Mg(NO₃)₂·6H₂O; Sigma Aldrich 99%) was added to the 100 ml P123 solutions, which was stirred again for a further 30 min. For each composition (Table 8) 20 ml of the P123/Ca(NO₃)₂/Mg(NO₃)₂ solution was added dropwise to 20 ml of the prepared NaPP solution using a syringe pump (0.3 ml min−1) whilst vigorously stirring. After adding this, the coacervates were doped with a preprepared 2M silver nitrate (AgNO₃, 16.987 g in 50 ml DW) to obtain the desired concentration e.g. adding 1.2 ml, 3.6 ml or 6.0 ml of 2M silver nitrate to obtain 1, 3 and 5 mol % Ag and stirred for a further 1 h. Again, the coacervates was allowed to settle for a further 24 h before removing the supernatant. Each coacervate was split in half, one half to produce fibres and the other to vacuum dry to produce powders. To produce powders, the samples were vacuum dried at room temperature for 48 h or until dry, before calcination at 300° C. (ramp speed 1° C./min to 300° C., dwell time: 30 min) to produce porous powders. To produce fibres, electrospinning was conducted using the same parameters as described above, and the fibres were then calcinated as described above.

It is noted that fibres were successfully obtained for the 5% (v/v) ethanol solutions, indicating that it is possible to obtain powders when less than 10% (v/v) ethanol is used.

TABLE 8
Porous phosphate-based glass powders and fibres containing 1 w/v
% P123 surfactants synthesised using 0 and 5% (v/v) ethanol.

| 1% w/v P123 surfactant, 0% | 1% w/v P123 surfactant, 5% |
|---|---|
| ethanol | ethanol |
| undoped fibre | undoped fibre |
| 1 mol % Ag fibre | 1 mol % Ag fibre |
| 3 mol % Ag fibre | 3 mol % Ag fibre |
| 5 mol % Ag fibre | 5 mol % Ag fibre |
| undoped powder | undoped powder |
| 1 mol % Ag powder | 1 mol % Ag powder |
| 3 mol % Ag powder | 3 mol % Ag powder |
| 5 mol % Ag powder | 5 mol % Ag powder |

TABLE 9
Relative molar compositions of undoped and doped
phosphate-based glass powders and fibres.

| Sample | P₂O₅ | CaO | MgO | Na₂O | Ag₂O |
|---|---|---|---|---|---|
| Undoped | 49.16 | 22.69 | 14.71 | 13.45 | — |
| 1% Ag | 51.03 | 25.89 | 11.26 | 10.51 | 1.31 |
| 3% Ag | 43.62 | 38.85 | 7.42 | 6.36 | 3.75 |
| 5% Ag | 48.84 | 22.10 | 12.82 | 10.61 | 5.64 |

### Results

With the addition of magnesium and silver ions to the PBG systems, it can be clearly seen (FIGS. 9-1 to 9-12 and Table 10) that the porosity is maintained in the structures. An increase in silver ions (Ag⁺) to 5% decreases the amount of nanopores within the system, although the larger micropores are present.

TABLE 10
Average pore size for the porous phosphate-
based glass powders containing 1 w/v % P123
surfactant synthesised using 5% (v/v) ethanol.

| Sample | D(50) | |
|---|---|---|
| Undoped | 253.9 | nm |
| 1% Ag | 358.5 | nm |
| 3% Ag | 1413.9 | nm |
| 5% Ag | 964.3 | nm |

As shown in FIGS. 10-1 and 10-2, all MgCa—Ag fibres and powders were found to be amorphous. This is something that was desired since amorphous phosphates can release ions in solution in a controlled way.

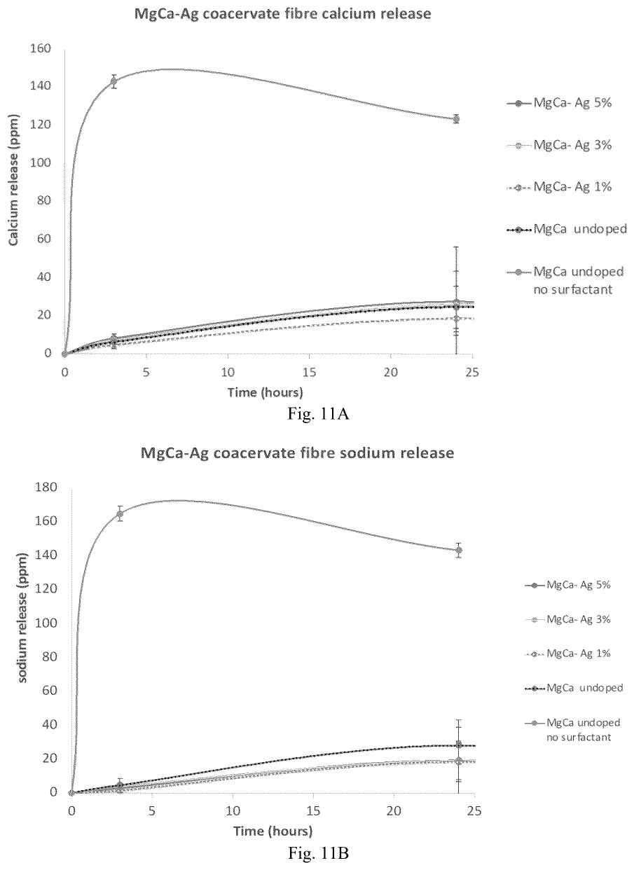

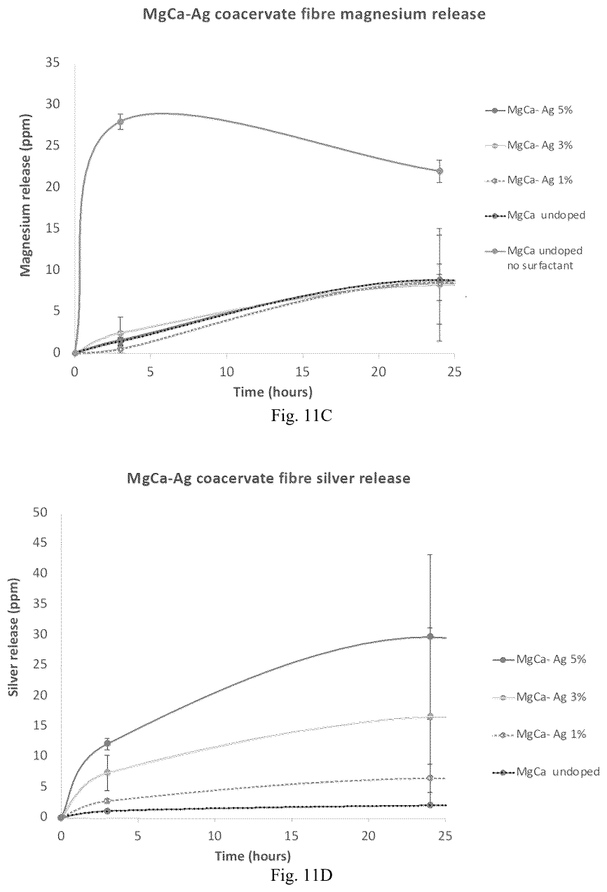

As shown in FIGS. 11A-D, porous fibres release is much slower than non-porous-avoiding the “burst” effect.

### Example 6—Assessing the Ability of the Porous Phosphate-Based Glass to Enhance Wound Closure

The inventors investigated the ability of porous phosphate-based glass produced in example 5 to enhance wound closure on human skin biopsies.

### Results

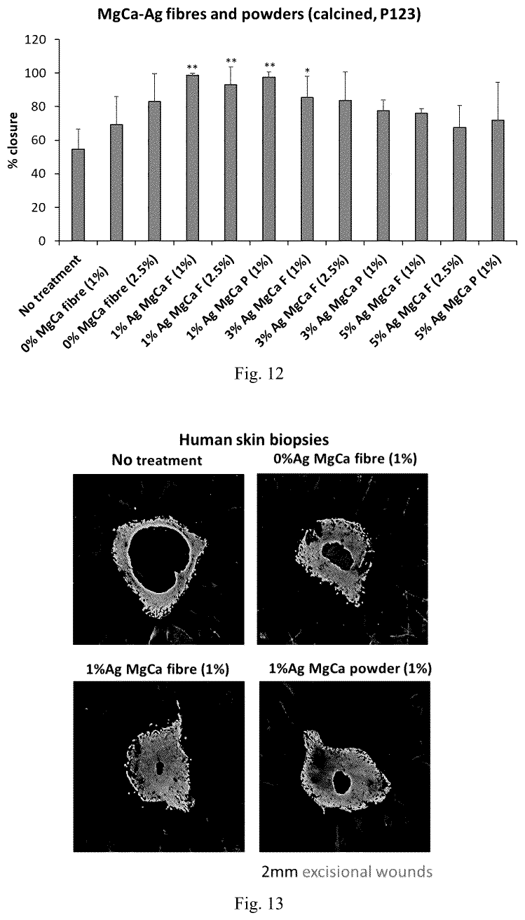

1% Ag (1% treatment in growth media) porous phosphate-based fibres (PBF) showed the greatest % wound closure, with fibres showing more promising results compared to powders. The wound closure for this treatment was close to 100% as shown in FIG. 12, thus demonstrating significant complete closure in comparison to the no treatment (growth media only) samples. In FIG. 13, it can be clearly seen on the confocal images of the wound, that the excisional wound has almost completed closed for the 1% Ag fibre sample. This is a significant result. The inventors note that they were unable to achieve such a great % wound closure with previous samples, including non-porous coacervates containing silver at different concentrations.

### Example 7—Comparing the Wound Healing Properties of the MgCa—Ag Compositions to the Ca—Ag Compositions

The inventors wished to ascertain the effect the addition of magnesium had on the compositions. Accordingly, the inventors prepared Ca—Ag samples using a method analogous to the one described in example 5, except instead of using a 1M Mg and 1M Ca solution, they instead used a 2M Ca solution.

### Results

The results are shown in FIGS. 14A and 14B. It will be noted that the 1% Ag MgCa fibres demonstrate the highest wound closure. It is noted that the addition of magnesium to the composition enhances the compositions ability to promote wound closure, particularly for the 1% Ag fibres.

### Example 8—Assessing the Antimicrobial Properties of the Porous Phosphate-Based Glass

The inventors investigated the ability of the porous phosphate-based glass produced in example 5 to inhibit bacteria. The inventors also assessed the cytotoxicity of the porous phosphate-based glasses produced in examples 5 and 7.

### Results

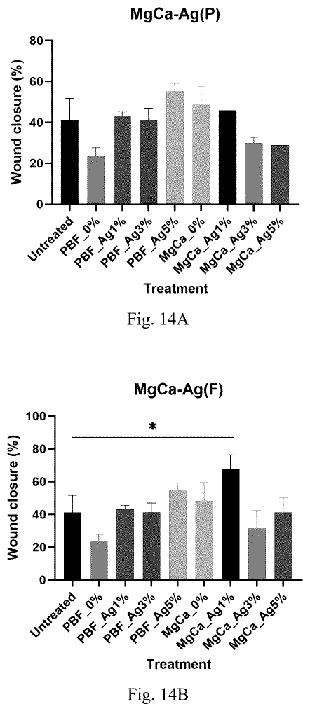

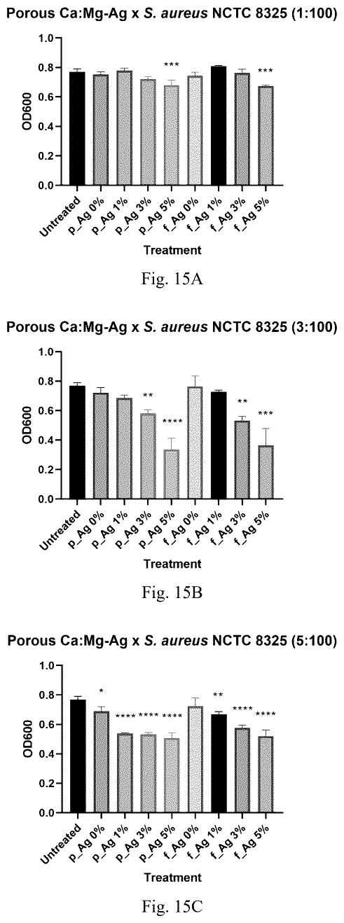

As shown in FIGS. 15A-15C, an antimicrobial effect against S. aureus is observed for 5% Ag the porous glass powders and fibres at all concentrations. Additionally, 3% Ag porous glass powders and fibres inhibited S. aureus when provided at a weight ratio of 3:100 or 5:100 glass to growth media, and 1% porous glass powders and fibres inhibited S. aureus when provided at a weight ratio of 5:100 glass to growth media.

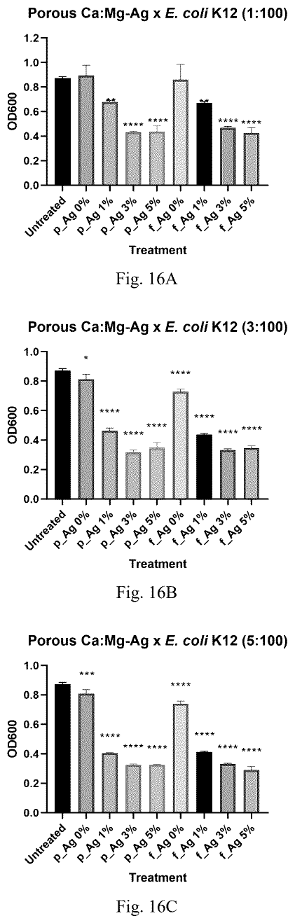

As shown in FIGS. 16A-16C, the antimicrobial effect was stronger against E. coli with the 1% Ag, 3% Ag and 5% Ag inhibiting E. coli at all concentrations.

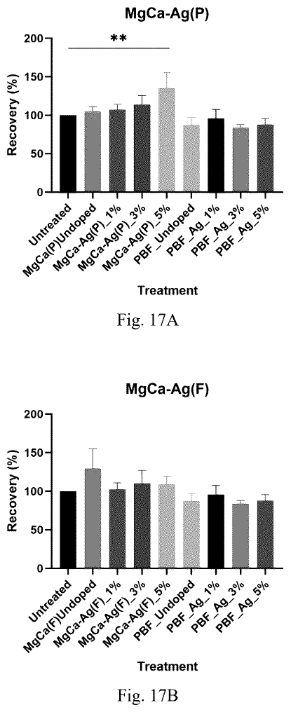

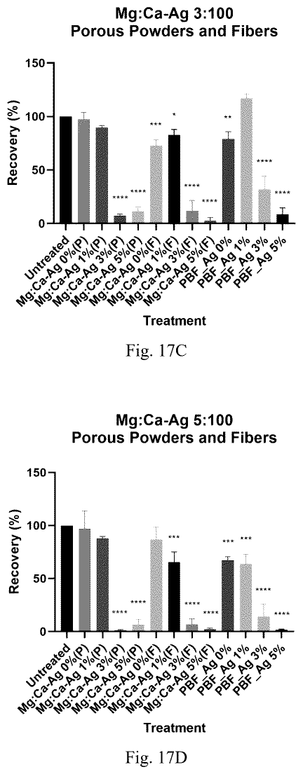

Additionally, as shown in FIGS. 17A and 17B, none of the solutions provided at a weight ratio of 1:100 glass to growth media were cytotoxic. Additionally, as shown in FIG. 17C the 1% Ag samples were also not cytotoxic when provided at a weight ratio of 1:100 glass to growth media. This indicates that solutions can be provided that have antimicrobial properties and are not cytotoxic.

### Example 9—Comparing the Antimicrobial Properties of the Porous Phosphate-Based Glass to a Non-Porous Phosphate-Based Glass

Finally, the inventors compared the antimicrobial and cytotoxicity properties of the 5% Ag porous phosphate-based glass produced in example 5 to non-porous phosphate-based glasses comprising 5% Ag. The non-porous phosphate-based glasses were produced analogously to the methods used in examples 5 and 7, except no surfactant was used to produce the glasses.

### Results

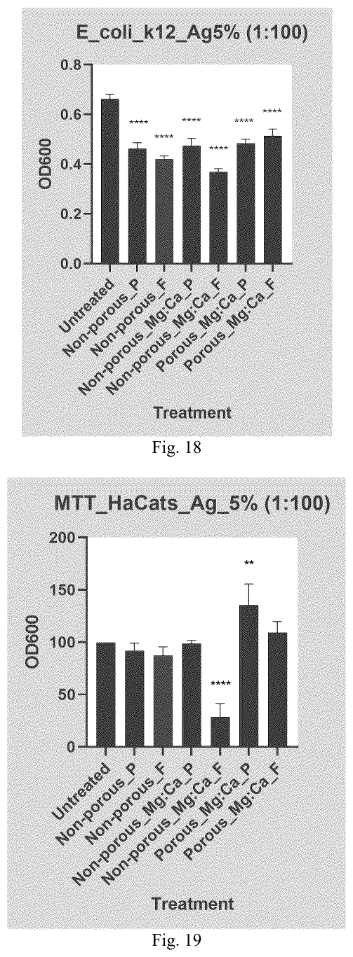

As shown in FIG. 18, all of the glasses were able to inhibit E. coli. However, as shown in FIG. 19, the porous glasses were significantly less cytotoxic than the non-porous glasses.

### Example 10—Synthesis of Porous Phosphate-Based Glass and Fibres in the System P₂O₅—CaO—Na₂O—Ga₂O₃ Using Pluronic P123 as a Surfactant

Ga³⁺ has been reported to show antibacterial properties and have other therapeutic properties. Consequently, the inventors wanted to investigate the effect doping with Ga³⁺ had on phosphate-based glass and phosphate-based glass fibres. Accordingly, the inventors prepared porous PBGs and PBGFs in the system P₂O₅—CaO—Na₂O—Ga₂O₃ using Pluronic P123 as a surfactant. The compositions prepared are summarised in Table 11.

TABLE 11
Samples of Ga-doped P₂O₅—CaO—Na₂O porous phosphate-based
glasses (powders) and phosphate-based glass fibres
synthesised using Pluronic P123

| Sample Name | Mol % Ga₂O₃ | Treatment | Morphology |
|---|---|---|---|
| COA_123 | 0 | Uncal and Cal | Powder (P) and |
| | | | fibres (F) |
| COA_0.2% Ga_123 | 0.2 | Uncal and Cal | Powder (P) and |
| | | | fibres (F) |
| COA_0.5% Ga_123 | 0.5 | Uncal and Cal | Powder (P) and |
| | | | fibres (F) |
| COA_1% Ga_123 | 1 | Uncal and Cal | Powder (P) and |
| | | | fibres (F) |
| COA_3% Ga_123 | 3 | Uncal and Cal | Powder (P) |
| COA_5% Ga_123 | 5 | Uncal and Cal | Powder (P) |
| COA_10% Ga_123 | 10 | Uncal and Cal | Powder (P) |

Coacervates were produced using a similar method to that described in Example 2 for the production of P₂O₅—CaO—Na₂O+surfactant glasses. However, in addition, gallium (III) nitrate hydrate (Ga(NO₃)₃·H₂O Alfa Aesar) was introduced to produce P₂O₅—CaO—Na₂O coacervates doped with Ga³⁺.

To produce porous P₂O₅—CaO—Na₂O doped with Ga³⁺, 100 mL of a Pluronic 123 solution (1 g Pluronic in 100 ml of water) was added to 20 mL of a 2M aqueous solution of Ca(NO₃)4H₂O. Then the mixture was added to 20 mL of 4M Na(PO₃)ₙ using a syringe pump along with different amounts of gallium nitrate hydrate aqueous 2M solution (1.2/3.6/6/12 mL for 1, 3, 5 and 10%, respectively), and stirred for one hour. The mixtures were allowed to stand for 24 hours. Each coacervate was split in half, one half to produce fibres and the other to produce powders. To produce powders, the samples were vacuum dried at room temperature for 48 h or until dry, before calcination at 300° C. (ramp speed 1° C./min to 300° C., dwell time: 30 min). To produce fibres, electrospinning was conducted using the same parameters described in Example 2, and the fibres were then calcinated as described above to yield the porous phosphate-based glass fibres. Only PBGF doped with Ga³⁺ up to 1 mol % were prepared because the 3% Ga³⁺ gel was too viscous to spin and was unsuitable for producing fibre.

### Results

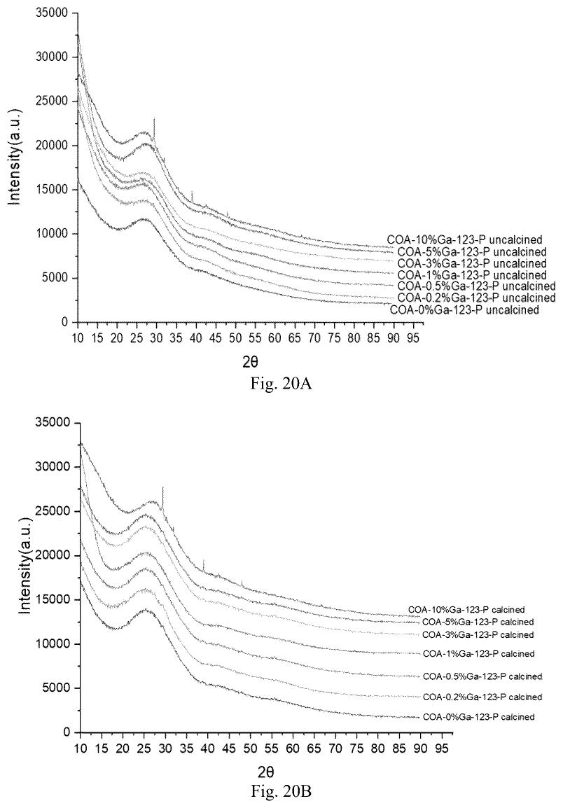

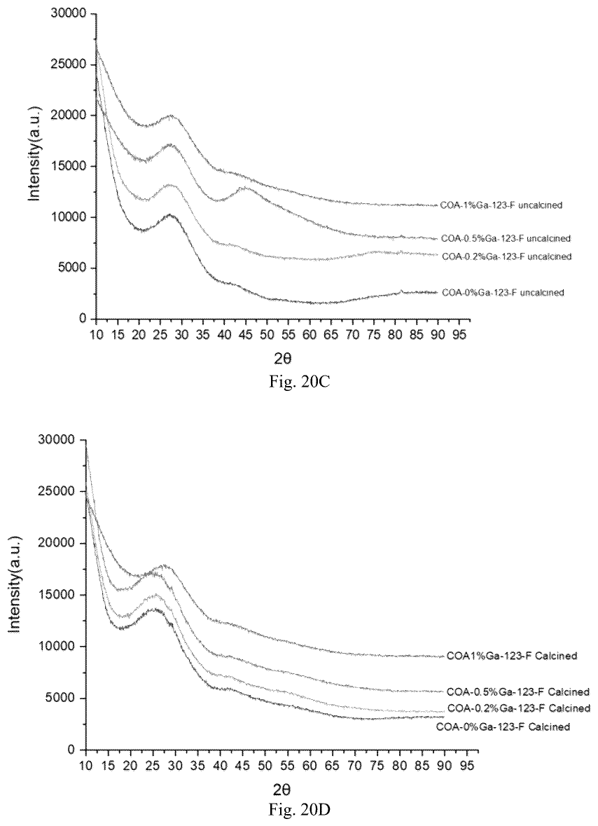

XRD characterization was used to assess the amorphous nature of the samples prepared. XRD patterns for the undoped and Ga-doped PBGs (uncalcined and calcined at 300° C.) are shown in FIGS. 20-A and 20-B, respectively. XRD patterns for the undoped and Ga-doped PBGFs (uncalcined and calcined at 300° C.) are shown in FIGS. 20-C and 20-D, respectively. A broad halo at 2θ values of between 20 and 40° confirmed the amorphous nature of the majority of the samples. Only the COA-10% Ga-123-P (uncalcined) sample shows crystalline peaks, and these peaks are assigned to NaNO₃.

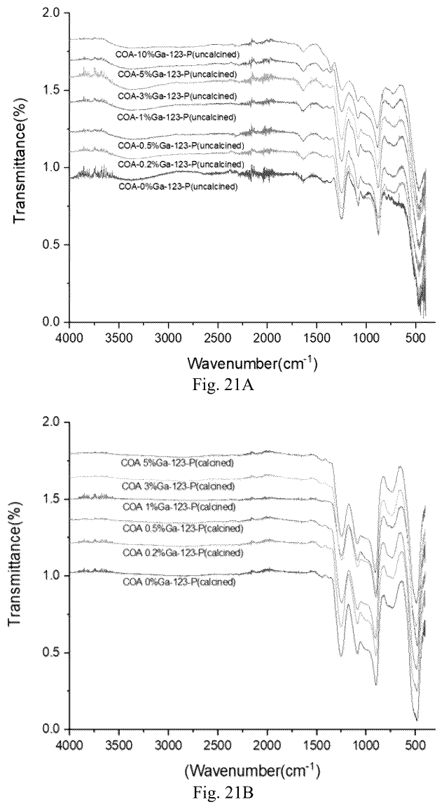

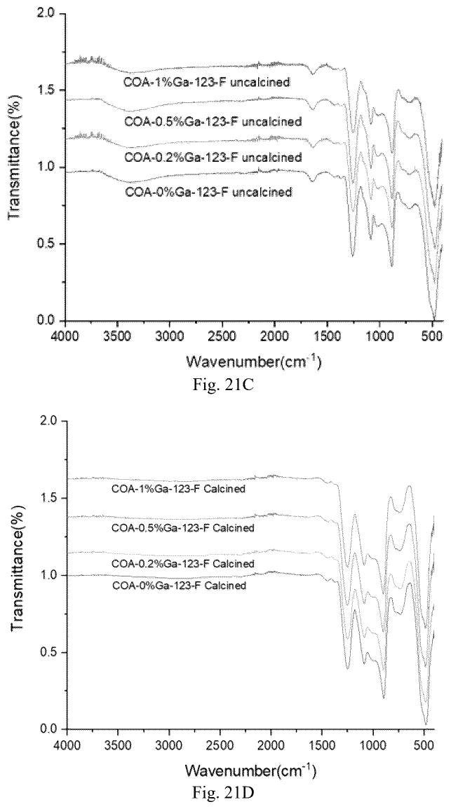

FT-IR spectra for undoped and Ga-doped PBG and PBGF (uncalcined and calcined) recorded between 4000 and 400 cm⁻¹ are shown in FIGS. 21-A to 21-D. All observed bands and corresponding wavenumbers are reported in Table 12. The bands at 1000 and 1100 cm⁻¹ are related to υs(PO₃)²⁻ and υas (PO₃)² (Q¹ phosphate units) and the peak at 1270 cm⁻¹ is related to υas (PO₂)−(Q²). In the spectra for the uncalcined PBGs and PBGFs (FIGS. 21-A and 21-C), a peak at 3300-3400 cm⁻¹ is observed υs(H—O—H) which is related to water. As expected, these bands are not observed in the spectra for the samples calcined at 300° C. (FIGS. 21-B and 21-D). The small peak at 833 cm⁻¹ in COA-10% Ga-123-P uncal could be caused by the presence of NaNO₃ (N—O symmetric stretching vibration).

TABLE 12
FT-IR Assignments for COA-Ga-123 system

| Assignment | Wavenumber (cm⁻¹) |
|---|---|
| δ(P—O—P) | 500-550 |
| υ_{S} (P—OP) | 725-750 |
| υₐₛ(P—O—P) | 900 |
| υ_{S}(PO₃)²⁻ | 1000 |
| υₐₛ(PO₃)²⁻ | 1100 |
| υₐₛ(PO₂)⁻ | 1300-1175 |
| δ(H—O—H) | 1630-1640 |
| υ(C—H) | 2900-2970 |
| υ_{S}(H—O—H) | 3300-3400 |

(υ, stretching; s, symmetric; as, asymmetric; δ, deformation)

No significant differences were observed between the spectra for the undoped samples and the samples doped with different concentrations of Ga³⁺. This indicates that the bonding in the phosphate network is not strongly affected by the addition of Ga³⁺. The only exception is the peak at 1150 cm⁻¹ which is related to the Q¹ end groups and associated with Na⁺. The intensity of this peak decreases with increasing Ga³⁺ concentration. This may be because the chain length increases as the Ga³⁺ content rises (Valappil et al., 2008). Moreover, there is no difference between the FT-IR spectra of the PBGs and the PBGFs. All bands are quite broad, confirming the amorphous nature of all samples.

The morphology of the Ga-doped PBGs and PBGFs was evaluated using SEM equipped with an EDX detector. The SEM images of COA-123-P (uncalcined) and COA-Ga-123-P (calcined) systems are shown in FIG. 22A and FIGS. 22B to 22F respectively. Before calcination, the undoped powder's surface (FIG. 22A) is not perfectly smooth. This is likely a result of the water loss under vacuum. In addition, no spherical pores were observed. However, in the calcined samples, porous features that can be ascribed to the removal of the surfactant can be observed. The SEM images show that the porosity is not homogenous. This lack of homogeneity may be a result of the surfactant (P123) not being completely removed or may result from the pores collapsing.

FIGS. 23A to 23H shows the SEM images for the undoped and Ga-doped PBGFs (uncalcined and calcined). FIGS. 23A to 23D show the SEM images for the uncalcined samples and it can be seen that the fibres are continuous and smooth and there isn't any evidence of porosity. From FIGS. 23E to 23H, it can be seen that after calcination, porous fibres are produced.

The average diameter of undoped PBGFs is in the range of 1-2 μm, whilst the average diameters for 0.2%, 0.5% and 1% Ga-doped PGFs are in the range of 1-4 μm. This shows that by increasing the amount of Ga³⁺, the diameters of fibres increases.

Tables 13 and 14 report compositions expressed in terms of oxide mol %, and elemental compositions in terms of atomic % for Ga-doped PBG and PBGF measured with EDX (energy dispersive x-ray analysis). The results show that the ratio between O and P is approximately between 2.4 and 3 in both the undoped and doped powders and fibres. Ultraphosphate is the main structure of the glasses (Brow et al., 2000).

TABLE 13
Compositions of COA-Ga-123-P measured via EDX

| Sample | Element(atomic %) | | | | | Oxide(mol %) | | | |
|---|---|---|---|---|---|---|---|---|---|
| name | O-K | Na-K | P-K | Ca-K | Ga-L | P₂O₅ | CaO | Na₂O | Ga₂O |
| 0% Ga- | 59.4 | 4.5 | 24.8 | 11.2 | — | 47.9 | 43.3 | 8.7 | — |
| 123-P | | | | | | | | | |
| CAL | | | | | | | | | |
| 0.2% Ga- | 64.6 | 4.3 | 21.8 | 9.3 | 0.1 | 48.6 | 41.5 | 9.5 | 0.2 |
| 123-P | | | | | | | | | |
| CAL | | | | | | | | | |
| 0.5% Ga- | 61.4 | 4.1 | 23.1 | 10.9 | 0.3 | 46.8 | 44.2 | 8.3 | 0.6 |
| 123-P | | | | | | | | | |
| CAL | | | | | | | | | |
| 1% Ga- | 61.4 | 5.1 | 23.1 | 9.9 | 0.4 | 46.3 | 39.6 | 10.2 | 0.8 |
| 123-P | | | | | | | | | |
| CAL | | | | | | | | | |
| 3% Ga- | 62.7 | 4.1 | 22.2 | 10.1 | 0.6 | 46.9 | 43.1 | 8.6 | 1.2 |
| 123-P | | | | | | | | | |
| CAL | | | | | | | | | |

TABLE 14
Compositions of COA-Ga-123-F measured via EDX

| Sample | Element(atomic %) | | | | | Oxide(mol %) | | | |
|---|---|---|---|---|---|---|---|---|---|
| name | O-K | Na-K | P-K | Ca-K | Ga-L | P₂O₅ | CaO | Na₂O | Ga₂O₃ |
| 0% Ga- | 60.7 | 4.1 | 25.1 | 10.1 | 0 | 50.8 | 40.8 | 8.2 | — |
| 123-F | | | | | | | | | |
| CAL | | | | | | | | | |
| 0.2% Ga- | 61.1 | 4.1 | 23.6 | 11.1 | 0.1 | 47.2 | 44.4 | 7.2 | 0.1 |
| 123-F | | | | | | | | | |
| CAL | | | | | | | | | |
| 0.5% Ga- | 58.8 | 4.6 | 25.1 | 11.2 | 0.2 | 48.3 | 42.4 | 8.8 | 0.4 |
| 123-F | | | | | | | | | |
| CAL | | | | | | | | | |
| 1% Ga | 58.8 | 4.6 | 25.4 | 10.9 | 0.3 | 48.7 | 41.8 | 8.8 | 0.6 |
| p123-F | | | | | | | | | |
| CAL | | | | | | | | | |

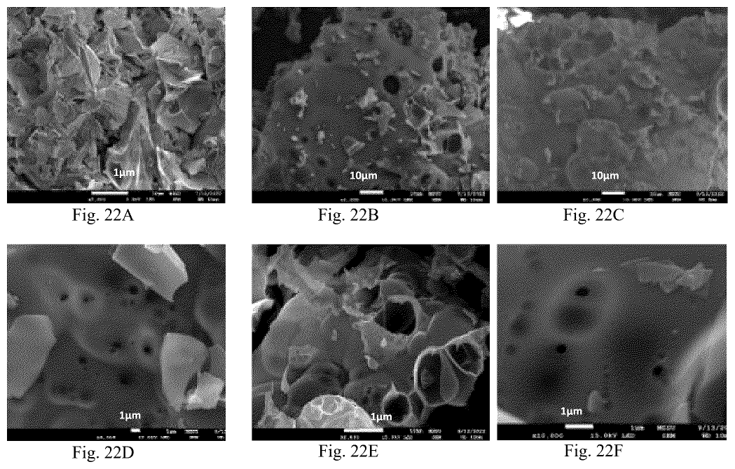

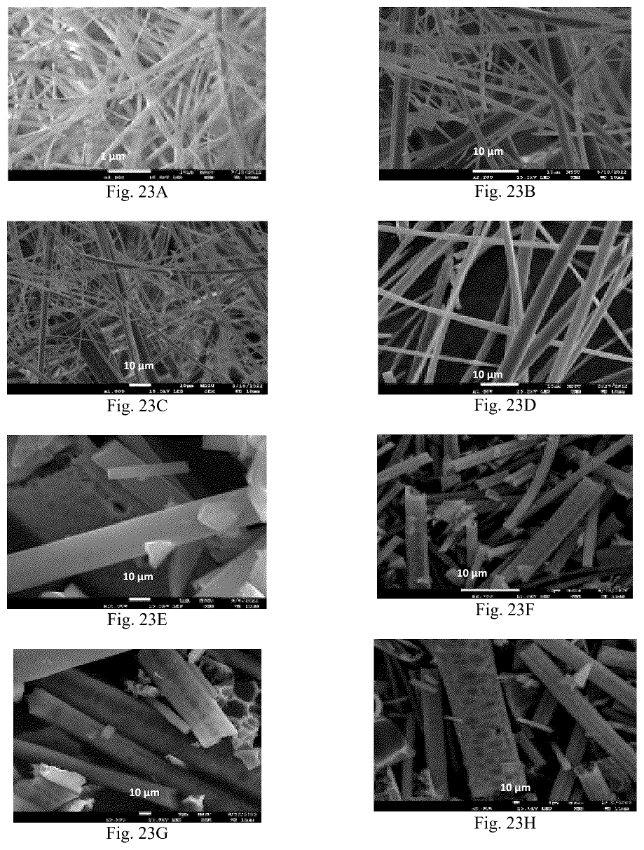

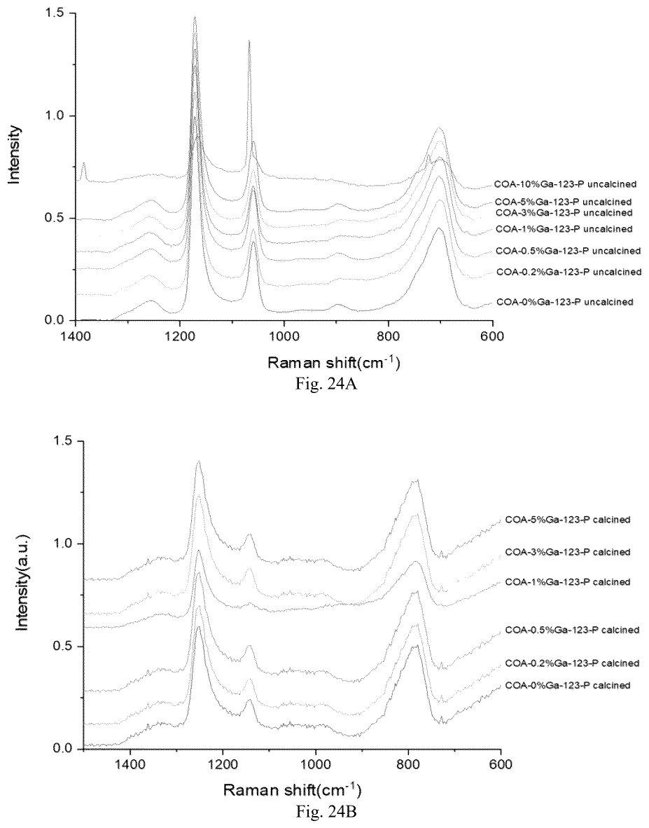

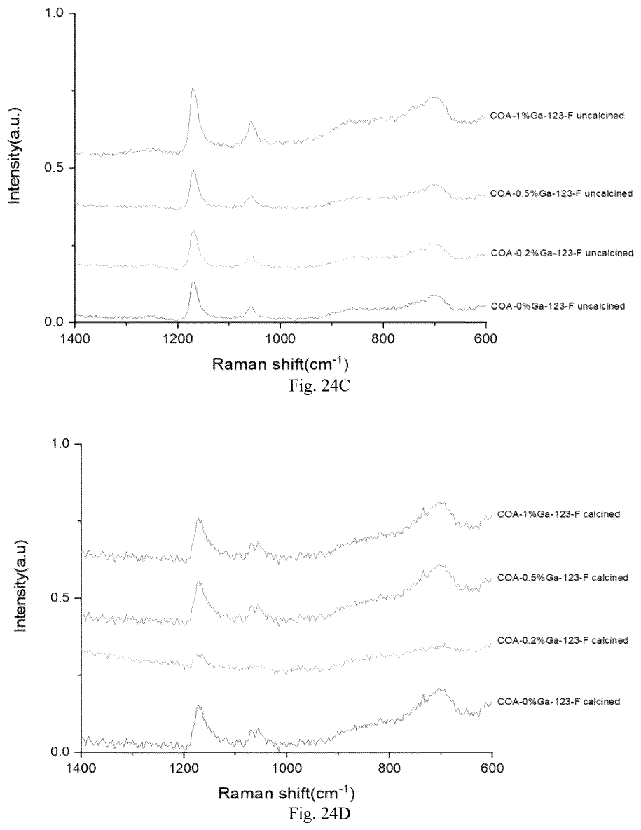

FIGS. 24A and 24B show the Raman spectra for the COA-Ga-123-P uncalcined and calcined respectively. FIGS. 24C and 24D show the Raman spectra for the COA-Ga-123-F uncalcined and calcined respectively. Table 15 shows the Raman band assignments for COA-Ga-123-P and COA-Ga-123-F. There were no significant differences between the spectra for the undoped and doped PBGs and PBGFs with different gallium concentrations. Most bands are broad, confirming that samples are mostly amorphous. The sharp peaks in spectrum for the COA-10% Ga-123-P uncalcined sample confirm the presence of crystalline material, also observed in the XRD spectrum. The Raman spectra of the calcined fibres, as shown in FIG. 24D, are quite noisy. This may be caused by the fact that the fibres are very thin, and it can be challenging to prepare a good sample for analysis.

TABLE 15
Raman bands assignments for COA-Ga-123-P and COA-Ga-123-F

| Assignment | Raman shift (cm⁻¹) |
|---|---|
| υ_{S} (P—O—P) | 695 |
| υₐₛ(P—O—P) | 890 |
| υ_{S}(PO₃)²⁻ | 1052 |
| υₛ(PO₂)⁻ | 1165 |
| υₐₛ(PO₂)⁻ | 1251 |

(υ, stretching; s, symmetric; as, asymmetric)

COA-Ga-123-F (undoped, 0.5 and 1% Ga₂O₃) were coated with 3 wt % of the antioxidant clove oil to enhance the antioxidant properties and antibacterial activity.

High encapsulation efficiency of 99.87%, 99.89% and 99.88% was observed for undoped, 0.5% and 1% Ga doped PGF, respectively. A 2,2-diphenyl-1-picrylhydrazyl (DPPH) assay was used to assess the antioxidant properties. The undoped COA-123-PGF without clove oil did not show any antioxidant property. The 1% Ga-doped PGF exhibited a higher antioxidant capacity compared to the 0.5% Ga-doped PGF. Results were confirmed by TPC assay. The highest total phenolic content (TPC) was for porous 1% Ga doped PGF coated with 3% clove oil and the lowest amount was for control sample. These results are in line with DPPH assay result.

### Example 11—Synthesis of Porous Glass Powders and Fibres in the System P₂O₅—CaO—Na₂O—Ga₂O₃ Using CTAB as a Surfactant

The inventors also prepared undoped and Ga-doped phosphate-based glasses in the system P₂O₅—CaO—Na₂O—Ga₂O₃ using CTAB as a surfactant. The synthesised samples are summarised in Table 16.

TABLE 16
Synthesised samples of Ga-doped P₂O₅—CaO—Na₂O
porous phosphate-based glasses powders and
phosphate glass fibres containing CTAB

| | Mol % | | |
|---|---|---|---|
| Sample Name | Ga₂O₃ | Treatment | Morphology |
| COA_CTAB | 0 | Uncal and Cal | Powder (P) |
| COA_0.2% Ga_CTAB | 0.2 | Uncal and Cal | Powder (P) |
| COA_0.5% Ga_CTAB | 0.5 | Uncal and Cal | Powder (P) |
| COA_1% Ga_CTAB | 1 | Uncal and Cal | Powder (P) |

To produce porous powders P₂O₅—CaO—Na₂O doped with Ga³⁺, a solution of CTAB (0.6 g CTAB in 83.4 mL water and 16.6 mL ethanol) was added to 20 mL of a 2M solution of Ca (NO₃)4H₂O. To produce porous fibres P₂O₅—CaO—Na₂O doped with Ga³⁺, a solution of CTAB (0.6 g CTAB in 95 water and 5 mL ethanol)) was added to 20 mL of a 2M solution of Ca (NO₃)4H₂O. In both cases the mixtures were then added to 20 mL of 4M Na(PO₃)ₙ using a syringe pump along with different amounts of 2M gallium nitrate hydrate aqueous solution (1.2/3.6/6/12 mL for 1, 3, 5, 10%, respectively), and stirred for one hour. The mixtures were allowed to stand for 24 hours. To produce powders, the samples were vacuum dried at room temperature for 48 h or until dry, before calcination at 350° C. (ramp speed 1° C./min to 350° C., dwell time: 30 min). 350° C. was chosen as the calcination temperature because, according to the literature, CTAB is eliminated between 250 and 300° C.

To produce fibres, electrospinning was conducted using the same parameters described in Example 2 apart from the voltage that was set between 18 and 18.5 kV, and the fibres were then calcinated as described above to yield the porous phosphate-based glass fibres.

The inventors carried out XRD, FT-IR and SEM analysis on the samples synthesised, as described for the samples in Example 10.

### Results

The XRD data was similar to that reported in Example 10 and showed that all of the COA-Ga-CTAB (uncalcined and calcined) samples were amorphous. Although the calcination temperature was raised from 300° C., for when P123 was used as the surfactant, to 350° C., no crystalline peaks were detected. Similarly, the FT-IR data showed vibrations like those reported for glasses in Example 10.

FIGS. 25A to 25D shows the SEM images for the calcined samples. It can be seen that the porosity of the PBGs is improved compared to PBGs described in Example 10, for which P123 was used as a surfactant. The porous structures prepared using CTAB appear to be spherical and smaller, with more homogeneous porosity than porous PBGs and PBGFs prepared using P123. According to the literature, using CTAB as a surfactant at low concentrations (less than 1%) produced spherical pores distribution when ethanol-water was used as a solvent (Li et al., 2006).

Tables 17 reports compositions expressed in terms of oxide mol %, and elemental compositions in terms of atomic % for Ga-doped PBG and PBGF measured with EDX (energy dispersive x-ray analysis). The results show that the ratio between O and P is approximately between 2.4 and 3 in both the undoped and doped powders and fibres. Ultraphosphate is the main structure of the glasses (Brow et al., 2000).

TABLE 17
Compositions of COA-Ga Fibres prepared
using CTAB measured via EDX

| | Element(atomic %) | | | | |
|---|---|---|---|---|---|
| Sample name | O—K | Na—K | P—K | Ca—K | Ga—L |
| COA_CTAB | 65.5 | 5.2 | 20.6 | 8.6 | 0 |
| COA_0.2% Ga_CTAB | 61.2 | 4.6 | 23.3 | 10.7 | 0.1 |
| COA_0.5% Ga_CTAB | 62.4 | 5.4 | 22.5 | 9.4 | 0.2 |
| COA_1% Ga_CTAB | 59.8 | 4.3 | 24.2 | 11.3 | 0.3 |

### Example 12—Synthesis of Porous Glass and Fibres in the System P₂O₅—CaO—Na₂O—Ce₂O₃ Using Pluronic P123 as a Surfactant

Cerium is an element in the lanthanide series which, like gallium, has been shown to have antibacterial activity. Consequently, the inventors wanted to investigate the effect of Ce³⁺ doping on phosphate-based glass (PBG) and phosphate-based glass fibre (PBGF) compositions.

Accordingly, the inventors prepared porous glass and fibres in the system P₂O₅—CaO—Na₂O—Ce₂O₃ using Pluronic P123 as a surfactant. The samples synthesised are summarised in Table 18. The coacervates were produced using a method similar to that described for the production of P₂O₅—CaO—Na₂O+surfactant glasses, as described in Example 2. However, in addition, cerium (III) nitrate hexahydrate ((Ce(NO₃)₃·6H₂O), Sigma-Aldrich, 99%) was introduced to produce P₂O₅—CaO—Na₂O coacervates doped with Ce³⁺.

TABLE 18
Synthesised samples of Ce-doped P₂O₅—CaO—Na₂O
porous phosphate-based glasses and phosphate
glass fibres containing Pluronic P123

| | Mol % | | |
|---|---|---|---|
| Sample Name | Ce₂O₃ | Treatment | Morphology |
| COA_123 | 0 | Uncal and | Powder (P) and fibres |
| | | Cal | (F) |
| COA_0.2% Ce_123 | 0.2 | Uncal and | Powder (P) and fibres |
| | | Cal | (F) |
| COA_0.5% Ce_123 | 0.5 | Uncal and | Powder (P) and fibres |
| | | Cal | (F) |
| COA_1% Ce_123 | 1 | Uncal and | Powder (P) and fibres |
| | | Cal | (F) |
| COA_3% Ce_123 | 3 | Uncal and | Powder (P) |
| | | Cal | |
| COA_5% Ce_123 | 5 | Uncal and | Powder (P) |
| | | Cal | |
| COA_10% Ce_123 | 10 | Uncal and | Powder (P) |
| | | Cal | |

To produce porous P₂O₅—CaO—Na₂O doped with Ce³⁺, a Pluronic 123 solution (1 g pluronic into 100 ml of water) was added to 20 mL of a 2M solution of Ca(NO₃)4H₂O. Then the mixture was added to 20 mL of 4M Na(PO₃)ₙ using a syringe pump along with different amounts of cerium nitrate hexahydrate 2M solution (0.08 ml, 0.2 ml, 0.4 mL, 1.2 mL, 2.0 mL and 4.0 mL for production of 0.2, 0.5, 1, 3, 5 and 10%, respectively), and stirred for one hour. The mixtures were allowed to stand for 24 hours. Each coacervate was split in half, one half to produce fibres and the other to produce powders. To produce powders, the samples were vacuum dried at room temperature for 48 h or until dry, before calcination at 300° C. (ramp speed 1° C./min to 300° C., dwell time: 30 min) to produce porous powders. To produce fibres, electrospinning was conducted using the same parameters described in Example 2, and the fibres were then calcinated as described above to yield the doped porous PG.

The inventors carried out XRD, FT-IR, Raman spectroscopy, elemental composition analysis and SEM analysis on the Ce-doped PBG and PBGF samples synthesised as described for the samples in Example 10.

### Results

The XRD results were similar to those reported in Example 10 and showed that most of the COA-Ce-123-P and COA-Ce-123-F (uncalcined and calcined) samples were amorphous. However, the spectra for the 5% and 10% Ce-doped PBGs did show some crystalline peaks related to NaNO₃. Similarly, the FT-IR data showed vibrations like those reported for glasses in Example 10. There were no differences between FT-IR spectra between COA-Ce-123-P and COA-Ce-123-F. The Raman spectra for all the PBGs and PBGFs showed relatively broad as expected for amorphous materials.

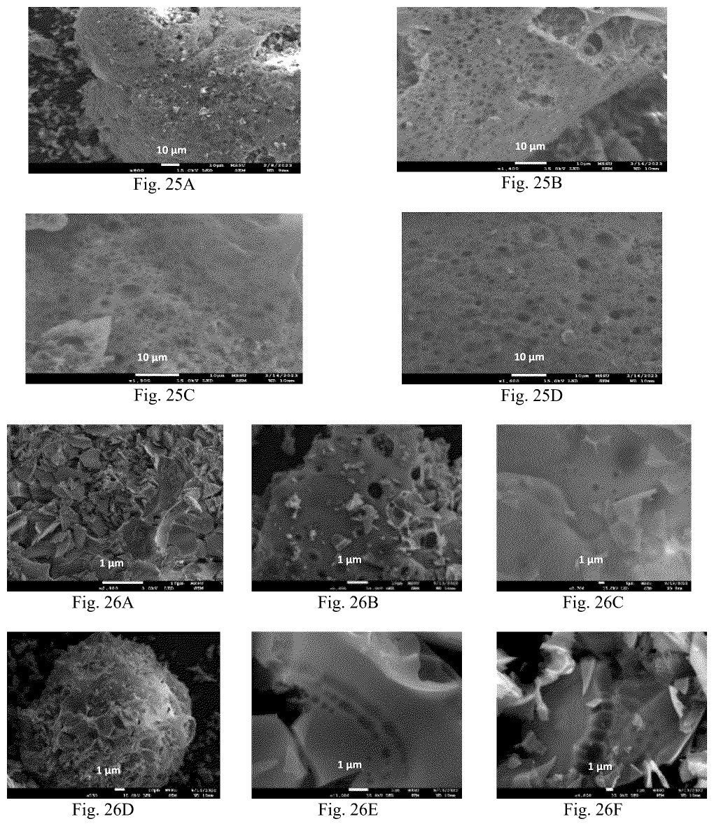

FIGS. 26A to 26F shows the SEM images for the undoped COA-123-P uncalcined (FIG. 26A) and the doped COA-Ce-123-P calcined samples (FIGS. 26B to 26F). It can be seen that the uncalcined undoped sample (FIG. 26A) does not show any features ascribed to the removal of micelles. However, some spherical porosity could be observed in the doped calcined samples (FIGS. 26B to 26F), including some regular arrays, as visible in FIGS. 26E and 26F.

FIG. 27 shows SEM images for the undoped and Ce-doped PBGF uncalcined (FIGS. 27A to 27D). FIG. 27 also shows SEM images for the undoped and Ce-doped PBGF after calcination (FIGS. 27E to 27H). Before calcination, the surface of fibres is smooth. However, after calcination, the fibres clearly show some porosity (FIGS. 27E to 27H). This is particularly evident in FIG. 27G. Like the samples described in Example 10, which were doped with Ga, the diameter of fibres increases with increasing the Ce content. The average diameter for 0.2%, 0.5% and 1% Ce-doped PBGF are in the range of 1-4 μm.

Tables 19 and 20 show elemental compositions (atomic %) and mole fractions of oxides (mol %) of PG-Ce-123 and PGF-Ce-123, respectively. The O/P ratio in COA-Ce-123-P and COA-Ce-123-F is approximately 2.5. According to the literature, glasses with a P₂O₅ content between 40 and 50 mol % and a CaO content between 20 and 40 mol % have high bioactivity and biocompatibility (Foroutan et al., 2020).

TABLE 19
Elemental composition of Ce-doped phosphate
based glasses (COA-Ce-123-P) measured via EDX

| Sample | Element(atomic %) | | | | | Oxide (mol %) | | | |
|---|---|---|---|---|---|---|---|---|---|
| name | O-K | Na-K | P-K | Ca-K | Ce-L | P₂O₅ | CaO | Na₂O | Ce₂O₃ |
| 0% Ce- | 59.4 | 4.5 | 24.8 | 11.2 | 0 | 47.9 | 43.3 | 8.7 | — |
| 123-P | | | | | | | | | |
| CAL | | | | | | | | | |
| 0.2% Ce- | 60.5 | 4.3 | 23.8 | 11.1 | 0.1 | 47.2 | 44.1 | 8.1 | 0.2 |
| 123-P | | | | | | | | | |
| CAL | | | | | | | | | |
| 0.5% Ce- | 60.1 | 4.3 | 24.3 | 10.9 | 0.2 | 48.1 | 43.1 | 8.4 | 0.4 |
| 123-P | | | | | | | | | |
| CAL | | | | | | | | | |
| 1% Ce- | 53.5 | 4.2 | 29.3 | 12.3 | 0.7 | 49.8 | 41.8 | 7.1 | 1.1 |
| 123- | | | | | | | | | |
| PCAL | | | | | | | | | |
| 3% Ce- | 63.2 | 4.9 | 25.5 | 12.7 | 1.6 | 45.1 | 43.6 | 8.5 | 2.7 |
| 123-P | | | | | | | | | |
| CAL | | | | | | | | | |

TABLE 20
Elemental composition of Ce-doped phosphate0based
glass fibres (COA-Ce-123-F) measured via EDX

| Sample | Element(atomic %) | | | | | Oxide (mol %) | | | |
|---|---|---|---|---|---|---|---|---|---|
| name | O-K | Na-K | P-K | Ca-K | Ce-L | P₂O₅ | CaO | Na₂O | Ce₂O₃ |
| 0% Ce- | 60.7 | 4.1 | 25.1 | 10.1 | 0 | 50.8 | 40.8 | 8.2 | — |
| 123-F | | | | | | | | | |
| CAL | | | | | | | | | |
| 0.2% Ce- | 53.4 | 4.3 | 28.0 | 14.1 | 0.2 | 47.1 | 45.5 | 6.9 | 0.3 |
| 123-F | | | | | | | | | |
| CAL | | | | | | | | | |
| 0.5% Ce- | 60.9 | 5.1 | 23.5 | 10.1 | 0.4 | 47.8 | 41.3 | 10.3 | 0.5 |
| 123-F | | | | | | | | | |
| CAL | | | | | | | | | |
| 1% Ce | 57.8 | 4.4 | 25.5 | 11.6 | 0.7 | 47.3 | 43.1 | 8.1 | 1.3 |
| p123-F | | | | | | | | | |
| CAL | | | | | | | | | |

### Example 13—Dissolution Studies

The controlled release of ions from the Ga and Ce doped PGs and PGFs synthesised using P123 (as described in Examples 10 and 12) was investigated using microwave plasma atomic emission spectroscopy.

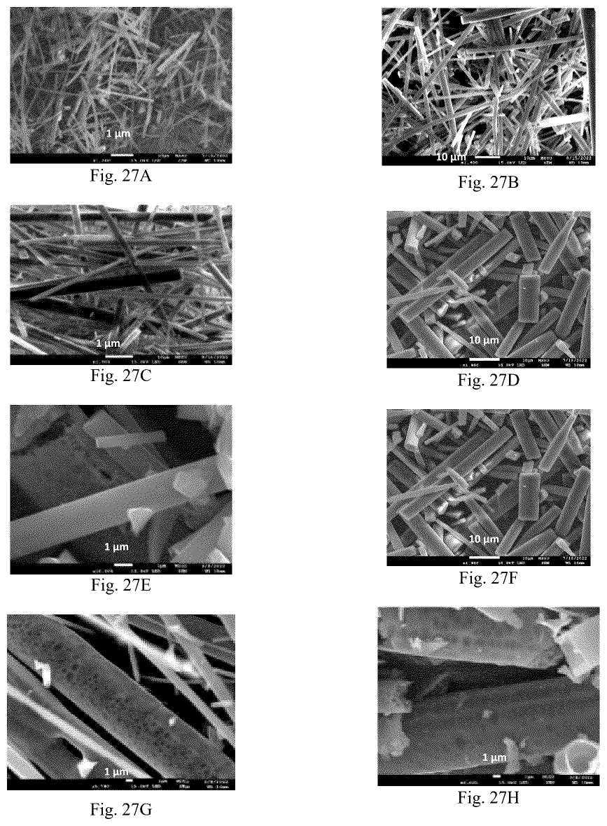

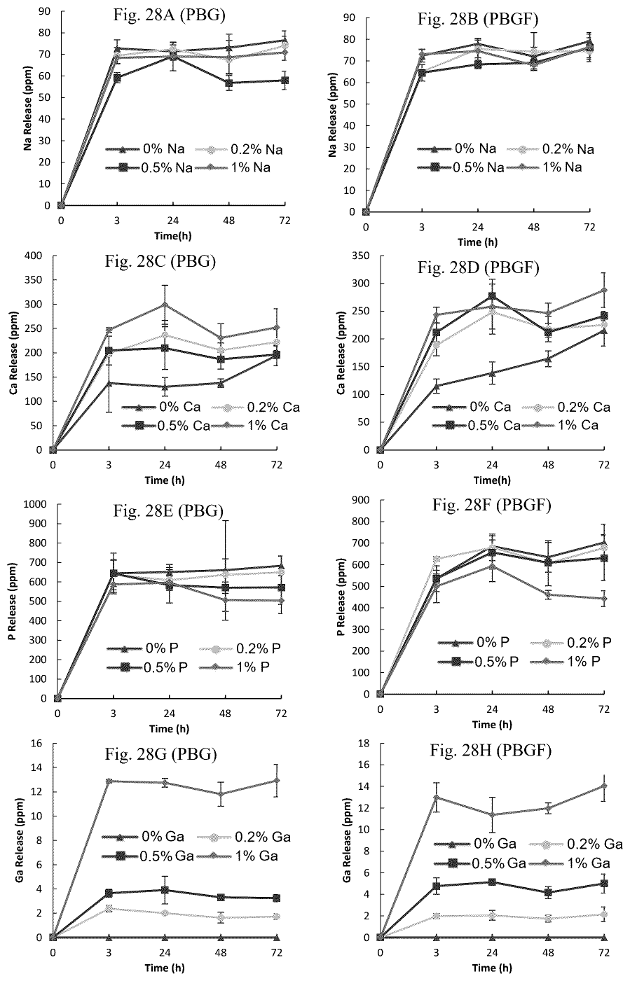

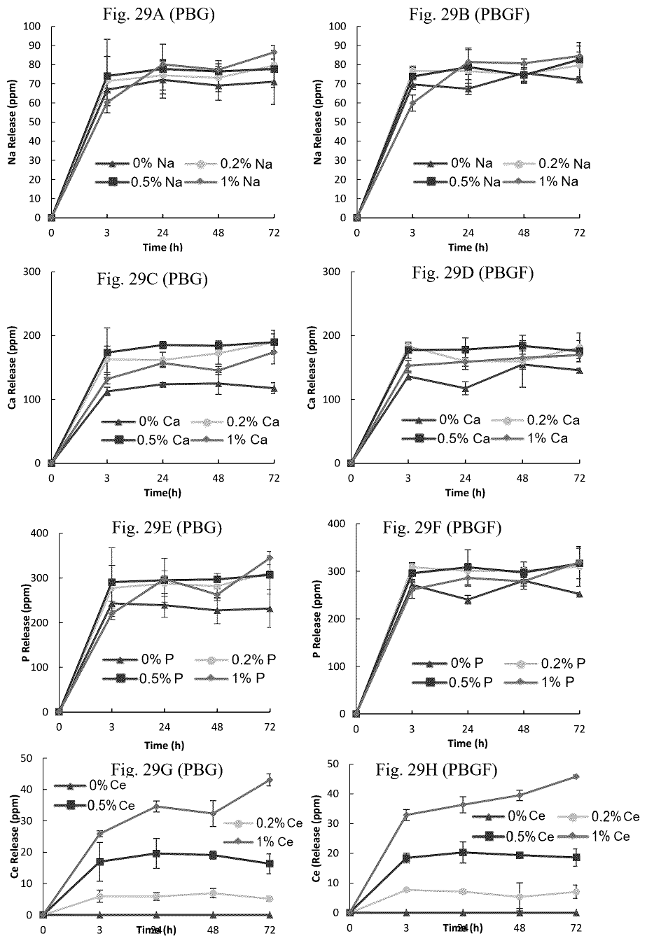

Dissolution studies were performed by immersing the PBGs and PBGFs in deionized water for up to 72 hours. The solutions obtained after 3, 24, 48 and 72 hours of immersion were then analysed to quantify the amount of phosphate anions, Ca²⁺, Na⁺ and Ga³⁺/Ce³⁺ released over time. The results for COA-Ga-123-P and COA-Ga-123-F are shown in FIGS. 28A to 28H and the results for COA-Ce-123-P and COA-Ce-123-F are shown in FIGS. 29A to 29H.

### Results

FIGS. 28A to 28H show that for Ga-doped PBGs and PBGFs, Ca²⁺ and Na⁺ are rapidly released in the first 24 h and released more slowly thereafter. It appears that with increased Ga³⁺ doping concentration, the overall release of phosphate anions is decreased. This may be caused by Ga₂O₃ causing an increase in connectivity in the glass network and the formation of strong covalent Ga—O—P bonds (Valappil et al., 2008). It is also noted that Ga₂O₃ can act as a cross-linker between two phosphates. In addition, as expected, FIGS. 28G and 28H show that the rate of and total Ga³⁺ release increases as the Ga doping concentration rises.

FIGS. 29A to 29H show that the overall release of all ions from the Ce-doped PBGs and PBGFs is greater after 72 h than the release from the Ce-free PBG and PBGF. Most of the release of phosphate anions, Ca²⁺ and Na⁺ occurs within the first 24 h. It can be seen that for all ions, the undoped samples gave a lower overall release than for the Ce-doped samples. As expected, the rate of Ce³⁺ release and total Ce³⁺ release is increased with increasing Ce-doping content.

In summary, the therapeutic ion release increases with its loading. It can be therefore controlled.

### Example 14—Antibacterial Activity and Cytocompatibility of Ga-Doped Porous Phosphate-Based Glasses and Phosphate-Based Glass Fibres

The inventors investigated the ability of the porous Ga-doped and undoped phosphate-based glass produced in Example 10 to inhibit bacteria. The inventors also assessed the cytocompatibility of the porous Ga-doped phosphate-based glasses produced in Example 10.

### Results

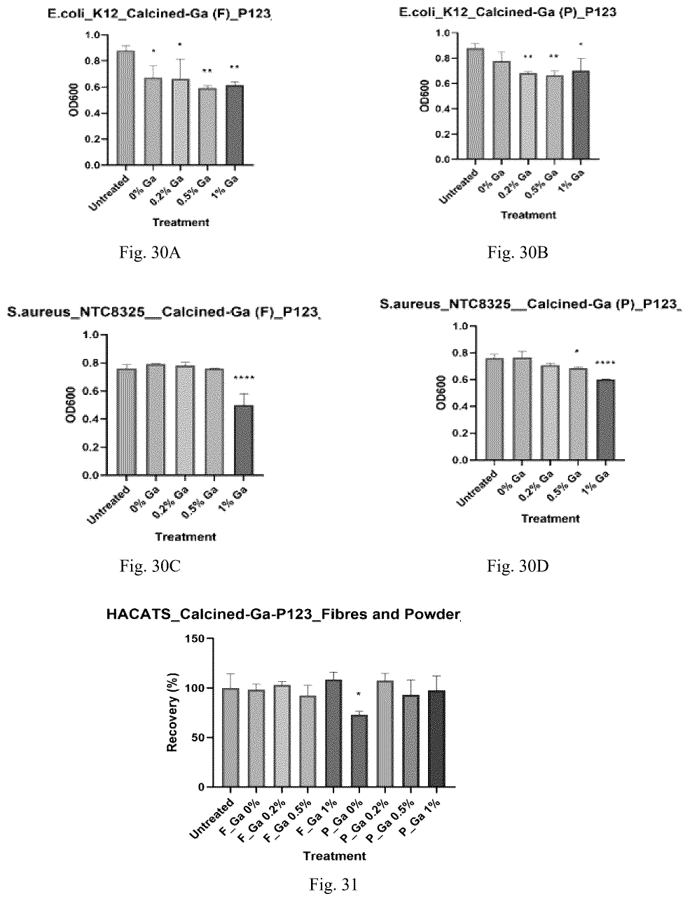

The antibacterial activity of COA-Ga-123 PBGs and PBGFs (described in Example 10) against S. aureus NCTC 8325 and E. coli K12 was studied, and the results are shown in FIGS. 30A to 30D. High OD indicates a large number of bacteria; low OD indicates few bacteria. FIGS. 30A and 30B show that all of the Ga-doped PBGF and PBG samples showed an antibacterial effect against E. coli K12. For both the PBG and PBGF, it appears that a doping concentration of 0.5% Ga has the greatest effect against E. coli K12. In addition, FIGS. 30C and 30D show that the 1% Ga doped PBGFs and PBGs showed antibacterial activity against S. aureus NCTC 8325. These results are promising as they show that Ga-doped PBGs and PBGFs show antibacterial activities against both gram-positive and gram-negative bacteria.

An MTT assay was used to determine the viability and proliferation of HaCaTs in contact with undoped and Ga-doped PBG and PBGF dissolution products for 24 hours, and the results are shown in FIG. 31. According to the MTT test, over 24 hours, the majority of cells exposed to synthesized samples did not exhibit a decrease in cell proliferation, and the majority exhibited equal or increased cell proliferation compared to control cells. Promisingly, this shows that the Ga-doped PBGs and PBGFs are not cytotoxic.

### Example 15—Assessing the Ability of Ga-Doped Porous Phosphate-Based Glass to Enhance Wound Closure

The inventors investigated the ability of porous Ga-doped phosphate-based glass produced in Example 10 to enhance wound closure on human skin biopsies.

### Results

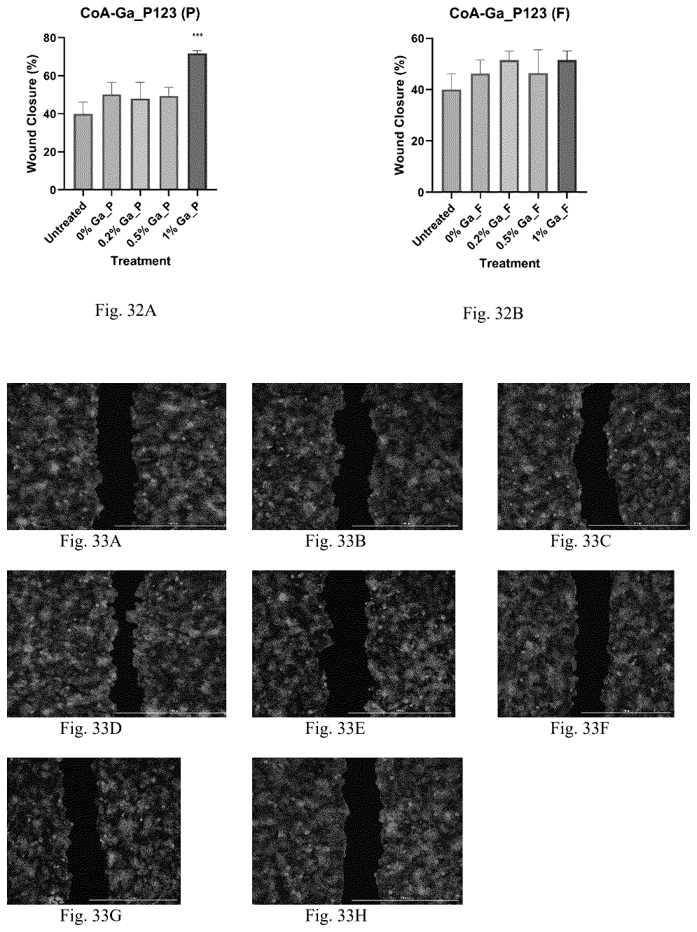

The in vitro scratch method (also called “wound closure in vitro”) was used to see how the synthesised samples would affect a damaged cell layer. As can be seen in FIGS. 32A and 32B the highest percentage of the wound closure was achieved by the porous PBG COA-1% Ga-123-P. This is affirmed by the images that are shown in FIGS. 33A to 33H. The images were taken by the camera coupled to a microscope using a 10× magnification lens.

### Methods

### Coacervation Process

The coacervation process involves a dropwise addition of a solution of polyvalent cations, e.g. calcium nitrate, to a concentrated solution of sodium polyphosphate (NaPP). Electrostatic interactions between the long polyphosphate chains and polyvalent cations lead to the formation of a coacervate phase, which can be extracted and dried to form a solid glassy material.

To produce a porous coacervate containing antibacterial silver ions (Ag⁺) and a 50% substitution of calcium ions with magnesium ions (Mg²⁺), a series of experiments were carried out using Pluronic as a surfactant. Two types of Pluronic were used, Pluronic P123 and F127, triblock copolymers of poly(ethylene oxide) poly(propylene oxide)-poly(ethylene oxide) (PEO-PPO-PEO) which have a molecular weight of 5800 g/mol (Pluronic P123, EO₂₀PO₇₀EO₂₀) and 12600 g/mol (Pluronic F127, EO₁₀₀PO₆₅EO₁₀₀) (He and Alexandridis, 2017; Pitto-Barry and Barry, 2014; Chen et al., 2009). A range of surfactant concentrations were used and the order of adding/mixing the various chemicals was changed in order to identify the best synthetic conditions to obtain the highest performance material.

To produce a coacervate containing gallium ions (Ga³⁺), a series of experiments were carried out using Pluronic 123 or CTAB (hexadecyltrimethylammonium bromide) as the surfactant. To produce a porous coacervate containing cerium ions (Ce³⁺), Pluronic 123 was used as the surfactant.

To produce undoped coacervate, a Pluronic 123 (1%) or a mixture solution of CTAB (0.6%) and ethanol (16.6%) was added to a 2M solution of Ca(NO₃)4H₂O. Then the mixture was added to a solution of 4M Na(PO₃)ₙ using a syringe pump. Doped coacervates were prepared by adding different amounts of gallium nitrate or cerium nitrate solution directly added to the mixture and stirring it for one hour.

### Characterisation

SEM images were obtained on a Thermo Fisher Apreo S SEM at an accelerating voltage of 15 kV using variable probe current. Coacervate fibres and powders were mounted onto an aluminium stub using carbon conductive tape. Energy Dispersive X-ray spectroscopy (EDX) spectra was taken on the Thermo Fisher Apreo S SEM using a Thermo Fisher Quasor II™ detector with a range of 0-20 keV with an accelerating voltage of 15 kV to determine the compositions of coacervates. X-ray diffraction (XRD) was performed using a PANalytical X'Pert spectrometer (Royston, UK) on samples in a flat plate geometry using Ni filtered Cu-Kα radiation. Data was collected using a PIXcel-1D detector with a step size of 0.0525° and a time per step of 12 s over an angular range of 2θ=10-90°. Fourier transforms infrared (FTIR) spectra were collected using a Perkin Elmer spectrometer 2000-FTIR, Seer Green, UK). Samples were scanned in the range of 1500-600 cm⁻¹. Raman spectroscopy was performed using a Renishaw In Via Reflex Raman Microscope fitted with a cooled charged coupled detector (CCD). A wavelength of 532 nm was used for all measurements.

### Ion Release Studies

10 mg of fibres/powders were placed into 10 ml of deionised water in Falcon centrifuge tubes and were placed into an incubator at 37° C. (agitated at 100 rpm) for 3 and 24 h. Each sample was performed in triplicates. Each sample was then centrifuged and filtered through a 0.45 μm Millex® GP, Millipore. Samples were stored at 4° C. until the dissolution study had concluded. All samples and standards were diluted with 2% nitric acid (Thermo Fisher 68% for trace metal analysis diluted with distilled water), to measure the concentration of ions using an Agilent MP-AES 4210. The concentration of phosphate, sodium, calcium, magnesium, silver, cerium or gallium ions were measured using standards of 0.5, 1, 2, 5, 10, 25, 50, 75, 100 ppm on an Agilent MP-AES 4210). The signals were blank corrected using 2% HNO₃ and the ratio corrected by an internal standard of beryllium.

Human Ex vivo Wound Model and Whole-Mount Staining Data

The MgCa—Ag and Ga-doped coacervate powder and fibre dissolution samples (24 h time points) were used to assess the ex vivo wound closure on human skin biopsies. The study was conducted in Hull York Medical School (HYMS) and the procedure as described by Wilkinson et al., (2021) was used. Human skin was obtained from patients undergoing reconstructive surgery at Castle Hill Hospital and Hull Royal Infirmary (Hull, UK) under full informed, written patient consent, institutional guidelines, and ethical approval (LRECs: 17/SC/0220 and 19/NE/0150).

### Cytotoxicity Study

Cell viability was assessed using HaCaT cells (AddexBio, San Diego, CA) by the MTT assay over 24 h, using 1:100, 3:100 and 5:100 of each PBF sample (24 h dissolution point) to growth media of Dulbecco's Modified Eagle Medium (DMEM) with 2 mM L-glutamine and 4% (v/v) antibiotic-antimycotic solution. Following 24 h incubation, absorbance was read on a spectrophotometer (Multiskan FC™ microplate reader) at a wavelength of 492 nm.

### Antibacterial Study

The antimicrobial effect of the dissolution products obtained were assessed against a Gram-positive, S. aureus NCTC 8325, and a Gram-negative, E. coli K12, bacterial organisms. Both strains, were cultured in Tryptic Soy Broth (TSB, Oxoid), at 37° C. with shaking at 250 rpm for 16-24 h. Then, 1 μl of the resulting overnight bacterial culture was incubated with 1, 3 and 5 μl dissolution product and 99, 97 and 95 μl of TSB in a 96 well plate, respectively which was placed into a CLARIOStar plate reader for incubation at 37° C. for 24 h. Absorbance was measured at 600 nm. The experiment was conducted in three biological replicates for each sample and the undoped glass fibre was used as a negative control.

### REFERENCES

BROW, R. K., 2000. The structure of simple phosphate glasses. Journal of Non-Crystalline Solids, 263, 1-28.
CHEN, W., PENG, J., SU, Y., ZHENG, L., WANG, L. & JIANG, Z. 2009. Separation of oil/water emulsion using Pluronic F127 modified polyethersulfone ultrafiltration membranes. Separation and Purification Technology, 66, 591-597.
DING, Y., WANG, Y. & GUO, R. 2003. Diffusion coefficients and structure properties in the pluronic F127/n-C4H9OH/H2O system. Journal of dispersion science and technology, 24, 673-681.
FOROUTAN, F., NIKOLAOU, A., KYFFIN, B. A., ELLIOTT, R. M., FELIPE-SOTELO, M., GUTIERREZ-MERINO, J. & CARTA, D. 2020. Multifunctional phosphate-based glass fibres prepared via electrospinning of coacervate precursors: controlled delivery, biocompatibility and antibacterial activity. Materialia, 14, 100939.
HE, Z. & ALEXANDRIDIS, P. 2017. Micellization thermodynamics of Pluronic P123 (EO20PO70EO20) amphiphilic block copolymer in aqueous ethylammonium nitrate (EAN) solutions. Polymers, 10, 32.
PICKUP, D. M., NEWPORT, R. J., BARNEY, E. R., KIM, J.-Y., VALAPPIL, S. P. & KNOWLES, J. C. 2014. Characterisation of phosphate coacervates for potential biomedical applications. Journal of biomaterials applications, 28, 1226-1234.
LI, T., SUN, M. & WU, S. 2022. State-of-the-Art review of electrospun gelatin-based nanofiber dressings for wound healing applications. Nanomaterials, 12 (5), 784.
PITTO-BARRY, A. & BARRY, N. P. 2014. Pluronic® block-copolymers in medicine: from chemical and biological versatility to rationalisation and clinical advances. Polymer Chemistry, 5, 3291-3297.
VALAPPIL, S. P., READY, D., ABOU NEEL, E. A., PICKUP, D. M., CHRZANOWSKI, W., O'DELL, L. A., NEWPORT, R. J., SMITH, M. E., WILSON, M. & KNOWLES, J. C 2008. Antimicrobial gallium-doped phosphate-based glasses. Advanced functional materials, 18 (5), 732-741.
WILKINSON, H. N., KIDD, A. S., ROBERTS, E. R. & HARDMAN, M. J. 2021. Human Ex vivo Wound Model and Whole-Mount Staining Approach to Accurately Evaluate Skin Repair. JoVE (Journal of Visualized Experiments), e62326.

1. A fibre comprising or consisting of a porous phosphate-based glass (PBG), wherein the phosphate-based glass has an average pore diameter of less than 1,000 nm.

2. The fibre of claim 1, wherein the fibre has an average diameter of between 0.1 and 100 μm.

3. The fibre of claim 1, wherein the phosphate-based glass comprises an alkaline earth metal oxide.

4. The fibre of claim 1, wherein the phosphate-based glass comprises calcium oxide and/or magnesium oxide, optionally wherein the phosphate-based glass comprises between 2.5 and 50 mol % calcium oxide and between 1 and 50 mol % magnesium oxide.

5. (canceled)

6. The fibre of claim 1, wherein the phosphate-based glass has an average pore diameter of between 25 and 1,000 nm.

7. The fibre of claim 1, wherein the phosphate-based glass comprises between 10 and 90 mol % phosphorus pentoxide.

8. The fibre of claim 1, wherein the phosphate-based glass comprises an alkali metal oxide, at a concentration between 1 and 50 mol %.

9. The fibre of claim 1, wherein the phosphate-based glass comprises a therapeutic agent, optionally wherein the therapeutic agent comprises a therapeutic metal or therapeutic metal cation and the phosphate-based glass comprises between 0.001 and 20 mol % of the therapeutic agent.

10. (canceled)

11. A method of producing a fibre comprising or consisting of a porous phosphate-based glass, the method comprising:
contacting a phosphate, a surfactant and an alkaline earth metal cation in a reaction solvent to provide a reaction solution; and
electrospinning at least a portion of the reaction solution to provide the fibre comprising or consisting of a porous phosphate-based glass.

12. The method of claim 11, wherein the reaction solvent comprises or consists of water and an alcohol, and the reaction solvent comprises less than 5 vol % alcohol.

13. The method of claim 11, wherein contacting the phosphate, the surfactant and the alkaline earth metal cation comprises:
providing a first solution comprising the phosphate and a first solvent;
providing a second solution comprising the surfactant, the alkaline earth metal cation and a second solvent; and
combining the first and second solutions to provide the reaction solution.

14. The method of claim 11, where the method comprises contacting the phosphate, the surfactant and the alkaline earth metal cation with a therapeutic agent in the reaction solution.

15. The method of claim 11, wherein the method comprising comprises:
allowing phase separation to occur in the reaction solution to provide a coacervate phase and a supernatant phase; and
separating the coacervate phase and the supernatant phase;
wherein the coacervate phase is the portion of reaction solution which is electrospun.

16. The method of claim 11, wherein the method comprises calcinating the fibre.

17. The method of claim 16, wherein calcinating the fibre comprises raising the temperature of the fibre to an elevated temperature at a predefined rate and holding the fibre at an elevated temperature, wherein the predefined rate is between 0.8 and 2° C./min.

18. The method of claim 11, wherein the surfactant is a copolymer, preferably a triblock copolymer, and most preferably a symmetric triblock copolymer.

19. The method of claim 18, wherein the copolymer comprises a hydrophobic block and one or more hydrophilic blocks, preferably wherein the hydrophobic block comprises poly(propylene oxide) (PPO) and the hydrophilic block comprises poly(ethylene oxide) (PEO).

20. The method of claim 11, wherein the surfactant is a quaternary ammonium salt, and preferably wherein the surfactant is cetrimonium bromide (CTAB).

21. The method of claim 11, wherein the reaction solution comprises the surfactant in an amount between 0.0001 and 30% (w/v), between 0.001 and 20% (w/v), between 0.0025 and 10% (w/v), between 0.005 and 7.5% (w/v), between 0.0075 and 5% (w/v), between 0.01 and 2.5% (w/v), between 0.025 and 1% (w/v) or between 0.05 and 0.5% (w/v).

22. (canceled)

23. (canceled)

24. A method of (a) treating an infection, (b) drug delivery, (c) bone regeneration and/or (d) wound healing, the method comprising administering the fibre of claim 1 to a patient in need thereof.
